SEQUENCE LISTING (73 sequences)

SEQ ID NO: 1 - DNA, 1401 bp, Arabidopsis thaliana
atgggcgcattagatgagggatgtttgaatctggagctcgacattggaatgagaaatggctgcattgagcttgctttcgagcaccaacctgagactttagcaatccaagacgctgtcaaacttctcttgcaaggtcttcatgaagatgtcaatcgggaaggcatcaaaaagactcctttccgtgtcgccaaggcccttcgtgaagggaccagaggttataagcaaaaggtgaaggactatgtacagagtgctctgtttccagaagcagggttggatgaaggagttgggcaagcaggaggagtcggaggacttgttgttgtcagagacctcgatcattactcttactgtgaatcttgcttgcttccttttcatgtcaagtgtcacataggttatgtcccatcgggccagagagtgttaggactgagcaagttctctagagtcactgatgttttcgccaagcggctccaagaccctcagcgtttggctgatgatatttgttcagctctccaacattgggtcaaaccagctggagtcgctgttgttcttgaatgctctcacattcacttccccagtttggacttggactctctgaacttgtctagccaccgtggatttgtgaagctactggtttcctcggggtcaggagttttcgaggatgaaagctcgaatctttggggtgaatttcagagtttcttgatgttcaaaggtgtaaaaacgcaagctttgtgcagaaatggcagctctgtgaaagagtggtgcccaagcgttaaaagctcgtctaaattatcacctgaagttgacccggaaatggtttctgctgttgtttccatcctgaaatcactgggagaagatccgttgaggaaagaactcattgctacaccaactcgattcctcaaatggatgttgaacttccaaagaaccaacctcgaaatgaagctaaacagctttaaccctgccaaagtcaatggcgaggtcaaagagaaaaggctgcactgtgagctgaacatgcccttctggtcaatgtgtgaacatcatttgcttcctttctatggagttgttcatattggctacttttgtgctgaaggatccaaccccaaccctgttggaagttcactcatgaaagcgattgtacacttttatgggttcaagcttcaagtgcaagagaggatgactcgacagatcgctgaaacgctatcgcctcttgttggcggggatgtgattgttgtggcggaagctgggcatacttgtatgatctctagaggaattgagaagtttggaagcagcaccgcgacaatcgcagttttgggtcggttttcgagtgacaattccgcaagagcgatgtttctagacaagatccatacaactaatgccttgaagacagagtcaagctctccattttga

SEQ ID NO: 2 - protein, 466 aa, Arabidopsis thaliana
MGALDEGCLNLELDIGMRNGCIELAFEHQPETLAIQDAVKLLLQGLHEDVNREGIKKTPFRVAKALREGTRGYKQKVKDYVQSALFPEAGLDEGVGQAGGVGGLVVVRDLDHYSYCESCLLPFHVKCHIGYVPSGQRVLGLSKFSRVTDVFAKRLQDPQRLADDICSALQHWVKPAGVAVVLECSHIHFPSLDLDSLNLSSHRGFVKLLVSSGSGVFEDESSNLWGEFQSFLMFKGVKTQALCRNGSSVKEWCPSVKSSSKLSPEVDPEMVSAVVSILKSLGEDPLRKELIATPTRFLKWMLNFQRTNLEMKLNSFNPAKVNGEVKEKRLHCELNMPFWSMCEHHLLPFYGVVHIGYFCAEGSNPNPVGSSLMKAIVHFYGFKLQVQERMTRQIAETLSPLVGGDVIVVAEAGHTCMISRGIEKFGSSTATIAVLGRFSSDNSARAMFLDKIHTTNALKTESSSPF

SEQ ID NO: 3 - DNA, 1371 bp, Lycopersicon esculentum
atgggcgcattagatgaagggcactatcatgcagaaatagacaacgaagttagttttgagcttggctttgaaactcagcctgaaacactggttattcaggatgctgttagagtcctattgcagggtttgggtgaagatatcaatagggaaggaatcaagaaaaccccttttcgtgttgctaaggctctaagacaaggaacaagaggttacaaacaaaaagtgaatgatattgttcatggcgcattattccctgaagctggattggagggtggaagtggtcaggctggaggagttggtgggcttgtgattgttcgagatcttgatctcttttcgtattgtgagtcttgcttgcttccattccaggttaagtgtcatgtaggttatgtcccatctggaaaaagggttgtaggattaagcaagctttctcgggttgctgatatttttgcaaaacggctccaaagtccacagcgccttgctgatgaagtttgcactgctttgcagcatggaatcaagccaacaggcgttgctgtggttctacagtgtatgcatattcattttccaaattttgaatcagcatttctcgactcgacttcccaaggatgggtaaagataacagctacctcaggttctggtgtttttgaagatgggaatgctgatgtttggactgatttctggagtctactgaaattcagaggtattagcatagacaatgctcatcgtagatcctctggccaatcatggtgcccatctcaatcttgtggcatgccaggacaagcaaattcagctatgacaaatgcagtgaattcaatacttaaatccctgggtgaagatccattgagagaagagcttgtagaaaccccatctcggtttgtgaagtggttcatgaactttagaaactctaatttggagatgaaactgaatggctttgttcgaagtagaatagacactcgaagtcctcagggtggtaattttaatgatggcatctgctctgagctcaatttgtcattctggtctcagtgcgaacatcatctactcccttttcaaggcgttgtgcacattggttatcactcttcagatggagtcaacccggtcggaagacctctagtgcaatcagtagtacatttttatggctttaaactccaagtacaggaaagggtaaccagacaaatagctgagactgtttcatcattcttaggtgaagacattatcgtagttgtggaagcaaatcacacctgtatgatatctagaggaatcgagaaatttggaagcaacacagccacatttgctgtgttgggtcgattttccactgaccctgttgcaagagcaaaatttttgcagagcctcccagactctggttctgcaggaagatga

SEQ ID NO: 4 - protein, 456 aa, Lycopersicon esculentum
MGALDEGHYHAEIDNEVSFELGFETQPETLVIQDAVRVLLQGLGEDINREGIKKTPFRVAKALRQGTRGYKQKVNDIVHGALFPEAGLEGGSGQAGGVGGLVIVRDLDLFSYCESCLLPFQVKCHVGYVPSGKRVVGLSKLSRVADIFAKRLQSPQRLADEVCTALQHGIKPTGVAVVLQCMHIHFPNFESAFLDSTSQGWVKITATSGSGVFEDGNADVWTDFWSLLKFRGISIDNAHRRSSGQSWCPSQSCGMPGQANSAMTNAVNSILKSLGEDPLREELVETPSRFVKWFMNFRNSNLEMKLNGFVRSRIDTRSPQGGNFNDGICSELNLSFWSQCEHHLLPFQGVVHIGYHSSDGVNPVGRPLVQSVVHFYGFKLQVQERVTRQIAETVSSFLGEDIIVVVEANHTCMISRGIEKFGSNTATFAVLGRFSTDPVARAKFLQSLPDSGSAGR

SEQ ID NO: 5 - DNA, 1485 bp, Oryza sativa
atgacacatctcgaggcgcgcggcaccaccaccgccatgggagcgctcgaggaggcccacctcgccgccgccatctccgcgtgcgagtgcgagtgctacgaggaggaggaggaggatgacctcgtcgagggggacggcgaggcggcggccgccgacgccatggagccggcggtgcgcgcgctgctgctggggctcggcgaggacgcgcgccgggaggggctgcggaggacgcccaagcgcgtcgccaaggccttccgcgacggcacccgaggttacaagcaaaaagtaaaggacattgtgcagggggctctctttcctgaggttggtgttgacaagaggactggttctgctggaggaactggagggcaagttgttgttcgtgatattgatcttttctcatactgtgagtcatgcttacttccattcagcatacaattccatgttggctatgtgccctctggtggaagggttgttgggttaagcaagctttcgagagtagctgatgtcttcgccaagaggttgcagaatcctcaaagactggctagtgaagtttgtggtgcattgcatgctagcatacaacctgctggtgtggctgttgctctgcaatgttggcacatacctttgccagaaaacttgaaatgcaagactttgcaaggttggattagcacttcacattcatctcgctctggagtttttgagggtgagagcagctctttttggaatgacttctcagcccttcttaagcttaggggcatagacatggagagggacagccattctgcctccatagcttggtgccctttaaggtctcatgatgtcccagtctgcaatgggcactgcaagaaggctacaaccaacggtgcaatttcacccaaatcagtaccagctccctctaatatggtttctgctgttagctcaatgctcttatcccttggagaggatcccttcaggaaagaacttgtaggtactcctcagcgttacgtgcaatggctgatgaagttcagagcatgtaacctagatgtgaagctgaatggctttacactcaataatttgagtgtataccagagtccagctggagatgctgctgaccatcgagcaatccattctgagctgcatttgccattttgtgcgcagtgcgagcaccatcttctgccgttctatggagtagtgcatattggctaccttgacggcggagatggtgaagtgattgatcgatctcattttcaggccttggttcatttttatggatgcaagcttcaggttcaagagagaatgacaaggcagatagctgaagcagtttattctgtttcgcattgtggggccatagttgttgtagaagctaaccacatttgcatgatatcaaggggaatagagaaaatcaggagtagcactgcaacgattgcagttctgggtcagtttttgacggacccttctgccaaggcacgctttctgcagaacgtagtagatacaactggtttggcagta

SEQ ID NO: 6 - protein, 495 aa, Oryza sativa
MTHLEARGTTTAMGALEEAHLAAAISACECECYEEEEEDDLVEGDGEAAAADAMEPAVRALLLGLGEDARREGLRRTPKRVAKAFRDGTRGYKQKVKDIVQGALFPEVGVDKRTGSAGGTGGQVVVRDIDLFSYCESCLLPFSIQFHVGYVPSGGRVVGLSKLSRVADVFAKRLQNPQRLASEVCGALHASIQPAGVAVALQCWHIPLPENLKCKTLQGWISTSHSSRSGVFEGESSSFWNDFSALLKLRGIDMERDSHSASIAWCPLRSHDVPVCNGHCKKATTNGAISPKSVPAPSNMVSAVSSMLLSLGEDPFRKELVGTPQRYVQWLMKFRACNLDVKLNGFTLNNLSVYQSPAGDAADHRAIHSELHLPFCAQCEHHLLPFYGVVHIGYLDGGDGEVIDRSHFQALVHFYGCKLQVQERMTRQIAEAVYSVSHCGAIVVVEANHICMISRGIEKIRSSTATIAVLGQFLTDPSAKARFLQNVVDTTGLAV

SEQ ID NO: 7 - DNA, 1855 bp, Triticum aestivum
ccacgcgtccgaatagccgctgctaccaggcgggcggggcggacaccctctgccgccgccatgggagcgctcgaggaggcgcacctcgccgccgtcgccgcctgcgggtgcgacgaggaggaggaggaagatctcctggtggaggtcggcggcggggaggcgccgggggacgccatggagccggcggtgcgcgcgctgctggcggggctcggcgaggacgaccgccgggaggggctgcgccggacgcccaagcgcgtcgccaaggccttccgcgacggcacccgaggttacagacagaaagtaaaagacattgtgcagggtgctctgtttcctgaggttggtgttgacaaaaggactggttctgctggaggaactggagggcaagttgttgttcgtgatatcgatctttattcatactgtgaatcttgcttacttccgttcagcatacaatgccatgttggatatgtgccctccggtggaagggtcgttgggttaagcaagctttcaagagtagctgacgtctttgccaagagatttcaaaaccctcagagattagctaatgaagtttgtggtgcattgcatgctagcatacaacctgctggtgttgctgttgctatgcagtgctggcacataccgttgccggagaacttcaaatgcaaaaattcgcgagctttgattagaacctcacattcatctcgttcgggagtttttgagggcgagaacagctccttttggaatgattttgtggctcttcttaagcttagaggcatagacatggagatggacagccgttctgcttctttaacttggtgccccttaaggcctcatgaggttccgctttgcaatgggcacgcaaagaagattacaaccaatggtgctagctcagcaaaatcggcatccattccatctaatatggtttctgctgtcagctcaatgctcctgtctcttggtgaggaccccctaaggaaagaacttctaggcagtcctcagcgttacgtgcaatggctgatgaggttcagagcatgcaatcttgatgtgaagctgaatggttttacacttaacagtgcaagtgtatatgagagaccaggcgaagatgctactgatcatcgagcaattggttctgagctgcatttgccattttgtgcccagtgcgagcaccacctcctgccgttctacggagtagtgcacattggttactttggcagtggagacggtgaagggatcaaccgctcacattttcaggctctggttcatttctacgggtgcaagcttcaggtccaggaaaggatgacaagacagatagctgaagcagtttattccgtctcacatcgtggagccatagttgttgtagaagcaaaccatatctgcatgatatcaaggggaatagagaaaatcaggagtagtacagcaacaattgcagttttgggtcagttttcaaccgattcttctgccaaggcatcctttctacagaacgtcttagatactgctaatcaggaagtatgagtggcttcagatcatatactgatagaatctcagcgaaaagatacaatgagtctaatccctggtcgtcatttcagcaaggcaggcactgcgacgactatatatatggtttgaagttgtcatgcagctatggcatggttgccattttaccagccattgcaggtgggagcgaccaggacctcgtcgcgtggaatgttgtgtctgaagaagattaaccagaagcaccagaaagttttggttctcccgtgttgtaccattttcgtttgaacgacatgtcaaagttatcctagcgttgtaggtgtggcggctgtgccgagcaatacagtataaatgttttaagtggttgtctcattct

SEQ ID NO: 8 - protein, 480 aa, Triticum aestivum
MGALEEAHLAAVAACGCDEEEEEDLLVEVGGGEAPGDAMEPAVRALLAGLGEDDRREGLRRTPKRVAKAFRDGTRGYRQKVKDIVQGALFPEVGVDKRTGSAGGTGGQVVVRDIDLYSYCESCLLPFSIQCHVGYVPSGGRVVGLSKLSRVADVFAKRFQNPQRLANEVCGALHASIQPAGVAVAMQCWHIPLPENFKCKNSRALIRTSHSSRSGVFEGENSSFWNDFVALLKLRGIDMEMDSRSASLTWCPLRPHEVPLCNGHAKKITTNGASSAKSASIPSNMVSAVSSMLLSLGEDPLRKELLGSPQRYVQWLMRFRACNLDVKLNGFTLNSASVYERPGEDATDHRAIGSELHLPFCAQCEHHLLPFYGVVHIGYFGSGDGEGINRSHFQALVHFYGCKLQVQERMTRQIAEAVYSVSHRGAIVVVEANHICMISRGIEKIRSSTATIAVLGQFSTDSSAKASFLQNVLDTANQEV

SEQ ID NO: 9 - DNA, 1479 bp, Zea mays
tctgacacggagccacatcattcttgttctattataaagaagtcatgtgaaatctggataatctattcccatgcaagtcatgtgtagtagaggttgtcatggtgttcttattcaaagagacagaactgtttatacaaggaagagtgacatatgtgatatgtgaggatgtttttttttgcataaccttttgttttaatgaagatatgtggggtcttattcatcatctctacttttgttttaattgcttcatgatctgtatttaacatatatctaaatctacaggctacaggcaaaaagtaaaagacatagtgcaaggtgctctgtttccagaggttggtgtggataaaaggactggatctgctggtggaactggcgggcaagtagttgttcgagacattgaacttttctcctattgtgagtcatgcttgcttccattcagcatacagtgccatgtcgggtatgttccctcaggtggaagagtggttgggttaagcaagctttctagagtatctgatgtctttgccaagagattgcaaaaccctcaaagactagctaatgaaatctgtggtgcactgcatgctagcattcaacctgctggtgtggctgttgctctgcagtgctggcacattcctttaccagaaaacttggaatgcaagactttggaaggttggattagaacttcacattcatctcgctctggagtctttgaaggtgaaagcagcactttctggagtgacttcttggctcttgttaagcttaggggcatagacgtggaggctaaggaccgtactgtttctattccttggtgccctttaaggtctcatgaggttccactctctaacgggctctgcaagaaaaactcaactaatggcatggtttctgctgttacctcgatgctcttatcactcggagaggaccccctcaggaaagagcttttaggcactcctcagcgttatgtgcagtggctgatgaagttcaaagcatgtaatctactagacgtgaagctgaatggttttacacttagtaatgttagcttatatgagaggacaggtggaggcacaactgatcatggagcaatcagatcggagctgcacttgccattttgtgctcaatgtgaacaccatcttctgcccttctacggagtagttcacattgggtactttggcaatggaagcggagaaggcattgatcggtcgcattttcaggcgttggttcatttctacgggtgcaagcttcaggttcaagaaaggatgacaaggcagatagctgaagcggtttattcagtttcgcacaatggggccatggtcgttgttgaggccaaccacatttgcatgatctcaagggggatagagaaaataaggagcaacacggcaacaattgcagttttgggccagtttttggccgacccttctgccaaggcgtgcttcttacagaatgttttggattctgttggttctgcggtatgaatcgtatcgcaag

SEQ ID NO: 10 - protein, 400 aa, Zea mays
HISKSTGYRQKVKDIVQGALFPEVGVDKRTGSAGGTGGQVVVRDIELFSYCESCLLPFSIQCHVGYVPSGGRVVGLSKLSRVSDVFAKRLQNPQRLANEICGALHASIQPAGVAVALQCWHIPLPENLECKTLEGWIRTSHSSRSGVFEGESSTFWSDFLALVKLRGIDVEAKDRTVSIPWCPLRSHEVPLSNGLCKKNSTNGMVSAVTSMLLSLGEDPLRKELLGTPQRYVQWLMKFKACNLLDVKLNGFTLSNVSLYERTGGGTTDHGAIRSELHLPFCAQCEHHLLPFYGVVHIGYFGNGSGEGIDRSHFQALVHFYGCKLQVQERMTRQIAEAVYSVSHNGAMVVVEANHICMISRGIEKIRSNTATIAVLGQFLADPSAKACFLQNVLDSVGSAV

SEQ ID NO: 11 - DNA, 2757 bp, Arabidopsis thaliana
  misc_feature (384)..(384): n is a, c, g, or t
atgaacatgaatttttcgttttgttcaacatcttctgagttatcatatccaagtgagaatgttctgagattttctgttgcaagtcggctgttttctcctaaatggaagaaaagtttcattagtttaccttgtcgtagtaaaactacgaggaaggttttggcgtcaagccgttatgtgccagggaaattggaagatttgtcggttgttaagaagagtttaccgagaagagaacctgtggagaagcttggttttgtgaggactttgttgattgataattatgatagttatacattcaatatatatcaggctctgagtactattaatggagtgcctcctgtcgttattcggaatgatgagtggacgtgggaagaagcttaccattanttatatgaagatgttgcttttgataatattgttatatcgcctggacctggttcgcctatgtgtccagctgatataggaatatgtcttcgtcttttgcttgaatgccgtgatatcccaattctaggcgtctgccttggccaccaggcactaggttatgtccatggagctcatgtggtgcatgccccggaaccagtccatggacggttgagtgggattgaacatgatgggaacatattgttttctgatattccatccgggagaaactctgattttaaggttgttagataccattcactgatcatagataaggaatcactaccaaaggaacttgtaccaatagcgtggacgatttatgatgacactggctctttctctgagaagaattcctgtgttcctgtgaataacactgggagcccacttgggaacggatctgtcattcctgtttcagaaaagttagaaaatcgaagtcattggccttcgtcccatgttaatgggaaacaagatagacacattctcatgggcatcatgcattcttcttttccccattatggtttacagtttcatccagaaagtattgctactacctatggtagtcagttatttaaaaatttcaaggacataactgtgaattattggagtcggtgcaaatctacatccctgcgtcgaagaaacataaatgacactgcaaacatgcaggtgcctgatgctactcaattgctgaaagaactttctagaactagatgtacaggaaatggttctagctattttgggaaccctaagtctctgttttctgccaagacaaatggtgtagacgtctttgatatggtggattcatcatatccaaaaccacatacaaaattgctgaggttgaaatggaagaagcatgaacgtcttgcgcataaagttggtggagtaagaaatatatttatggaactctttggcaagaatagaggaaatgatactttttggctggatacttcttctagtgacaaggctagaggacgattttctttcatgggcggtaaaggtggatctctctggaagcaattgacatttagtttatctgatcaaagtgaggttacatcaaaacatgcgggacatcttctgattgaagattctcagagttctactgagaaacaattcttggaagaaggctttcttgattttctccgtaaggagctttcatctatctcttatgatgagaaggacttcgaagagttgccttttgatttttgcggtggatacgtaggttgtattgggtatgatattaaagtggaatgtggaatgccaattaatcgtcacaaatccaacgctccagatgcatgtttcttctttgcggataatgttgtcgccattgatcatcaactcgatgacgtttatatattatcgctttacgaagagggaactgcagaaacctctttcctgaatgatactgaagagaagctcattagcttgatgggtttgtccacaagaaagttggaggatcaaactcttccagttatagattcatctcaatccaaaacaagttttgttcctgacaaatcccgagagcagtatatcaacgatgttcagagctgtatgaagtatatcaaagacggggagagctacgagctttgtctcactactcaaaacagaaggaaaataggaaatgctgatcctttgggactttatctccacctgagagagaggaatccagcaccatatgcagcatttctcaacttctcaaatgcaaatctgtctttatgctcttcgtcccctgaaaggtttcttaagctggacagaaatggaatgcttgaagcaaagccgattaagggtactatagctcgtggctccacgcctgaagaagatgaatttcttaaattgcaattgaaactcagtgagaagaatcaagccgagaatctgatgattgttgaccttctaaggaatgatctcggtcgtgtctgtgagcctggctcagtccatgtacctaacctcatggatgtagaatcatacacaacagtacatacaatggtgagcacgatccgtggactgaaaaaaacagatattagtccagtggaatgtgtaagagctgctttccctggcggttcaatgactggtgccccaaaactaagatctgttgagattctcgattctctagagaactgttcgagaggcctttactctggctcaatcgggtatttctcgtataatggtacgtttgatctgaatattgtgataagnncagtaataatacatgaagatgaagcttccattggagcaggaggagctattgttgcattatcaagtccagaagatgagtttgaggaaatgattcttaagactagagctcctgntaatgcagtcatggagtttngtagtgatcagaggagacaa

SEQ ID NO: 12 - protein, 919 aa, Arabidopsis thaliana
  misc_feature (128)..(128): Xaa can be any naturally occurring amino acid
MNMNFSFCSTSSELSYPSENVLRFSVASRLFSPKWKKSFISLPCRSKTTRKVLASSRYVPGKLEDLSVVKKSLPRREPVEKLGFVRTLLIDNYDSYTFNIYQALSTINGVPPVVIRNDEWTWEEAYHXLYEDVAFDNIVISPGPGSPMCPADIGICLRLLLECRDIPILGVCLGHQALGYVHGAHVVHAPEPVHGRLSGIEHDGNILFSDIPSGRNSDFKVVRYHSLIIDKESLPKELVPIAWTIYDDTGSFSEKNSCVPVNNTGSPLGNGSVIPVSEKLENRSHWPSSHVNGKQDRHILMGIMHSSFPHYGLQFHPESIATTYGSQLFKNFKDITVNYWSRCKSTSLRRRNINDTANMQVPDATQLLKELSRTRCTGNGSSYFGNPKSLFSAKTNGVDVFDMVDSSYPKPHTKLLRLKWKKHERLAHKVGGVRNIFMELFGKNRGNDTFWLDTSSSDKARGRFSFMGGKGGSLWKQLTFSLSDQSEVTSKHAGHLLIEDSQSSTEKQFLEEGFLDFLRKELSSISYDEKDFEELPFDFCGGYVGCIGYDIKVECGMPINRHKSNAPDACFFFADNVVAIDHQLDDVYILSLYEEGTAETSFLNDTEEKLISLMGLSTRKLEDQTLPVIDSSQSKTSFVPDKSREQYINDVQSCMKYIKDGESYELCLTTQNRRKIGNADPLGLYLHLRERNPAPYAAFLNFSNANLSLCSSSPERFLKLDRNGMLEAKPIKGTIARGSTPEEDEFLKLQLKLSEKNQAENLMIVDLLRNDLGRVCEPGSVHVPNLMDVESYTTVHTMVSTIRGLKKTDISPVECVRAAFPGGSMTGAPKLRSVEILDSLENCSRGLYSGSIGYFSYNGTFDLNIVIXXVIIHEDEASIGAGGAIVALSSPEDEFEEMILKTRAPXNAVMEFXSDQRRQ

SEQ ID NO: 13 - DNA, 2709 bp, Lycopersicon esculentum
atgaattctgccatgtcttcgtcatcatcatttatggttgcctcttcctgctgccaaaatcttcaaaccagaaaatattttcttttagcacctgaaccttttgagaagattggtatgatagatgcactccaaaaatataaccgtaaggaaaggaaggtatttatttcaagtcatttagtgcctggacatttggatgcatcgggaacaagaaaaaagtttttacatgagccagttccgaagttagaatttgtacgcacactactaattgacaactatgacagttacacttacaatattttccaagagctctcaatcattaatggaatgcctccggtggtgattcggaatgatgagtggacatggaaagaagtttatcattacctgtacgaagaaaggacgtttgacaatattgttatatcacctggtcctggctctccaacatgtccatcagatataggaatttgcctgaggctcttgcttgaatgcattgatatcccgatactaggcgtctgccttggtcaccaggcattgggatatgtgcatggtgctgaagttgtacatgcacctgaacctttccatggtcgtttgagtgatattgagcacaatggttgtcaattattccatgaaattccttcaggaagaagttctggatttaaggtggtacggtaccattcccttgtaatagacccgaaatcacttccgaaggaactcattcctatagcttggacctcaacagctgagacactccctttccagggtgtcaaacgatccaattcattcttaaatgcgtccaaagaaaataaagatatttttaatgggatgtcggagctatcagatgattcaaaggatgtgaaaggtggaaaagtcctcatgggtatcatgcattctagtaggcctcactatggattgcagttccacccagaaagtgttgcaacatgttacggaagacagcttttcaagaatttccggaaaatcacagaggattattggctcctgctgatgtcaacctccttcaatgaaagaagggctcattatgctgcatgcatgcaggtgcctaatttagacccgctgtcccgaagtgttgcaaaacgtgggcatctggtgaataaattgattgagaggagaactgccgaagtggatggaaccttaaatctatcacatccaggtcacagtgtaaaatttttgaagatgacatggaaaaaacttgattgctctgcgagccaagtaggtggagcggataatattttctgtgaactatttggagatcaagaggctaaaaacagtttttggctggatagctcttcaatagagaaggaaagggctagattttcctttatggggggaaagggtggctccctctggaagcaacttagttttaggttgtcaaatcgaagtgacagaatgtgtaaaggaggtggccatctatcagttgaagatgctaatggtcatgttatttctaaatttttggaagatggattttttgattatttagataaggagctcctatcattttgtttcgatgagaaggattatgaaggattaccatttgatttttatggtggttacattggttatatcgggtatgatcttaaagctgaatgtggtgtggcatctaatcgtcatagatccaaaacaccagatgcttgtttattctttacagataacgttattgtcattgatcatcagtatgatgacatatatactttgtcattacacgatggcagcacaagtactacttctcgcttggaagatctggagcagaggctgctcaacttgagagcttttacgcccagaaggctacaatcgcaggcatctcgaggattttctgtagtcgaactaaaatcagggttttctgctgagaaatcaagagagcagtatatcaaagacgttgagaattgtcaagagttcataaaagaaggagaaagttatgagttgtgtcttacaactcagatgagaatgaagttggggggaatagattctctggaactttatcgtaatctcagaataagaaatcctgcaccatatgctgcctggcttaatttttcaagggaaaacctaagcatatgttgttcatcacctgaaaggttcctacgattggacaggaatgctattctagaagcaaaacccataaaagggactatagctcgtggttccaccccaaaggaagatgaatttctgaaactgcaattagaatgcagtgaaaaggatcaggcggaaaatttgatgattgttgacttgttgaggaatgaccttgggcgtgtatgtgagactggctctgttcatgtcccacatctcatggaaattgaatcctatgcaacagttcataccatggtcagtacgattcgggggaaaaagcgatcagatgcaagtgcaattgattgtgttagagctgcattccctggtgggtcaatgacaggtgcaccaaagttgagatcaatggaacttcttgatcatcttgaaaattgttcgaggggcatatactcgggctgcattggatttttttcttataaccaagcatttgatctcaatattgtgatacggacggttgtcatacacgagggagaagcttcagtaggagcaggaggagccatcactgctctttcagatcctaatgatgagtatgaagaaatgcttcttaaaactcgagcaccgattaaggctgttcttgaacatcagagcagcatcttctcatcagatgctcagaagtga

SEQ ID NO: 14 - protein, 902 aa, Lycopersicon esculentum
MNSAMSSSSSFMVASSCCQNLQTRKYFLLAPEPFEKIGMIDALQKYNRKERKVFISSHLVPGHLDASGTRKKFLHEPVPKLEFVRTLLIDNYDSYTYNIFQELSIINGMPPVVIRNDEWTWKEVYHYLYEERTFDNIVISPGPGSPTCPSDIGICLRLLLECIDIPILGVCLGHQALGYVHGAEVVHAPEPFHGRLSDIEHNGCQLFHEIPSGRSSGFKVVRYHSLVIDPKSLPKELIPIAWTSTAETLPFQGVKRSNSFLNASKENKDIFNGMSELSDDSKDVKGGKVLMGIMHSSRPHYGLQFHPESVATCYGRQLFKNFRKITEDYWLLLMSTSFNERRAHYAACMQVPNLDPLSRSVAKRGHLVNKLIERRTAEVDGTLNLSHPGHSVKFLKMTWKKLDCSASQVGGADNIFCELFGDQEAKNSFWLDSSSIEKERARFSFMGGKGGSLWKQLSFRLSNRSDRMCKGGGHLSVEDANGHVISKFLEDGFFDYLDKELLSFCFDEKDYEGLPFDFYGGYIGYIGYDLKAECGVASNRHRSKTPDACLFFTDNVIVIDHQYDDIYTLSLHDGSTSTTSRLEDLEQRLLNLRAFTPRRLQSQASRGFSVVELKSGFSAEKSREQYIKDVENCQEFIKEGESYELCLTTQMRMKLGGIDSLELYRNLRIRNPAPYAAWLNFSRENLSICCSSPERFLRLDRNAILEAKPIKGTIARGSTPKEDEFLKLQLECSEKDQAENLMIVDLLRNDLGRVCETGSVHVPHLMEIESYATVHTMVSTIRGKKRSDASAIDCVRAAFPGGSMTGAPKLRSMELLDHLENCSRGIYSGCIGFFSYNQAFDLNIVIRTVVIHEGEASVGAGGAITALSDPNDEYEEMLLKTRAPIKAVLEHQSSIFSSDAQK

SEQ ID NO: 15 - DNA, 2688 bp, Oryza sativa
atggccgccctccgcctccccaccccgccgccgccccgcgcccccgcgccgtggctccattcttcccaccgccgtcgggttgcggcgccgcggggcgcgggcgggggcgggggcgggggcggggcggtcccgccgccgccggtgaggacgctcctgatcgacaactacgacagctacacctacaacatcttccaggagctctccgtcgtcaacggcgtgccgccggtggtggtgcgcaacgacgagtggacgtggagggacgtgtacaggtgggtgtacaaggagagggccttcgacaacatcgtcatctcgcctggcccgggatctccggcctgccctagcgacataggtataggcctgcggatactttgcgagtgtggagatatacccatcctgggtgtctgccttggccaccaggccctgggatttgtccatggtgctaaaatcgtccatgctcctgaagctatacatggccggcttagtgaacttgaacacaatggatgctacctctttaatcacatcccatcaggtataaactctggattcaaggtagtgcgctatcactcacttgtaatagaaccagactctctatctgaggatcttatatcaatagcatggactgcttctccaaaaatgctctcattccttgaaagcgataagcctgatataactagcagtaccttgtggggatcattggataacttattcgtaacaaaccagtcagagtgcagtaccactgatggaaaaatgcccagcataaacgatgcaagtgagttagatggctacagggttctcatgggcgttaggcactctaccaggcctcactatggagtgcagtttcaccccgagagtgttgctactcattatggaagacagatatttcaaaacttcaagaagataacaactgactttggattacagacaccattgcttcaggaaagaaaggttcacagtattggtaaactggaaagatcccaaatcagttctccagatctcaagaactttgttgcaaatgacttgttacattctgcaaggttgaaactttgggattctgttgggccttgtgctcttccaaagcgaagcagtggggacaaatgcttacggttgcaatggaaaaagattgataacttcctcaatcgcataggtggctctgaaaacattttttcagtgctttttggccatcatagcgctgaagatacattttggctggatagctcatcagttgaccagaatagggcacgattttcattcatgggcggcaagggcgggcccctttggaagcaaatgacatttcaccttgccagtcaacgagccaattgtggaggaaaccttactattcgagatgcttatggttgtactgtcagaaactttctcaaggatggtttcttggatttccttgacaaggagatgcaatccattcaatacattgaaaaggattatgaaggacttccatttgacttccatggtggatttgttggatacataggatatggtcttaaagttgaatgcgatgcatcatctaatagtgcaaaatcaagtacccctgatgcatgcttcttctttgctgataacctagttgtggttgatcacaacaatggggatgtgtacattttatcattacatgatgaatattcttctggtaatggagatggagattaccaaaactcaatacatagtttatggttagcaaatactgagaagaagcttctcaggatggatgccatggccccaagattatcgatcaatggaaactcgtcgatcaatgggaactcatttaccatatcatccagtgtgaataagcaaagatttgtcatcgagaaatcaaaagatgaatatatcagagatgtgcagagttgcctggattacataagagacggagaaagctatgaattgtgcttaactactcagatgaagagaagaacggattatatggatgctttgaaactctacctgaaattgcgaaaacaaaatccagccccttatgcagcttggcttaacttctcctcagaaaacctgagtatatgttgctcttctcctgaaaggtttctgcgactagaccgaaatgcaattctggaagcaaagccaatcaaaggtactatagcacgtggcagaacaccagaggaagatgaatgcctacgtttgcagttgaaatacagtgaaaaagatcaagctgagaacttgatgattgttgacctcctaagaaacgatcttggtaaggtttgtgaacctgggagtgtgcatgttcctcgcctcatggatgtggaatcatataaaactgtacataccatggtaagtaccattcgtggaaccaaaatgtcggacctaagccctgtcgattgtgtaaaggctgcctttccaggaggttcaatgaccggggccccgaaagttagatccatggagattcttgattcacttgaaactagtccgagaggaatatactcaggatcagttggattcttttcatataacaagacttttgacttgaatatcgtgatcagaacagttgtcctgcacaatggagaagcttcgattggggcaggcggggctattgtagcattgtcagatccagaagcagagtacaatgagatgctgcttaaagcaaaagctccaacaaaggtggttgaagagtgcagtcaacaaatatacaacccagatcgttcggattcaatgcagacaaccgtaagttag

SEQ ID NO: 16 - protein, 895 aa, Oryza sativa
MAALRLPTPPPPRAPAPWLHSSHRRRVAAPRGAGGGGGGGGAVPPPPVRTLLIDNYDSYTYNIFQELSVVNGVPPVVVRNDEWTWRDVYRWVYKERAFDNIVISPGPGSPACPSDIGIGLRILCECGDIPILGVCLGHQALGFVHGAKIVHAPEAIHGRLSELEHNGCYLFNHIPSGINSGFKVVRYHSLVIEPDSLSEDLISIAWTASPKMLSFLESDKPDITSSTLWGSLDNLFVTNQSECSTTDGKMPSINDASELDGYRVLMGVRHSTRPHYGVQFHPESVATHYGRQIFQNFKKITTDFGLQTPLLQERKVHSIGKLERSQISSPDLKNFVANDLLHSARLKLWDSVGPCALPKRSSGDKCLRLQWKKIDNFLNRIGGSENIFSVLFGHHSAEDTFWLDSSSVDQNRARFSFMGGKGGPLWKQMTFHLASQRANCGGNLTIRDAYGCTVRNFLKDGFLDFLDKEMQSIQYIEKDYEGLPFDFHGGFVGYIGYGLKVECDASSNSAKSSTPDACFFFADNLVVVDHNNGDVYILSLHDEYSSGNGDGDYQNSIHSLWLANTEKKLLRMDAMAPRLSINGNSSINGNSFTISSSVNKQRFVIEKSKDEYIRDVQSCLDYIRDGESYELCLTTQMKRRTDYMDALKLYLKLRKQNPAPYAAWLNFSSENLSICCSSPERFLRLDRNAILEAKPIKGTIARGRTPEEDECLRLQLKYSEKDQAENLMIVDLLRNDLGKVCEPGSVHVPRLMDVESYKTVHTMVSTIRGTKMSDLSPVDCVKAAFPGGSMTGAPKVRSMEILDSLETSPRGIYSGSVGFFSYNKTFDLNIVIRTVVLHNGEASIGAGGAIVALSDPEAEYNEMLLKAKAPTKVVEECSQQIYNPDRSDSMQTTVS

SEQ ID NO: 17 - DNA, 1122 bp, Arabidopsis thaliana
atggcaggtttgtcgctggagtttacagttaacacttggaatctcaggtctctctctcaagttccatgtcctttaagacatggctttcgattcccgcgtcggttgacacgaagaagaacgattctcatgtgttctgattcaagctctcagtcgtggaatgttcctgttttatctagctatgaggttggtgagaggctaaaactagcaagaggaggacaacagttcttggccatgtactcaagtgttgttgatggaattacaaccgatccagcagcgatggttcttccattggatgatcacatggttcaccgtggtcatggagtctttgacactgccctgatcatcaatggatacctttatgaattggatcagcaccttgaccgtatcttgcgatctgcatcaatggctaagatcccacttccattcgatcgagaaactattaaaagaattctcattcaaaccgtgagcgtttctggatgtagagatggatctctaagatactggctctctgcagggccaggggatttcctcctatctccatctcaatgtctcaaaccaactctctatgccattgttataaaaacgaacttcgccattaacccaataggtgtcaaggtagtgacctcgtccatccccataaagcctccagagtttgccacggtgaaaagtgttaactacctccctaacgtactctcacaaatggaagctgaggccaaaggagcttatgcaggtatttgggtgtgtaaagatgggtttattgcagaaggcccgaacatgaatgtggcgtttgttgttaatggtggtaaggagcttgtgatgccgcggtttgataacgttttgagcggatgtacagcaaagagaacacttacactcgctgaacagcttgtaagcaaggggattcttaaaactgtgaaagtaatggatgtgacagtcgaagatggaaagaaagcagatgagatgatgcttattggtagtggaattccgatcagacctgtgattcaatgggatgaagaatttattggtgaaggaaaagaaggtcctatagcaaaggcgcttcttgatctgttacttgaagatatgagatctggtccaccttccgttcgtgttcttgttccttactga

SEQ ID NO: 18 - protein, 373 aa, Arabidopsis thaliana
MAGLSLEFTVNTWNLRSLSQVPCPLRHGFRFPRRLTRRRTILMCSDSSSQSWNVPVLSSYEVGERLKLARGGQQFLAMYSSVVDGITTDPAAMVLPLDDHMVHRGHGVFDTALIINGYLYELDQHLDRILRSASMAKIPLPFDRETIKRILIQTVSVSGCRDGSLRYWLSAGPGDFLLSPSQCLKPTLYAIVIKTNFAINPIGVKVVTSSIPIKPPEFATVKSVNYLPNVLSQMEAEAKGAYAGIWVCKDGFIAEGPNMNVAFVVNGGKELVMPRFDNVLSGCTAKRTLTLAEQLVSKGILKTVKVMDVTVEDGKKADEMMLIGSGIPIRPVIQWDEEFIGEGKEGPIAKALLDLLLEDMRSGPPSVRVLVPY

SEQ ID NO: 19 - DNA, 1188 bp, Lycopersicon esculentum
atgccaatttttttcacacaaaaatttgtaaaattagctaaagatacaatggcttctttaccaactctcacaaaacccatctcagaaacttcattttttctaccaaaattaatcaatttggaattttcaagatcaaagatcagacctttaaccagatccaatgttttcaagaactcaaatttttcctctgatggacaatgttgtccaacctttgatgttccacttctttcttgctcagaggttattgagaggatgagaacaagtcgagaaggttacaagaccaagcagctttatttggcaatgtactcgagcgtttttggtggaatcacaaccgatacagctgccatggtgatacctatggatgatcacatggttcatagagggcacggtgtctttgatactgctgccattatggatggatacctttatgagttggaccaacaccttgatcgtttcctgggatccgcaaccatggccaaaatacaaattcctttcgatagggaaagcataagacagattctcatccgtacagtaagtgtttccaagtgcagaaaaggttctttaagatactggttttcggcaggacctggtgattttcaactatcttcatcaggctgtcatcaagcaactctttatgccattgtaattaaagatcaatcacctcctgatcacaacggcattaaagttgtaacgtcatccattccgataaaacccctacagtttgctgtcatgaaaagtgttaattatcttccgaatgcactttccaagatggaagcagaagaaaatgatgcatatgcagcaatttggttagatggcgatggctttgttgcagaaggaccgaacatgaatgtggcttttgttacaaaggaaaaggaccttctgatgccttgttttgataaaattctcagtggctgtacagctaaaagagtcctggttcttgcagaaaatctagtaaaggaaggtaaacttcgaggcattagagtagaaaatgtgtctgtagaggacgcgaaaagagcagatgagatgatgctaattggtagtgggattcttgtacgctcggtggtgcagtgggatgaagaaatcatcggtaatggtagagaaggtcctgtgacacaagctctgctaaatcttatcttggaagatatgaagtcagggcctcccacggtgcgagttcccgttccctattga

SEQ ID NO: 20 - protein, 395 aa, Lycopersicon esculentum
MPIFFTQKFVKLAKDTMASLPTLTKPISETSFFLPKLINLEFSRSKIRPLTRSNVFKNSNFSSDGQCCPTFDVPLLSCSEVIERMRTSREGYKTKQLYLAMYSSVFGGITTDTAAMVIPMDDHMVHRGHGVFDTAAIMDGYLYELDQHLDRFLGSATMAKIQIPFDRESIRQILIRTVSVSKCRKGSLRYWFSAGPGDFQLSSSGCHQATLYAIVIKDQSPPDHNGIKVVTSSIPIKPLQFAVMKSVNYLPNALSKMEAEENDAYAAIWLDGDGFVAEGPNMNVAFVTKEKDLLMPCFDKILSGCTAKRVLVLAENLVKEGKLRGIRVENVSVEDAKRADEMMLIGSGILVRSVVQWDEEIIGNGREGPVTQALLNLILEDMKSGPPTVRVPVPY

SEQ ID NO: 21 - DNA, 1170 bp, Oryza sativa
atgatggcctctctctccaccccacctgctaccgccggcgtctccccgtcgccccgcccttccctcctcgcgtacaagaaggctgctgggctcaccccatctccatggtgcgggtggaggagggcggcggtggccaccgcagccacgagctccaatcggaccgctgcaccagccgagaccattgtcactggaaatgatgtccctctcttgtcttttgctgaggttgcagaaaggcttgatgaattccatgcatctggaaccagaaatcaaaattacatggccatgtactctagtatttttggtggaattaccacaaatccttctgcaatggtgataccaatcgatgatcacatggtccacagaggacatggtgtttttgatactgcagctattatgaatgggcacctctatgagttagagcagcatcttgaccgcttcctgaagtctgcatcaatggccaaaatcacgctgccttttgatcggtcaacaattcggagcatacttattcaaactgtgagtgcatcaaaatgcacccaaggatcactcaggtactggctatctgttggaccaggagacttccagctatcttcagctggttgtgcaaactcagccctgtatgctattgtcattgaaagtccatcgttaccagtaccagcaggatgcaaggtgatcacatcatcgataccgataaaatctcaacagtttgcggtcatgaaaagtgtaaactacctgccaaatgcactcactaaagtggaaggcgaagaaaatggtggattcaccggcatatggttggatgatgagggtttcgtagcagagggttccaacatgaatgttggcttcgtgacacaaagcaaggagctcctcatgccccgtttcgacaagatccttagcgggtgcacggcaaaacgggttctgacccttgccaagcagctagtggcagatggaaggctcagcgggatcagttcaaggaatgtgagtgtccaggaagggaaggcggctgatgagatgatgctcattggtagtggtattcttgtcaaacctgttgttcagtgggatgatcagataattggctcaggcaaagaaggcccaattgctcagatgcttttcaacctcattcttgaggacatgagatccggtccaccttcagtccgcatccctgtctcctattga

SEQ ID NO: 22 - protein, 389 aa, Oryza sativa
MMASLSTPPATAGVSPSPRPSLLAYKKAAGLTPSPWCGWRRAAVATAATSSNRTAAPAETIVTGNDVPLLSFAEVAERLDEFHASGTRNQNYMAMYSSIFGGITTNPSAMVIPIDDHMVHRGHGVFDTAAIMNGHLYELEQHLDRFLKSASMAKITLPFDRSTIRSILIQTVSASKCTQGSLRYWLSVGPGDFQLSSAGCANSALYAIVIESPSLPVPAGCKVITSSIPIKSQQFAVMKSVNYLPNALTKVEGEENGGFTGIWLDDEGFVAEGSNMNVGFVTQSKELLMPRFDKILSGCTAKRVLTLAKQLVADGRLSGISSRNVSVQEGKAADEMMLIGSGILVKPVVQWDDQIIGSGKEGPIAQMLFNLILEDMRSGPPSVRIPVSY

SEQ ID NO: 23 - DNA, 981 bp, Oryza sativa
ggcgcctggagggaggagaggggagagatggtgagagaggaggaagaagaggaggggtgacaatgatatgtgggccatgtggcccccaccattttttaattcattcttttgttgaaactgacatgtgggtcccatgagaattattatttttcggatcgaattgccacgtaagcgctacgtcaatgctacgtcagatgaagaccgagtcaaattagccacgtaagcgccacgtcagccaaaaccaccatccaaaccgccgagggacctcatctgcactggttttgatagttgagggacccgttgtatctggtttttcgattgaaggacgaaaatcaaatttgttgacaagttaagggaccttaaatgaacttattccatttcaaaatattctgtgagccatatatccgtgggcttccaatcctcctcaaattaaagggcctttttaaaatagataattgccttctttcagtcacccataaaagtacaaaactactaccaacaagcaacatgcgcagttacacacattttctgcacatttccaccacgtcacaaagagctaagagttatccctaggacaatctcattagtgtagatacatccattaatcttttatcagaggcaaacgtaaagccgctctttatgacaaaaataggtgacacaaaagtgttatctgccacatacataacttcagaaattacccaacaccaagagaaaaataaaaaaaaatctttttgcaagctccaaatcttggaaacctttttcactctttgcagcattgtactcttgctctttttccaaccgatccatgtcaccctcaagcttctacttgatctacacgaagctcaccgtgcacacaaccatggccacaaaaaccctataaaaccccatccgatcgccatcatctcatcatcagttcatcaccaacaaacaaaagaggaaaaaaaacatatacacttctagtgattgtctgattgatcatca

SEQ ID NO: 24 - DNA, 1291 bp, Oryza sativa
caatcgaacagacagttgaagagatatggattttctaagattaattgattctctgtctaaagaaaaaaagtattattgaattaaatggaaaaagaaaaaggaaaaaggggatggcttctgcctttttgggctgaaggcggcgtgtggcccagcgtgctgcgtgggcacagccgagcgaacacacgacggagcagctacgacgaacgggggaccgagtggaccggacgaggatgtggcctaggacgagtgcacaaggctagtggactcggtcccccgcgcggtatcccgagtgggtccactcgtctgcaaacacgattcacatagagcgggagcacgcggggagccgtcctatgtgcacggcaagcaaatccgtgcgcctgggtggatttgagtgacacgggcccacgtgtagcctcacagctctccgtggtcagatgtgtaaaattatcataatatgtgtttttcacatagttaaataatatatataggcaaggtatatgggtcaataagcagtaaaaaggcttatgacatggtacaattacttacaccaatatgccttactgtctgatatattttacatgaacacacagttacaagtacgttcatttaaaaatacaaggtacttatcaattggagtgtatcaagttaatgaccacaaaacctaccaattttgctattttgaaggaacacttaaaaaaatcaataggcaagttatatagtcaataaactgcaagaaggcttatgacatggaaaaattacatacaccaatatgctttattgtccggtatattttacaagacaacaaagttataagtatgtcatttaaaaatacaagttacttatcaattgtcaagtaaatgaaaacaaacctacaaatttgttattttgaaggaacacctaaattatcaaatatagcttgctacgcaaaaatgacaacatgcttacaagttattatcatcttaaagttagactcatcttctcaagcataagagctttatggtgcaaaaacaaatataatgacaaggcaaagatacatattaagagtatggatagacatttctttaacaaactccatttgtattactccaaaagcaccagaagtttgtcatggctgagtcatgaaatgtatagttcaatcttgcaaagttgcctttccttttgtactgttttaacactacaagccatatattgtctgtacgtgcaacaaactatatcaccatgtatcccaagatgcttttttattgctatataaactagcttggtctgtctttgaactcacatcaattagctt

SEQ ID NO: 25 - DNA, 38 bp, Artificial Sequence
  Probe
aaaaagcaggctctaccatgggcgcattagatgaggga

SEQ ID NO: 26 - DNA, 40 bp, Artificial Sequence
  Probe
agaaagctgggtcttagttctttgaactagtgtttcgctg

SEQ ID NO: 27 - DNA, 35 bp, Artificial Sequence
  Probe
aaaaagcaggctctaaacgagttatgaacatgaat

SEQ ID NO: 28 - DNA, 37 bp, Artificial Sequence
  Probe
agaaagctgggtaaaactattgtctcctctgatcact

SEQ ID NO: 29 - DNA, 32 bp, Artificial Sequence
  Probe
aaggcgcgccacactctcgtctactccaagaa

SEQ ID NO: 30 - DNA, 32 bp, Artificial sequence
  Probe
caggcgcgccgatctggattttagtactggat

SEQ ID NO: 31 - DNA, 30 bp, Artificial Sequence
  Probe
ataaccatgggcgcattagatgagggatgt

SEQ ID NO: 32 - DNA, 33 bp, Artificial Sequence
  Probe
ataactagtaaatggagagcttgactctgtctt

SEQ ID NO: 33 - DNA, 20 bp, Artificial Sequence
  Probe
cctccggtgccatgaacaag

SEQ ID NO: 34 - DNA, 20 bp, Artificial sequence
  Probe
acagccctgaacacctcctg

SEQ ID NO: 35 - DNA, 22 bp, Artificial sequence
  Probe
nctcctccgccgacgccgcagn

SEQ ID NO: 36 - DNA, 20 bp, Artificial Sequence
  Probe
agggtgtcacgttgcaagac

SEQ ID NO: 37 - DNA, 20 bp, Artificial sequence
  Probe
cgctcgtctggctaagatcg

SEQ ID NO: 38 - DNA, 26 bp, Artificial sequence
  Probe
ntgcctgaaaccgaactgcccgctgn

SEQ ID NO: 39 - DNA, 774 bp, Homo sapiens
atggctcagcggatgacaacacagctgctgctccttctagtgtgggtggctgtagtaggggaggctcagacaaggattgcatgggccaggactgagcttctcaatgtctgcatgaacgccaagcaccacaaggaaaagccaggccccgaggacaagttgcatgagcagtgtcgaccctggaggaagaatgcctgctgttctaccaacaccagccaggaagcccataaggatgtttcctacctatatagattcaactggaaccactgtggagagatggcacctgcctgcaaacggcatttcatccaggacacctgcctctacgagtgctcccccaacttggggccctggatccagcaggtggatcagagctggcgcaaagagcgggtactgaacgtgcccctgtgcaaagaggactgtgagcaatggtgggaagattgtcgcacctcctacacctgcaagagcaactggcacaagggctggaactggacttcagggtttaacaagtgcgcagtgggagctgcctgccaacctttccatttctacttccccacacccactgttctgtgcaatgaaatctggactcactcctacaaggtcagcaactacagccgagggagtggccgctgcatccagatgtggttcgacccagcccagggcaaccccaatgaggaggtggcgaggttctatgctgcagccatgagtggggctgggccctgggcagcctggcctttcctgcttagcctggccctaatgctgctgtggctgctcagctag

SEQ ID NO: 40 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 41 - DNA, 1548 bp, Pisum sativum
atgagtatacttaagtgcctgggcgtgcgcgggaatcagctctgtgctgccagaaactatcttaaagtgctgggtttttcctcttttcacacagctccaaactcctctattgaaattcaaactcaagatgaagaagtagtgattgctttgggaagtaatgtaggtgatagactacataacttcaaggaagccttgaaattgatgaggaagtcaggcatacacatcacaagacatgcaagtctgtatgagacagcaccagcgtatgttactgaccaacctcgcttcctcaactctgcagtaagagcggatacgaaactcgggccacatgaattattggctgcactcaaacgaatcgagaaggatatgggccgtactgatggtataaggtatggtccaaggccaattgacttagacattttgttctatggtaaatttaaagtcagatctgatattctcacagtacctcacgaaagaatttgggaacgaccgtttgtcatggcccctttgatggatttgctgggaacagctattgacagtgatacagttgctagctggcattcattttcaggccattctggtggactaaatgcattatgggaaaagttaggtggagaatcccttattggagaggaaggtatgtatagggtaatgcctgttgcaaatggcttacttgattggtcgcgaagaacattggtcatggggattcttaatttgactccagatagtttcagtgatggagggaattttcagtctgtgaagtctgctgtttcgcaggcacggttaatgatatcagagggtgctgatataattgatattggtgctcagtctactcggccaatggcatcaaggatctctgccgaagaagaattaggtagattaatccctgtcctggaagctgtaatgtcaatacctgaggtagaaggaaaactcatatctgtggatactttctactctgaagttgcattagaagcagtacgtaaaggggctcatattataaatgatgtatctgccggaaagttagatgcaagtatgtttaaggtcatggcagagcttgatgttccttatgtcgcaatgcacatgaggggtgacccgagtacaatgcaggatagtgaaaacctgaaatatgataatgtttgcaaggatatatcgtcggaattatactcgcgggttagagaggcagaaatatcgggaatcccggcatggaggattattatggaccctggaattggattctcaaagaaaaccgaagacaatttagcggcactaacgggaatacctgatattagagaagagatttcaaaaagaagtttggccatctctcatgctcctatactaattggaccgtcaagaaagcgatttttaggtgaaatttgctctcgcccttctgcggttgatagagatcccgctaccattgcttctgtcaccgcaggtgtgttgtgtggcgcaaatattgttcgagtgcataatgttaaagataatctagatgcggtgaagctttgtgatgcaattctgaaacaaaagagttctcccataaaatttaaacagtga

SEQ ID NO: 42 - protein, 515 aa, Pisum sativum
MSILKCLGVRGNQLCAARNYLKVLGFSSFHTAPNSSIEIQTQDEEVVIALGSNVGDRLHNFKEALKLMRKSGIHITRHASLYETAPAYVTDQPRFLNSAVRADTKLGPHELLAALKRIEKDMGRTDGIRYGPRPIDLDILFYGKFKVRSDILTVPHERIWERPFVMAPLMDLLGTAIDSDTVASWHSFSGHSGGLNALWEKLGGESLIGEEGMYRVMPVANGLLDWSRRTLVMGILNLTPDSFSDGGNFQSVKSAVSQARLMISEGADIIDIGAQSTRPMASRISAEEELGRLIPVLEAVMSIPEVEGKLISVDTFYSEVALEAVRKGAHIINDVSAGKLDASMFKVMAELDVPYVAMHMRGDPSTMQDSENLKYDNVCKDISSELYSRVREAEISGIPAWRIIMDPGIGFSKKTEDNLAALTGIPDIREEISKRSLAISHAPILIGPSRKRFLGEICSRPSAVDRDPATIASVTAGVLCGANIVRVHNVKDNLDAVKLCDAILKQKSSPIKFKQ

SEQ ID NO: 43 - DNA, 1593 bp, Arabidopsis thaliana
atgaggacactctggaatcactttagtaccaattcctatatcaaaatttctccgagaatgagaagaatttctgcagcaaatttgatctcaaatcgaaatctttcaaccatttcttctactgaagatcccgagctcagagattttgtgggatttttagaatctctcaaaaactatgagaaatcaggtgtaccaaaaggagctggaactgattctgatgatggattcgatctgggtcgaatgaaacgtctcatgcttcgcctccgtaatcctcattacaaatacaaggttgttcatgttgctggaactaagggaaaaggatcaacttctgcttttctctctaatatcttacgagctggaggatattctgttggttgttattctagcccacatattctgagtatcaaagaacggatttcttgtaatggagaacctgtctctgcttccactcttaatgatcttttctattcagtcaaaccgattcttgaacagtctattcaagaggaaaatggttctttgagtcattttgagattctcactgggatagccttctctttatttgaaaaggaaaatgtcgacattgcggttatagaggctgggctaggaggagctcgagacgctacaaatgtcattgaaagttcaaatcttgctgcatcggtcataacaacgataggtgaggaacacatggcagcacttgggggttccttggaaagtatagcagaggctaaatctggaattattaaacacggtcgcccagtggttttaggtggaccgtttcttcctcatatcgagggcattctccgttctaaagcagcttcagtgtcgtcatcggttattttggcatctaatattggatctagttcttcaatcaaaggcatcattaacaaaaatgggatcgggctttgccagtcctgtgacatcgtgatacagaatgagaaagatgatcaaccaattgttgagcttagtgatgtaaacctacgtatgcttggacatcaccagctccaaaatgctgttactgccacatgtgtgtctctttgtcttcgcgatcaaggatgtgggagagtgacagatgaagcaattaggattggtttagagaacactcgtttacttgggagaagtcagtttctgacaccaaaaaaagcagagactttactattacccggagcaacagtgcttcttgatggagctcacaccaaagagtcagcgcgagctctcaaggagatgataaagaaggattttccagagaaaagattggtatttgtggttgccatggctagtgacaaagatcatgtgtcctttgcaaaagaacttctctcaggtctaaaaccagaagcagtgattctaacagaagctgacatcggtggaggtaagattagatcaacggagtcttcggcgttgaaagaatcatggataaaagctgctgatgaattggggtcaaggtctatggaagcttcagaaaacaaaactgtgttgggttccttaaagcttgcctacaagatactcagcgatgacacaacaagtagtgactcaggaatggttatagtcacgggttcgcttcacattgtatcttcagtcttagcttctcttcaacattaa

SEQ ID NO: 44 - protein, 530 aa, Arabidopsis thaliana
MRTLWNHFSTNSYIKISPRMRRISAANLISNRNLSTISSTEDPELRDFVGFLESLKNYEKSGVPKGAGTDSDDGFDLGRMKRLMLRLRNPHYKYKVVHVAGTKGKGSTSAFLSNILRAGGYSVGCYSSPHILSIKERISCNGEPVSASTLNDLFYSVKPILEQSIQEENGSLSHFEILTGIAFSLFEKENVDIAVIEAGLGGARDATNVIESSNLAASVITTIGEEHMAALGGSLESIAEAKSGIIKHGRPVVLGGPFLPHIEGILRSKAASVSSSVILASNIGSSSSIKGIINKNGIGLCQSCDIVIQNEKDDQPIVELSDVNLRMLGHHQLQNAVTATCVSLCLRDQGCGRVTDEAIRIGLENTRLLGRSQFLTPKKAETLLLPGATVLLDGAHTKESARALKEMIKKDFPEKRLVFVVAMASDKDHVSFAKELLSGLKPEAVILTEADIGGGKIRSTESSALKESWIKAADELGSRSMEASENKTVLGSLKLAYKILSDDTTSSDSGMVIVTGSLHIVSSVLASLQH

SEQ ID NO: 45 - DNA, 396 bp, Arabidopsis thaliana
atggagaaagacatggcaatgatgggagacaaactgatactgagaggcttgaaattttatggtttccatggagctattcctgaagagaagacgcttggccagatgtttatgcttgacatcgatgcttggatgtgtctcaaaaaggctggtctatcagacaacttagctgattctgtcagctatgtcgacatttacaacgtggcaaaggaagttgtagaagggtcatcaagaaaccttctggagagagttgcaggacttatagcttccaaaactctggaaatatcccctcggataacagctgttcgagtgaagctatggaagccaaatgttgcgcttattcaaagcactatcgattatttaggtgtcgagattttcagagatcgcgcaactgaataa

SEQ ID NO: 46 - protein, 131 aa, Arabidopsis thaliana
MEKDMAMMGDKLILRGLKFYGFHGAIPEEKTLGQMFMLDIDAWMCLKKAGLSDNLADSVSYVDIYNVAKEVVEGSSRNLLERVAGLIASKTLEISPRITAVRVKLWKPNVALIQSTIDYLGVEIFRDRATE

SEQ ID NO: 47 - DNA, 1560 bp, Arabidopsis thaliana
atggcaacaactactctcaatgacagtgtcaccactacacttgcttcagagcctcaaaggacttaccaagttgttgttgctgcaactaaagaaatgggtattggtaaagatgggaaattgccatggaatttgccaacagatctcaagttctttaaagacattactttgaccacttcagattcctctaagaaaaatgctgttgtgatgggtagaaagacttgggagtctattcccattaagtataggccgctttcgggtcggcttaacgttgttctaactcgttctggtgggtttgatatagccaacactgagaatgttgtcacttgtagtagtgtagattctgctcttgatttgttagctgcgccgccgtattgtttatctattgagagggtttttgttataggaggtggtgacatattgagggaagcattgaataggcctagttgtgatgctatccatttaactgagattgatacaagtgttgactgtgatacgtttatacctgcgattgatacttctgtttatcagccttggtcttcatcgtttccagtaactgaaaatggacttcggttttgcttcacgacttttgtccgtgtaaagagttctgctgatgaatcttctgatgaaagcaatgggtcacagtctcttcaatttgatgggaagaagtttttgtttcttcctaagatggtttttgatcagcatgaggagtttctgtatttgaatatggttgaagatattatctctaatggcaatgtgaagaatgataggaccgggactggtacattatcaaaatttggttgccagatgaaattcaatttacgcagaagttttccacttctgacaacaaagcgagttttctggagaggtgttgttgaggaacttctatggttcataagcggttcaaccaatgcaaaagtccttcaggaaaaaggtatccatatatgggatgggaatgcgtcaagagagtatctcgatggtatcggcctgacagagagagaggaaggcgaccttggacctgtatacggatttcaatggcgacactttggtgctaagtacacagatatgcatgctgattatactggtcaaggatttgatcaacttgtagatgtaattgacaaaatcaagaacaatcctgatgatcggcgtattataatgtcagcttggaatccttctgatcttaagctgatggcacttcctccatgccatatgtttgcacaattttatgtggcagaaggggaactctcatgtcaaatgtatcagcgttcagcagatatgggccttggtgtgccgtttaacattgcttcttactctcttcttacttgcatgctggctcatgtgtgtgatcttgttcctggtgattttatccatgttcttggggatgctcatgtatacaaaactcacgtgaggcctctgcaagagcaacttctgaatcttccaaaaccctttcctgtaatgaagataaacccggagaagaaacaaatcgattcttttgtggcttctgactttgacctcacaggctatgatcctcacaagaagatagaaatgaaaatggcggtttag

SEQ ID NO: 48 - protein, 519 aa, Arabidopsis thaliana
MATTTLNDSVTTTLASEPQRTYQVVVAATKEMGIGKDGKLPWNLPTDLKFFKDITLTTSDSSKKNAVVMGRKTWESIPIKYRPLSGRLNVVLTRSGGFDIANTENVVTCSSVDSALDLLAAPPYCLSIERVFVIGGGDILREALNRPSCDAIHLTEIDTSVDCDTFIPAIDTSVYQPWSSSFPVTENGLRFCFTTFVRVKSSADESSDESNGSQSLQFDGKKFLFLPKMVFDQHEEFLYLNMVEDIISNGNVKNDRTGTGTLSKFGCQMKFNLRRSFPLLTTKRVFWRGVVEELLWFISGSTNAKVLQEKGIHIWDGNASREYLDGIGLTEREEGDLGPVYGFQWRHFGAKYTDMHADYTGQGFDQLVDVIDKIKNNPDDRRIIMSAWNPSDLKLMALPPCHMFAQFYVAEGELSCQMYQRSADMGLGVPFNIASYSLLTCMLAHVCDLVPGDFIHVLGDAHVYKTHVRPLQEQLLNLPKPFPVMKINPEKKQIDSFVASDFDLTGYDPHKKIEMKMAV

SEQ ID NO: 49 - DNA, 1479 bp, Arabidopsis thaliana
atggcttctactggtgaaactatatgtaacaatggccaaagcgcagtttcagtaacaaggaggaggagttaccaagttgtaatagcggcgacaagagacatgggtcttggtatggacatgaaacttccttgggatctaccttcagagtatcagtttttccaagatgttacaactaggacatccgatcccacgaagaggaacgcgactataatgggaagaaaatcatgggaatctactcctctagagattcgtcctcttcctggtcggctcaatattgtcttgacaaagtctagctgccacaacattgccattgatgagaatgtactggtgtctagtagcatggaatcggctcttgaacttctagccactgagccctattccttgtccattgagaaggtctttgtcataggaggcggcgagttgttaaggaattatatgaatgcatccatctgtgatgctatccacctaaccgaaatcgatataagcgtgccatgtgacgcatttgcaccgagagtggatacttctctgtaccgtccgtggtactcatcatttccagttgtggaaaatggaattcggtatagtttcaacacttatgtccgaagaaaagatgccattgttggctccggtgaaaagaaaagtgttgctgagtcagatttgaaggagtactcgtttttgcctaagatggttttcgagaggcatgaggagtttggttacttgaatcttgttcaaaacattatatctagtggagacatgaatgacaatagtactctttctaaatttggctgtcagatgcggttcaatctgcgcaagactttcccgcttctcacgaccaagaaaatattctggcttggtgttgtagaggaaatactacaacttatcagtggttcaaacaatcccaaggaaaacggtagtcatatatgggacaccgatgaggcaaaagaatatcttgacagtttcggagtgaatgccaccgaagaagatggagacaacccattcttgcatggacttcactggaaacattgtgatgctaggtttgtgattcaagaattcagtcagctctctgatgttataaacaaaataaagaacaatcctcatgatcaaaggatcatgcttgccgcttgtaatccattagatttcaagttgtcggtatctccttgtcatacgttcacacagttctatgtggcaaatggtgaagtttcttgtcaaatataccaaagctcaaccgaagcgagtatcgggattcctttcagtatcgctacatactctcttttgacatgcatcatcgctcatgtttgcgatcttggagctggtgatttcatccatgtgattggacaagcctatattaacaaagctcacgtcaaggctatacaaaaacagcttcaaatctcccctaaacccttcccgattttgaaaatcaaccctgagaaaaagaagatggacaactttgaggcctctgatttggaactcatgaggatataa

SEQ ID NO: 50 - protein, 492 aa, Arabidopsis thaliana
MASTGETICNNGQSAVSVTRRRSYQVVIAATRDMGLGMDMKLPWDLPSEYQFFQDVTTRTSDPTKRNATIMGRKSWESTPLEIRPLPGRLNIVLTKSSCHNIAIDENVLVSSSMESALELLATEPYSLSIEKVFVIGGGELLRNYMNASICDAIHLTEIDISVPCDAFAPRVDTSLYRPWYSSFPVVENGIRYSFNTYVRRKDAIVGSGEKKSVAESDLKEYSFLPKMVFERHEEFGYLNLVQNIISSGDMNDNSTLSKFGCQMRFNLRKTFPLLTTKKIFWLGVVEEILQLISGSNNPKENGSHIWDTDEAKEYLDSFGVNATEEDGDNPFLHGLHWKHCDARFVIQEFSQLSDVINKIKNNPHDQRIMLAACNPLDFKLSVSPCHTFTQFYVANGEVSCQIYQSSTEASIGIPFSIATYSLLTCIIAHVCDLGAGDFIHVIGQAYINKAHVKAIQKQLQISPKPFPILKINPEKKKMDNFEASDLELMRI

SEQ ID NO: 51 - DNA, 1698 bp, Arabidopsis thaliana
atgaggtgtttgcaaaactccgcaaagactctacctttagcgtttaaatcggcattgctgcctttgtctcaaaggtggttctgtaaattttctcctaagccatcctcattgacaaacattttcaaggtttcaatttcaaccatggcaaacactctcaatggaaatgtgatcatgacttcaaagcctcagagtacttaccaagttgtggtagctgcaaccaaggaaatgggtattggtaaagacggaaaacttccatggaatttgcctaccgatctcaagttcttcaaggatttaactttgtccacttcggattctgctaagaagaacgctgttgttatgggtaggaaaacgtgggagtctattcccaaaaagtataggccactctctggtcgcctgaatgttgtcttgtctcgttctagcggtttcgatatagccaacactgaaaatgttgtaacttgcagtagcatagattctgctctagacttattagctgcacctccgtttagtctgtccattgagaaagtgtttgtaataggaggtggcgacatattgagggaagctttgaataaaccgagctgtgaagccatccacataactgagattgatacaagtatcgactgtgatacatttattcccacggttgacacttctgcatatcagccttggtgttcgtcgtttccaatatgtgaaaatggtcttcgattttccttcacgactcatgttcgtgtaaagagttcttctgccggtgaagcctctgatgagagtgatgggtcaaaggttcttcaagttgattggaagaagttttcttctgttcttcctaaaatgatttttgaccggcatgaggaatatctctacttaaatctggtcaaagagataatctcaaatggaaacttaaaagacgataggacagggactggtacattatctaaatttggttgtcagatgaaattcaatttacgcaggaattttccacttctgacgacaaagagagttttctggagaggtgttgttgaggaacttctatggttcataagcggttcaaccaatgcaaaggtacttcaagaaaagggtatccgtatatgggatgggaatgcgtcgagagcatatcttgatggtattggactgactgagagagaggaaggtgaccttggacccgtttatggatttcagtggagacactttggtgctaagtacacagatatgcatgctgattatactggccaaggatttgatcagctcttagatgtaattaacaagatcaagaacaatcctgatgatcgacggattataatgtcagcttggaatccttctgatcttaagctgatggcacttcctccatgccacatgtttgcacagttttatgttgcaaatggagaactttcatgtcaaatgtatcagcgttcagccgatatgggtctcggcgtaccgtttaacattgcctcatactcccttcttacatgcattcttgctcacgtctgcgatcttgttcctggtgattttatccatgtgattggagatgctcatgtttacaaaaaccatgtcaggcctctgcaagagcaacttgagaacccaccaaaaccttttcctgttttgaagataaatccagagaagaaagatatagattcttttgttgcggatgactttgagctcattggctatgatcctcacaagaaaatagatatgaaaatggctgtttag

SEQ ID NO: 52 - protein, 565 aa, Arabidopsis thaliana
MRCLQNSAKTLPLAFKSALLPLSQRWFCKFSPKPSSLTNIFKVSISTMANTLNGNVIMTSKPQSTYQVVVAATKEMGIGKDGKLPWNLPTDLKFFKDLTLSTSDSAKKNAVVMGRKTWESIPKKYRPLSGRLNVVLSRSSGFDIANTENVVTCSSIDSALDLLAAPPFSLSIEKVFVIGGGDILREALNKPSCEAIHITEIDTSIDCDTFIPTVDTSAYQPWCSSFPICENGLRFSFTTHVRVKSSSAGEASDESDGSKVLQVDWKKFSSVLPKMIFDRHEEYLYLNLVKEIISNGNLKDDRTGTGTLSKFGCQMKFNLRRNFPLLTTKRVFWRGVVEELLWFISGSTNAKVLQEKGIRIWDGNASRAYLDGIGLTEREEGDLGPVYGFQWRHFGAKYTDMHADYTGQGFDQLLDVINKIKNNPDDRRIIMSAWNPSDLKLMALPPCHMFAQFYVANGELSCQMYQRSADMGLGVPFNIASYSLLTCILAHVCDLVPGDFIHVIGDAHVYKNHVRPLQEQLENPPKPFPVLKINPEKKDIDSFVADDFELIGYDPHKKIDMKMAV

SEQ ID NO: 53 - DNA, 1716 bp, Arabidopsis thaliana
atgtttgcagtttcgatagtacctcgaaccacatcgtgccgtttgagctctgcctttctctgtcaactctcgattcctctcactcttcggctccaccatcactaccaacaccaccagcctcacctaccatctcctctctcttttcagattcattcgttaagaaagcagatcgacatggcagctcaaggaggtgattcatatgaggaagcgttggctgctttgtcgtctttgatcacgaaacgaagtcgtgctgataagagcaataaaggggatcgctttgagttagtctttgattatctcaagctacttgacctggaagaagacattttaaagatgaatgttattcatgtcgctggtaccaaaggcaagggatccacatgtacctttacagagtctattattcgaaactatggctttcgaactggactcttcacttcacctcacctcattgatgtccgggaaagatttcgtttggatggtgtggacataagtgaagagaaatttttgggatatttctggtggtgctataacaggctcaaggagagaactaacgaggagataccaatgcctacatatttccgcttccttgcattgctagcttttaaaatatttgctgcagaagaggtagatgctgctatattggaggttggattaggtggaaagtttgatgccaccaatgcggttcacaaacctgtggtatgtggtatttcttcactcggatatgaccacatggaaattctaggtgatacacttggaaaaattgctggtgagaaggctggaattttcaagcttggagttccagctttcacagtgccccaacctgatgaagccatgcgtgtccttgaagagaaagcttccgaaacagaagtgaatctcgaagtggtgcagccactaaccgcaaggctgttaagtggtcagaaacttgggcttgatggggaacaccaatatgtcaatgctggtctagcagtttcgcttgcctctatctggcttcagcaaattggtaaactagaagttccgagtcggactcagatgagtattctgcctgagaaattcatcaaagggttagctacagcgagtttgcaaggacgagcacaggtcgtccctgatcaatatactgaatctcggacttcaggagatctagtattttatctggatggagctcacagtccagaaagcatggaagcatgcgccaaatggttttcggttgcggttaagggagacaaccagtcagggagttcaggacatttggttaatggctctgcaggatcctctcatgataaatggtcaaatgaaacctgtgaacagatattgttgttcaattgtatgtcagttcgggacccaaatctactgcttccacatctaaagaatatgtgcgcaaaatacggtgtcaatttcaagaaggcattgtttgtaccaaacatgtcggtgtatcataaggttggtacagcagctgatttgccagagaatgatccacaggttgacttgtcatggcagttcacacttcagaaagtgtgggaaagccttgtgcagagtgaaagagatggagaaaaagatggtgaaagtgatggaaacagtgaggtgtttacttcactacccatggcaataaaatgtctaagggacactgtacatgagagtagctcagccacacgtttccaggtccttgtaactggttcgttacatcttgtgggcgatgtactgagattaatcagaaaatga

SEQ ID NO: 54 - protein, 571 aa, Arabidopsis thaliana
MFAVSIVPRTTSCRLSSAFLCQLSIPLTLRLHHHYQHHQPHLPSPLSFQIHSLRKQIDMAAQGGDSYEEALAALSSLITKRSRADKSNKGDRFELVFDYLKLLDLEEDILKMNVIHVAGTKGKGSTCTFTESIIRNYGFRTGLFTSPHLIDVRERFRLDGVDISEEKFLGYFWWCYNRLKERTNEEIPMPTYFRFLALLAFKIFAAEEVDAAILEVGLGGKFDATNAVHKPVVCGISSLGYDHMEILGDTLGKIAGEKAGIFKLGVPAFTVPQPDEAMRVLEEKASETEVNLEVVQPLTARLLSGQKLGLDGEHQYVNAGLAVSLASIWLQQIGKLEVPSRTQMSILPEKFIKGLATASLQGRAQVVPDQYTESRTSGDLVFYLDGAHSPESMEACAKWFSVAVKGDNQSGSSGHLVNGSAGSSHDKWSNETCEQILLFNCMSVRDPNLLLPHLKNMCAKYGVNFKKALFVPNMSVYHKVGTAADLPENDPQVDLSWQFTLQKVWESLVQSERDGEKDGESDGNSEVFTSLPMAIKCLRDTVHESSSATRFQVLVTGSLHLVGDVLRLIRK

SEQ ID NO: 55 - DNA, 1878 bp, Arabidopsis thaliana
atgctcgtttgtgggaaagggtttctaaagtgcagagcacctggagttccattcttttgtgacaaaagaaaatccttcttcactaaaactaagaggggctttcacagtttacctttgggcactggggtccgtgtttatttcaacaacaatcttaggtactcaagcaattcaatagaagttgtggagaaagctgccattaatatgggttcaaaggaagacaaggcagataatccagctttatcttcatatgatgatgccatggaagctctgtcgactcttatttctcgtcgaaaccgtggggatcgcacacctactaaaggaaatcgtgacaagcttgaacaagtcgtcacatatctcaagattttggacctggaagacaaaataaaagaactaaaagttattcatgtcgcaggaacaaaaggaaagggttcaacatgtgtcttcagtgaggcaattttacgtaattgtgggtttcgaaccggaatgtttacatctcctcacttgattgatgtgagggaaagattccgaatagatggcttggatatatctgaggaaaagtttctacagtacttctgggaatgttggaagttactgaaggagaaagctgtagatggtcttaccatgcctcctctttttcagtttctcacagtattggcatttaagatttttgtttgtgaaaaggttgatgttgctgtcattgaagttggacttgggggaaaacttgactcgacaaatgtgattcaaaaacctgttgtctgtggcattgcttctcttgggatggatcatatggacatattgggaaatacgctagctgatattgcctttcataaggctgggattttcaagcctcaaatccccgccttcacagtaccacaactctctgaagcaatggatgttcttcagaaaacagctaataatctcgaggttcctctagaagtagtagcgcctcttgaacctaaaaagttggacggagttacgcttggcttgtctggggatcatcagctagtaaatgcaggtcttgctgtttctctttcaagatgctggcttcaaagaaccgggaattggaagaagatatttccaaatgaaagcaaggaaactgagattcccgtggcattttgtcgtggtcttgcaacggcacgtcttcatgggagagctcaagttgttcatgatgtagtttcagatccgcaggactcatcagactcgatggaaactccatgcggtgatctgatcttttacttggatggtgctcacagtccacagagcatggaggcttgtggtcgatggttctcttctgcagtcagaggagacaaaagcttatccactgctgtcaatggttacatgagacatggggagtatggtacagacttaaacagagtctccaaacagattcttctgttcaactgcatggaagtgagagaccctcaagtcttacttccaaagctggtcaccacttgcgcttcctcagacactcatttctcaagagcgctttttgtcccgagcatgtctacttacaacaaagtcatttcgggtgcatcagcgattccttcagatacacgtagaaaggatttgacttggcaattcagactacaaagactatgggagaaatcaatccaagggactgatgcggggcttgaccatacactgaaacccgatggaataaccgccttaccacctcatgatttcctgtgcggagatgcacctcaatgtggtggacctgcagggacgccggtcacttccagtgccgtaatgccttcacttccattgactataaactggctaagagactgcgtacgccgaaacccttcactgaaactagaggttctggtcacaggatcgctacatcttgttggagatgtgctcaggttgctaaagagatga

SEQ ID NO: 56 - protein, 625 aa, Arabidopsis thaliana
MLVCGKGFLKCRAPGVPFFCDKRKSFFTKTKRGFHSLPLGTGVRVYFNNNLRYSSNSIEVVEKAAINMGSKEDKADNPALSSYDDAMEALSTLISRRNRGDRTPTKGNRDKLEQVVTYLKILDLEDKIKELKVIHVAGTKGKGSTCVFSEAILRNCGFRTGMFTSPHLIDVRERFRIDGLDISEEKFLQYFWECWKLLKEKAVDGLTMPPLFQFLTVLAFKIFVCEKVDVAVIEVGLGGKLDSTNVIQKPVVCGIASLGMDHMDILGNTLADIAFHKAGIFKPQIPAFTVPQLSEAMDVLQKTANNLEVPLEVVAPLEPKKLDGVTLGLSGDHQLVNAGLAVSLSRCWLQRTGNWKKIFPNESKETEIPVAFCRGLATARLHGRAQVVHDVVSDPQDSSDSMETPCGDLIFYLDGAHSPQSMEACGRWFSSAVRGDKSLSTAVNGYMRHGEYGTDLNRVSKQILLFNCMEVRDPQVLLPKLVTTCASSDTHFSRALFVPSMSTYNKVISGASAIPSDTRRKDLTWQFRLQRLWEKSIQGTDAGLDHTLKPDGITALPPHDFLCGDAPQCGGPAGTPVTSSAVMPSLPLTINWLRDCVRRNPSLKLEVLVTGSLHLVGDVLRLLKR

SEQ ID NO: 57 - DNA, 1479 bp, Arabidopsis thaliana
atggcaactgaagacgatggtgaattgtcagctcgttaccagaacacgttggatgcattgtcgtctttgatcacaaaacgtggccgtttagctagtaacaaccaatctcaccgattccgtttgctctttcattatctcaaggttcttgagcttgaagatgcagtttcacaaatgaaaatcattcatgtggccggaactaaaggaaagggatcaacatgtacatttgcggagtctattcttcgttgttacggtcttcgaactggtctcttcacatctcctcacttaatcgatgtccgagagagattccgtcttaacggcattgagataagccaggagaaatttgtgaactacttttggtgttgctttcataagctcaaggagaaaaccagcaatgaggttccaatgcctacttatttctgcttccttgctttattagctttcaagattttcacaacagaacaggttgatgttgttatactagaagttggcttaggtgggagattcgatgcgactaatgtgattcagaaacctgtcgtctgtggtatttcttctctagggtatgaccatatggagattcttggatacacacttgctgaaattgctgcagagaaagccggtatcttcaagagtggagttcctgcttttacagtggctcaacctgatgaagcaatgcgtgtactcaatgaaaaagcttcaaaattggaggtgaatcttcaggtggtggaaccgttggactcaagccagagactcgggcttcaaggcgaacatcaatatctaaacgctggtcttgctgttgcgttgtgctctacatttcttaaagagattggtattgaggacaagaatggtttggatcagacaaacggtttacccgaaaaattcatctctggattgtcaaatgcttatttgatgggacgagctatgatagtgcctgattcagaactccctgaagagattgtgtattaccttgatggagctcatagtcctgaaagcatggaagcttgcgctatatggttttcaaaacagatcaaacaaaaccaagaaagaaaccagaaaagatcagagcagatactcttgttcaattgtatgtctgttcgtgacccgagtttgcttcttccgcgattaaggagtaaatgcattgatcaaggagttgatttcaagagagccgtttttgtgccaaacgtatcagtgtacaaccaagtgggatcttcgacaaacgttggcacacgtgtcgagtcgatgtcgtggcagttcggtcttcagaggatttgggagagtttagctcgaggtgaagcaaaatctaattcaaaaagtgattctaaaggcaaagaagaagagaagagtttcgttttctcgtcacttcctgtggctgttgactggctccgggacaatgctcgccaaagtaaacaagttcgttttcaggtgttggtaactggttcattacatttggtgggtgatctcttgagatttatcaagaaatga

SEQ ID NO: 58 - protein, 492 aa, Arabidopsis thaliana
MATEDDGELSARYQNTLDALSSLITKRGRLASNNQSHRFRLLFHYLKVLELEDAVSQMKIIHVAGTKGKGSTCTFAESILRCYGLRTGLFTSPHLIDVRERFRLNGIEISQEKFVNYFWCCFHKLKEKTSNEVPMPTYFCFLALLAFKIFTTEQVDVVILEVGLGGRFDATNVIQKPVVCGISSLGYDHMEILGYTLAEIAAEKAGIFKSGVPAFTVAQPDEAMRVLNEKASKLEVNLQVVEPLDSSQRLGLQGEHQYLNAGLAVALCSTFLKEIGIEDKNGLDQTNGLPEKFISGLSNAYLMGRAMIVPDSELPEEIVYYLDGAHSPESMEACAIWFSKQIKQNQERNQKRSEQILLFNCMSVRDPSLLLPRLRSKCIDQGVDFKRAVFVPNVSVYNQVGSSTNVGTRVESMSWQFGLQRIWESLARGEAKSNSKSDSKGKEEEKSFVFSSLPVAVDWLRDNARQSKQVRFQVLVTGSLHLVGDLLRFIKK

SEQ ID NO: 59 - DNA, 669 bp, Escherichia coli
atgccatcactcagtaaagaagcggccctggttcatgaagcgttagttgcgcgaggactggaaacaccgctgcgcccgcccgtgcatgaaatggataacgaaacgcgcaaaagccttattgctggtcatatgaccgaaatcatgcagctgctgaatctcgacctggctgatgacagtttgatggaaacgccgcatcgcatcgctaaaatgtatgtcgatgaaattttctccggtctggattacgccaatttcccgaaaatcaccctcattgaaaacaaaatgaaggtcgatgaaatggtcaccgtgcgcgatatcactctgaccagcacctgtgaacaccattttgttaccatcgatggcaaagcgacggtggcctatatcccgaaagattcggtgatcggtctgtcaaaaattaaccgcattgtgcagttctttgcccagcgtccgcaggtgcaggaacgtctgacgcagcaaattcttattgcgctacaaacgctgctgggcaccaataacgtggctgtctcgatcgacgcggtgcattactgcgtgaaggcgcgtggcatccgcgatgcaaccagtgccacgacaacgacctctcttggtggattgttcaaatccagtcagaatacgcgccacgagtttctgcgcgctgtgcgtcatcacaactga

SEQ ID NO: 60 - protein, 222 aa, Escherichia coli
MPSLSKEAALVHEALVARGLETPLRPPVHEMDNETRKSLIAGHMTEIMQLLNLDLADDSLMETPHRIAKMYVDEIFSGLDYANFPKITLIENKMKVDEMVTVRDITLTSTCEHHFVTIDGKATVAYIPKDSVIGLSKINRIVQFFAQRPQVQERLTQQILIALQTLLGTNNVAVSIDAVHYCVKARGIRDATSATTTTSLGGLFKSSQNTRHEFLRAVRHHN

SEQ ID NO: 61 - DNA, 726 bp, Mus musculus
atggagaagccgcggggagtcaggtgcaccaatgggttctccgagcgggagctgccgcggcccggggccagcccgcctgccgagaagtcccggccgcccgaggccaagggcgcacagccggccgacgcctggaaggcagggcggcaccgcagcgaggaggaaaaccaggtgaacctccccaaactggcggctgcttactcgtccattctgctctcgctgggcgaggacccccagcggcaggggctgctcaagacgccctggagggcggccaccgccatgcagtacttcaccaagggataccaggagaccatctcagatgtcctgaatgatgctatatttgatgaagatcatgacgagatggtgattgtgaaggacatagatatgttctccatgtgtgagcatcaccttgttccatttgtaggaagggtccatattggctatcttcctaacaagcaagtccttggtctcagtaaacttgccaggattgtagaaatctacagtagacgactacaagttcaagagcgcctcaccaaacagattgcggtggccatcacagaagccttgcagcctgctggcgttggagtagtgattgaagcgacacacatgtgcatggtaatgcgaggcgtgcagaaaatgaacagcaagactgtcactagcaccatgctgggcgtgttccgggaagaccccaagactcgggaggagttcctcacactaatcaggagctga

SEQ ID NO: 62 - protein, 241 aa, Mus musculus
MEKPRGVRCTNGFSERELPRPGASPPAEKSRPPEAKGAQPADAWKAGRHRSEEENQVNLPKLAAAYSSILLSLGEDPQRQGLLKTPWRAATAMQYFTKGYQETISDVLNDAIFDEDHDEMVIVKDIDMFSMCEHHLVPFVGRVHIGYLPNKQVLGLSKLARIVEIYSRRLQVQERLTKQIAVAITEALQPAGVGVVIEATHMCMVMRGVQKMNSKTVTSTMLGVFREDPKTREEFLTLIRS

SEQ ID NO: 63 - DNA, 564 bp, Escherichia coli
atgatcctgcttatagataactacgattcttttacctggaacctctaccagtacttttgtgaactgggggcggatgtgctggttaagcgcaacgatgcgttgacgctggcggatatcgacgcccttaaaccacaaaaagttgtcatctcacccggcccctgtacgccagatgaagccgggatctcccttgacgttattcgccactatgccgggcgcttgccgattcttggcgtctgcctcggtcatcaggcaatggcgcaggcatttggcggtaaagttgtgcgcgccgcaaaggtcatgcacggcaaaacctcgccgattacacataacggtgagggcgtatttcgggggctggcaaatccacttaccgtgacacgctatcattcgctggtagtggaacctgactcgttaccagcgtgctttgaagtgacggcctggagcgaaacccgcgagattatggggattcgccatcgccagtgggatctggaaggtgtgcagttccatccagaaagtattcttagcgaacaaggacatcaactgctggctaatttcctgcatcgctga

SEQ ID NO: 64 - protein, 187 aa, Escherichia coli
MILLIDNYDSFTWNLYQYFCELGADVLVKRNDALTLADIDALKPQKVVISPGPCTPDEAGISLDVIRHYAGRLPILGVCLGHQAMAQAFGGKVVRAAKVMHGKTSPITHNGEGVFRGLANPLTVTRYHSLVVEPDSLPACFEVTAWSETREIMGIRHRQWDLEGVQFHPESILSEQGHQLLANFLHR

SEQ ID NO: 65 - DNA, 1362 bp, Escherichia coli
atgaagacgttatctcccgctgtgattactttaccctggcgtcaggacgccgctgaattgtatttctcccgcttaagccacctgccgtgggcgatgcttttacactccggctatgccgatcatccatatagccgctttgatattgtggtcgccgagccgatttgcactttaaccactttcggtaaagaaaccgttgttagtgaaagcgaaaaacgcacaacgaccactgatgacccgctacaggtgctccagcaggtgctggatcgcgcagacattcgcccagcgcataacgaagatttgccatttcagggcggcgcgctggggttgtttggctacgatctgggccgccgttttgagtcactgccagaaattgcacagcaagatatcgttctgccggatatggctgtgggtatctacgactgggcgctggttgttgaccaccagcgtcaaacagtttctttgctgagtcataatgatgtcaatgctcgtcgggcctggctggaaagccagcaattctcgccgcaggaagatttcacgctcacttccgactggcaatccaatatgacccgcgagcagtacggcgaaaaatttcgccaggtacaggaatatctgcacagcggtgattgctatcaggtgaatctcgcccagcgttttcatgcgacctattctggcgatgaatggcaggcattccttcagcttaatcaggccaaccgcgcgccatttagcgcttttttacgtcttgaacagagtgcaattttaagcctttcgccagagcggtttattctttgtgataatagtgaaatccagacccgcccgattaaaggcacgctaccacgcctgcccgatcctcaggaagatagcaaacaagcagaaaaactggcgaactcagcgaaagatcgtgccgaaaatctgatgattgtcgatttaatgcgtaatgatatcggtcgtgttgccgtagcaggttcggtaaaagtaccagagctgttcgtggtggaacccttccctgccgtgcatcatctggtcagcaccataacggcgcaactaccagaacagttacacgccagcgatctgctgcgcgcggcttttcctggtggctcaataaccggggctccgaaagtacgggctatggaaattatcgacgaactggaaccgcagcgacgcaatgcctggtgcggcagcattggctatttgagcttttgcggcaacatggacaccagcattactatccgcacgctgactgccattaacggacaaatttactgctctgcggggggtggaattgtcgccgatagccaggaagaagcggaatatcaggaaacttttgataaagttaataagatattacgccaactggagaagtaa

SEQ ID NO: 66 - protein, 453 aa, Escherichia coli
MKTLSPAVITLPWRQDAAELYFSRLSHLPWAMLLHSGYADHPYSRFDIVVAEPICTLTTFGKETVVSESEKRTTTTDDPLQVLQQVLDRADIRPAHNEDLPFQGGALGLFGYDLGRRFESLPEIAQQDIVLPDMAVGIYDWALVVDHQRQTVSLLSHNDVNARRAWLESQQFSPQEDFTLTSDWQSNMTREQYGEKFRQVQEYLHSGDCYQVNLAQRFHATYSGDEWQAFLQLNQANRAPFSAFLRLEQSAILSLSPERFILCDNSEIQTRPIKGTLPRLPDPQEDSKQAEKLANSAKDRAENLMIVDLMRNDIGRVAVAGSVKVPELFVVEPFPAVHHLVSTITAQLPEQLHASDLLRAAFPGGSITGAPKVRAMEIIDELEPQRRNAWCGSIGYLSFCGNMDTSITIRTLTAINGQIYCSAGGGIVADSQEEAEYQETFDKVNKILRQLEK

SEQ ID NO: 67 - DNA, 1263 bp, Arabidopsis thaliana
  misc_feature (19)..(19): n is a, c, g, or t
atgaagatgtcaatcgggnaggcatcaaaaagactcctttccgtgtcgccaaggcccnttcgtgaanggaccagaggttataagcaaaaggtgaaggactatgtacagagtgctctgtttccagaagcagggttggatgaaggagttgggcaagcaggaggagtcggaggacttgttgttgtcagagacctcgatcattactcatactgtgaatcttgcttgcttccttttcatgtcaagtgtcacataggttatgtcccatcgggccagagagtgttaggactgagcaagttctctagagtcactgatgttttcgccaagcggctccaagaccctcagcgtttggctgatgatatttgttcagctctccaacattgggtcaaaccagctggagccgctgttgttcttgaatgctctcacattcacttccccagtttggacttggactctctgaacttgtctagccaccgtggatttgtgaagctactggtttcctcggggtcaggagttttcgaggatgaaagctcgaatctttggggtgaatttcagagtttcttgatgttcaaaggtgtaaaaacgcaagctttgtgcagaaatggcagctctgtgaaagagtggtgcccaagcgttaaaagctcgtctaaattatcacctgaagttgacccggaaatggtttctgctgttgtttccatcctgaaatcactgggagaagatccgttgaggaaagaactcattgctacaccaactcgattcctcaaatggatgttgaacttccaaagaaccaacctcgaaatgaagctaaacagctttaaccctgccaaagtcaatggcgaggtcaaagagaaaaggctgcactgtgagctgaacatgcccttctggtcaatgtgtgaacatcatttgcttcctttctatggagttgttcatattggctacttttgtgctgaaggatccaaccccaaccctgttggaagttcactcatgaaagcgattgtacacttttatgggttcaagcttcaagtgcaagagaggatgnctcgacagatcgctnaaacgctatcgcntcttgttggcggggatgtgattgttgtggcggaagctgggcatacttgtatgatctctagaggaattgagaagtttggaagcagcaccgcgacaatcgcagttttgggtcggttttcgagtgacaattccgcaagagcgatgtttctagacaagatccatacaactaatgccttgaagacagagtcaagctctccattttga

SEQ ID NO: 68 - protein, 420 aa, Arabidopsis thaliana
  misc_feature (7)..(7): Xaa can be any naturally occurring amino acid
MKMSIGXASKRLLSVSPRPXREXTRGYKQKVKDYVQSALFPEAGLDEGVGQAGGVGGLVVVRDLDHYSYCESCLLPFHVKCHIGYVPSGQRVLGLSKFSRVTDVFAKRLQDPQRLADDICSALQHWVKPAGAAVVLECSHIHFPSLDLDSLNLSSHRGFVKLLVSSGSGVFEDESSNLWGEFQSFLMFKGVKTQALCRNGSSVKEWCPSVKSSSKLSPEVDPEMVSAVVSILKSLGEDPLRKELIATPTRFLKWMLNFQRTNLEMKLNSFNPAKVNGEVKEKRLHCELNMPFWSMCEHHLLPFYGVVHIGYFCAEGSNPNPVGSSLMKAIVHFYGFKLQVQERMXRQIAXTLSXLVGGDVIVVAEAGHTCMISRGIEKFGSSTATIAVLGRFSSDNSARAMFLDKIHTTNALKTESSSPF

SEQ ID NO: 69 - DNA, 1104 bp, Oryza sativa
atggatcttttctcatactgtgagtcatgcttacttccattcagcatacaattccatgttggctatgtgccctctggtggaagggttgttgggttaagcaagctttcgagagtagctgatgtcttcgccaagaggttgcagaatcctcaaagactggctagtgaagtttgtggtgcattgcatgctagcatacaacctgctggtgtggctgttgctctgcaatgttggcacatacctttgccagaaaacttgaaatgcaagactttgcaaggttggattagcacttcacattcatctcgctctggagtttttgagggtgagagcagctctttttggaatgacttctcagcccttcttaagcttaggggcatagacatggagagggacagccattctgcctccatagcttggtgccctttaaggtctcatgatgtcccagtctgcaatgggcactgcaagaaggctacaaccaacggtgcaatttcacccaaatcagtaccagctccctctaatatggtttctgctgttagctcaatgctcttatcccttggagaggatcccttcaggaaagaacttgtaggtactcctcagcgttacgtgcaatggctgatgaagttcagagcatgtaacctagatgtgaagctgaatggctttacactcaataatttgagtgtataccagagtccagctggagatgctgctgaccatcgagcaatccattctgagctgcatttgccattttgtgcgcagtgcgagcaccatcttctgccgttctatggagtagtgcatattggctaccttgacggcggagatggtgaagtgattgatcgatctcattttcaggccttggttcatttttatggatgcaagcttcaggttcaagagagaatgacaaggcagatagctgaagcagtttattctgtttcgcattgtggggccatagttgttgtagaagctaaccacatttgcatgatatcaaggggaatagagaaaatcaggagtagcactgcaacgattgcagttctgggtcagtttttgacggacccttctgccaaggcacgctttctgcagaacgtagtagatacaactggtttggcagtatga

SEQ ID NO: 70 - protein, 367 aa, Oryza sativa
MDLFSYCESCLLPFSIQFHVGYVPSGGRVVGLSKLSRVADVFAKRLQNPQRLASEVCGALHASIQPAGVAVALQCWHIPLPENLKCKTLQGWISTSHSSRSGVFEGESSSFWNDFSALLKLRGIDMERDSHSASIAWCPLRSHDVPVCNGHCKKATTNGAISPKSVPAPSNMVSAVSSMLLSLGEDPFRKELVGTPQRYVQWLMKFRACNLDVKLNGFTLNNLSVYQSPAGDAADHRAIHSELHLPFCAQCEHHLLPFYGVVHIGYLDGGDGEVIDRSHFQALVHFYGCKLQVQERMTRQIAEAVYSVSHCGAIVVVEANHICMISRGIEKIRSSTATIAVLGQFLTDPSAKARFLQNVVDTTGLAV

SEQ ID NO: 71 - DNA, 1479 bp, Zea mays
cttgcgatacgattcataccgcagaaccaacagaatccaaaacattctgtaagaagcacgccttggcagaagggtcggccaaaaactggcccaaaactgcaattgttgccgtgttgctccttattttctctatcccccttgagatcatgcaaatgtggttggcctcaacaacgaccatggccccattgtgcgaaactgaataaaccgcttcagctatctgccttgtcatcctttcttgaacctgaagcttgcacccgtagaaatgaaccaacgcctgaaaatgcgaccgatcaatgccttctccgcttccattgccaaagtacccaatgtgaactactccgtagaagggcagaagatggtgttcacattgagcacaaaatggcaagtgcagctccgatctgattgctccatgatcagttgtgcctccacctgtcctctcatataagctaacattactaagtgtaaaaccattcagcttcacgtctagtagattacatgctttgaacttcatcagccactgcacataacgctgaggagtgcctaaaagctctttcctgagggggtcctctccgagtgataagagcatcgaggtaacagcagaaaccatgccattagttgagtttttcttgcagagcccgttagagagtggaacctcatgagaccttaaagggcaccaaggaatagaaacagtacggtccttagcctccacgtctatgcccctaagcttaacaagagccaagaagtcactccagaaagtgctgctttcaccttcaaagactccagagcgagatgaatgtgaagttctaatccaaccttccaaagtcttgcattccaagttttctggtaaaggaatgtgccagcactgcagagcaacagccacaccagcaggttgaatgctagcatgcagtgcaccacagatttcattagctagtctttgagggttttgcaatctcttggcaaagacatcagatactctagaaagcttgcttaacccaaccactcttccacctgagggaacatacccgacatggcactgtatgctgaatggaagcaagcatgactcacaataggagaaaagttcaatgtctcgaacaactacttgcccgccagttccaccagcagatccagtccttttatccacaccaacctctggaaacagagcaccttgcactatgtcttttactttttgcctgtagcctgtagatttagatatatgttaaatacagatcatgaagcaattaaaacaaaagtagagatgatgaataagaccccacatatcttcattaaaacaaaaggttatgcaaaaaaaaacatcctcacatatcacatatgtcactcttccttgtataaacagttctgtctctttgaataagaacaccatgacaacctctactacacatgacttgcatgggaatagattatccagatttcacatgacttctttataatagaacaagaatgatgtggctccgtgtcaga

SEQ ID NO: 72 - DNA, 668 bp, Bos taurus
atggcccaggcccctagaactccaagggccaggacagaccttcttaatgtctgcatggatgccaagcaccacaaggcagaaccaggcccagaggactctcttcatgagcagtgcagcccttggaggaagaatgcctgctgctctgtcaacaccagcatagaagcccataaggacatttcttacctttacagattcaactgggaccactgcggcaagatggagcctgcttgcaagaggcactttattcaggacacctgtctctacgagtgctcacctaatcttggcccttggatcagggaggttaaccagaggtggaggaaagagagggtgcttggtgtgcctctttgcaaagaggactgtcagagctggtgggaagactgcaggacctcttacacctgcaagagcaactggcacaagggctggaactggacctcaggctacaaccagtgcccagtgaaagctgcctgccacaggtttgacttctacttcccaactcctgctgctctttgcaatgaaatctggtctcactcttacaaggtcagcaattacagcaggggcagcggcaggtgcattcagatgtggttcgaccctttccagggcaaccctaatgaggaggtggcaagattttatgctgagaaccctacttctggctctacacctcagggcat

SEQ ID NO: 73 - protein, 223 aa, Bos taurus
MAQAPRTPRARTDLLNVCMDAKHHKAEPGPEDSLHEQCSPWRKNACCSVNTSIEAHKDISYLYRFNWDHCGKMEPACKRHFIQDTCLYECSPNLGPWIREVNQRWRKERVLGVPLCKEDCQSWWEDCRTSYTCKSNWHKGWNWTSGYNQCPVKAAHCRFDFYFPTPAALCNEIWSHSYKVSNYSRGSGRCIQMWFDPFQGNPNEEVARFYAENPTSGSTPQGI

(19) United States
(12) Patent Application Publication
(10) Pub. No.: US 2010/0183750 A1
(43) Pub. Date: Jul. 22, 2010

(54) FORTIFICATION OF PLANTS WITH FOLATES BY METABOLIC ENGINEERING

(75) Inventors: Dominique Van Der Straeten, Gent (BE); Serguei Storojenko, Gent (BE); Willy Lambert, Gent (BE); Guo-Fang Zhang, Cleveland, OH (US)

Correspondence Address: KNOBBE MARTENS OLSON & BEAR LLP, 2040 MAIN STREET, FOURTEENTH FLOOR, IRVINE, CA, 92614, US

(73) Assignee: Universiteit Gent, Gent (BE)

(21) Appl. No.: 12/664,992

(22) PCT Filed: Jun. 19, 2008
(86) PCT No.: PCT/EP08/57746

§ 371 Date: Dec. 16, 2009

(30) Foreign Application Priority Data
Jun. 22, 2007 (EP) 07110852.6

(51) Int. Cl. C12N 15/82; A01H 5/00; A01H 5/10; C12N 15/29; C12N 5/10; A61K 36/00; A23K 1/18

(52) U.S. Cl. 424/725; 800/298; 800/278; 536/23.2; 435/320.1; 435/419; 426/2

(57) ABSTRACT

The invention relates to a transgenic plant overexpressing a polynucleotide encoding a plant GTP cyclohydrolase I (GTPCHI) enzyme. The present invention also relates to a product comprising the transgenic plant of the invention or a harvestable part, plant organ, plant tissue, plant cell, seed, or a propagule of said plant. The present invention further elucidate the use of the plant of the invention to cross breed with other plants, as biofortification to enrich the quality of the food intake of an animal or human subject, for the manufacture of a medicament to prevent folate insufficiency, or, for the treatment of folate insufficiency or diseases or disorders resulting from said insufficiency in an animal or human subject.

### TECHNICAL FIELD

The present invention relates to methods for the fortification of plants with folates by metabolic engineering. In particular, the present invention relates to a vitamin-enhanced crop, which can be used to, for instance, alleviate vitamin deficiency.

### BACKGROUND ART

Folate is the generic name of a natural water-soluble B vitamin (B9) represented by a family of structurally related interconvertible enzyme co-factors. Folates play a role as donors and acceptors of one-carbon (C1) units in a complex set of reactions termed C1 metabolism, the central part of which is represented by DNA biosynthesis and the methylation cycle. Folates are tripartite molecules containing a pterin moiety, para-aminobenzoic acid (p-ABA) and one or several glutamate residues (FIG. 1A). In plants, pterin precursors are synthesized from GTP in the cytosol (pteridin or pterin branch) while p-ABA is derived from chorismate in plastids (p-ABA branch). Both pterin precursors and p-ABA are imported in the mitochondria to participate in the condensation to folates (FIG. 1B).

Folate deficiency results in serious health problems, including neural tube defects (NTD) as spina bifida in infants, megaloblastic anemia, and several neurodegenerative disorders as Alzheimer's disease. It is also connected to a higher risk of cardio-vascular disease and development of a range of cancers. In case of NTD prevention, the major problem is that the neural tube is formed between days 21-27 after conception, before most women realize they are pregnant. Thus, in order to prevent NTD, women should take high amounts of folate from the pen-conceptional phase until 12 weeks of gestation. Adequate dietary folate intake can prevent onset of these conditions. Even in the developed world folate deficiency is a widespread phenomenon.

Humans and animals cannot synthesize folates on their own; therefore, they have to rely on plant food as main source of the vitamin. The Recommended Daily Allowance (RDA) for folates for an adult person is 400 μg and even higher (600 μg) for pregnant women (National Institutes of Health. Office of Dietary Supplements. Dietary Supplement Fact Sheet: Folate. http://ods.od.nih.gov/factsheets/folate.asp). Some widely consumed plant foods are extremely poor in folates, which results in world-wide folate deficiency. Folate levels in different plant foods vary considerably and important staple crops such as tomato, maize, rice and other cereals are poor in folate (see Table 1) (USDA National Nutrient Database for Standard Reference http://www.nal.usda.gov/fnic/foodcomp/search/).

Because of the low compliance of taking folic acid supplements, some countries (e.g. USA, Canada) have introduced mandatory fortification of flour with folic acid. However, this is not the case for Europe, where the sufficiency in folate supply relies mainly on diet diversification and supplementation (folate pills), with very limited success in improving the folate status of the population. Although compulsory food folate fortification (with synthetic folic acid) programs combined with folate pill distribution campaigns have improved the situation in some countries, these approaches are difficult to implement in the developing world since they require specialized infrastructure and can hardly reach remote areas where folate deficiency is most dramatic. Biofortification of major crops by means of biotechnology provides a rational alternative or at least a complementary solution to folate malnutrition.

Hanson's group recently reported successful folate enhancement in tomato fruit (Diaz de la Garza et al., 2007, Proc. Natl. Acad. Sci. USA 104:4218-4222). This was achieved by crossing tomato plants overexpressing mammalian GTP cyclohydrolase I (GTPCHI) (Diaz de la Garza, R. D. et al., 2004, Proc. Natl. Acad. Sci. USA 101:13720-13725) with plants overexpressing aminodeoxychorismate synthase (ADCS) from Arabidopsis (FIG. 1B), in fruits. Since it is accepted that the activity of plant GTPCHI is subject to negative feedback regulation, Diaz de la Garza et al. used a mammalian GTPCHI to avoid this negative control (see also WO 2006/034501). Folates in these transgenic tomatoes were enhanced up to 25-fold. Storozhenko et al. (Trends in food science and technology. Elsevier Science Publishers GB 2005, 16:271-281) and Dellapenna (PNAS 2007, 104:3675-3676) refer to the enhancement of folate in food or staple crops.

Hanson et al. (WO 2006/034501, Diaz de la Garza et al., 2004, Proc. Natl. Acad. Sci. USA 101:13720-13725;) and other groups (Hossain et al., 2004, Proc. Natl. Acad. Sci. USA 101:5158-5163) also reported the overexpression of the feedback-insensitive bacterial and mammalian GTPCHI combined with exogenous application of pABA to increase fotate production in Arabidopsis or tomato, respectively. This combination allowed the folate content to increase with a factor of 4 and 10, respectively.

### OBJECTS OF THE INVENTION

The present invention aims at further increasing the folate level in plants so that the recommended dietary allowance (RDA) value is more easily reached in a single serving. The present inventors focused on the folate enhancement in rice as a model, as rice is one of the major staple crops consumed in developing countries, and to a lesser extent in the developed world. Grain crop consuming populations in developing countries often live in a condition of persistent folate deficiency. Furthermore, in contrast to tomato fruit, rice is easy to store and can be distributed to remote regions. However, the average folate contents in cereals is very low, particularly in rice, rendering the problem of folate deficiency most prominent in those regions where people depend on cereal staples. One hundred grams of cooked white rice contain approximately 1-3 μg of folates, which is 400 times lower than the RDA value. Hence, in rural areas of certain provinces of China, the incidence of NTD was reported to be around 7 per 1,000 live births. This rate is among the highest in the world and is approximately ten-fold higher than rates in western surveillance systems. Similar high incidence of NTD was reported in certain regions in India.

The present invention provides methods which may be used to increase the folate content in plants. Although the concept is proven for rice, the concept found may easily be applied to other crops.

### DETAILED DESCRIPTION OF THE INVENTION

The present invention relates to a method wherein a polynucleotide encoding a plant enzyme that catalyzes the synthesis of dihydroneopterin-PPP from GTP is overexpressed in a plant. According to the present invention, said method may comprise incorporating in a plant a polynucleotide encoding an enzyme that catalyzes the synthesis of dihydroneopterin-PPP from GTP, wherein said enzyme is a plant enzyme. According to the present invention, said method may comprise incorporating in a plant a polynucleotide encoding an enzyme that catalyzes the synthesis of dihydroneopterin-PPP from GTP, wherein said enzyme is under feedback control of the plant. Said enzyme may be the plant GTP cyclohydrolase I (GTPCHI) enzyme. The term “polynucleotide” refers to a single stranded or double stranded nucleic acid sequence, said nucleic acid may consist of deoxyribonucleotides or ribonucleotides or may be amplified cDNA or amplified genomic DNA.

With ‘feed-back control’ is meant that the enzyme activity is under control of metabolic compounds of the folate synthesis pathway. In contrast to the plant GTPCHI, the mammalian and bacterial GTPCHI are not under a feed-back control regulating the GTPCHI activity and are thus not subject to metabolic regulation in a plant cell.

In contrast to the approaches described by Hanson's and other groups, the present invention uses genes or enzymes catalyzing the synthesis of dihydroneopterin-PPP from GTP derived from plants. Before the invention was made, the use of said plant enzymes or genes to increase folate in plants was inconceivable as they are submitted to feed-back control. That is also the reason why before the present invention the feedback sentivive GTPCHI of plant has never been overexpressed in plant in order to increase the folate content in plant.

Unexpectedly, the overexpression of the plant GTPCHI in combination with overexpression of a plant gene encoding amino-deoxychorismate synthase (ADCS) allows to further increase the folate content in plant, even up to 100 times compared to the folate content present in wild type plants. Using said method the highest folate content reported for plant species thus far could be obtained. The biofortified plants of the invention can thus easily satisfy the daily folate requirement for an average adult person and even that of a pregnant woman. However, it is not excluded that when analyzing more transgenic plants, produced by the method of the present invention, plants may be found with even higher folate levels. As indicated above, the unexpected folate increase could be obtained by overexpressing plant GTPCHI and plant ADCS in plant, however, this increase may also be obtained when overexpressing other members of the folate pathway together with plant GTPCHI.

Furthermore, in the transgenic plants of the present invention, 5-methyltetra-hydrofolate was found to be the major folate present. Almost 90% of the folate content is 5-methyltetrahydrofolate, which makes the transgenic plant of the present invention superior. Indeed, 5-methyltetrahydrofolate has higher efficiency in increasing red blood cell folate concentration as compared to folic acid and 5-methyltetrahydrofolate does not mask symptoms of vitamine B12 deficiency as high folic acid consumption does.

In addition, in the transgenic plants of the invention, 4.5-10 times lower p-ABA levels and 80-fold lower pterin intermediates levels were found compared to folate engineered tomato described by Hanson's group. Indeed, the approach of Hanson's group leads to excessive accumulation of folate intermediates most probably due to the loss of intrinsic regulation of the pathway. Hanson found that while folates in these transgenic tomatoes were enhanced up to 25-fold, the levels of pteridine intermediates as well as p-ABA were also greatly elevated (>20-fold). Furthermore, there are clear evidences that a misbalance of in particular pteridine status in man may have serious consequences for human health. Therefore, it is of primary importance to provide crops with low levels of intermediates of said folate synthesis pathway. Thus also for this problem, the present invention provides a solution. The use of a plant GTPCHI allows the optimal flux of pteridine precursors and p-ABA through the pathway towards folates. Up to 100 times higher folate contents were found in transgenic lines of the present invention as compared to the wild type, without substantial accumulation of folate biosynthesis intermediates, rendering the biofortified plant of the present invention perfectly safe, with regard to concerns of for instance pterin over-consumption.

The present invention further teaches that, the use of plant GTPCHI, which retains its intrinsic negative feedback regulation, in combination with for instance plant ADCS results in a balanced tuning of both enzyme activities, and allows optimal flux of pteridine precursors and p-ABA through the pathway towards folates. In this way folate biofortified plants of the present invention contain much less of both pABA and pteridines as compared to the engineered tomato expressing the mammalian GTPCHI. Contrarily, as shown by Hanson's group, overexpression of mammalian feedback-free GTPCHI and plant ADCS results in very high p-ABA and pteridine accumulation. Indeed, the molar ratios of folates/pABA/pterins in folate enhanced tomatoes roughly equals to 1/2.5/0.75, while being 1/0.5/0.013 in the biofortified plants of the present invention.

The advantage of the present invention is that the provision of crops with further increase of folate content eliminates the need to additional intake of folate pills. This approach also avoids the need for infrastructure for fortification of flour. Furthermore, there is applicability over a wide crop range. The strategy of the present invention uses the plant genes, allowing intrinsic (feedback/feedforward) regulation to occur; hence not leading to excessive intermediate accumulation and thus avoiding potential health implications.

According to the present invention, the polynucleotide encoding the plant enzyme catalyzing the synthesis of dihydroneopterin-PPP from GTP may be derived from the same plant in which folate increase is aimed at, or may be derived from related or non-related plant species. The plants of the present invention show an increased plant GTPCHI activity. According to the present invention, the plant GTPCHI enzyme may for instance be derived from Arabidopsis (thaliana) (SEQ ID NO:2 and SEQ ID NO:68), tomato (AY069920) (SEQ ID NO:4), rice (J023007K22) (SEQ ID NO:6) (Os04g56710) (SEQ ID NO:70), wheat (TA71893_{—}4565) (SEQ ID NO:8), or maize (AZM5_{—}146121) (SEQ ID NO:10). The sequence of the rice GTPCHI enzyme, deposited as Os04g56710, lacks the 5′ end of the rice enzyme deposited in J023007K22 (see also SEQ ID NO:69). SEQ ID NO:71 discloses the reverse complement of SEQ ID NO:9. Furthermore, said plant GTPCHI enzyme may comprise the amino acid sequence chosen from the group consisting of a sequence represented by SEQ ID NO:2, 4, 6, 8, 10, 68 and 70, an analogue, a homologue, and, an enzymatically functional fragment of any of SEQ ID NO:2, 4, 6, 8, 10, 68 or 70, wherein said analogue or homologue is at least 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% identical to the amino acid sequence represented by any of SEQ ID NO:2, 4, 6, 8, 10, 68 or 70. The activity of GTPCHI, the analogue, the homologue or the fragment, can be determined by a spectrophotometric based kinetic assay, wherein the rate of increase of A₃₄₀ over a 1 h-period at given intervals at 37° C. is used as a measure for the accumulation of dihydroneopterin triphosphate (DHNPPP), the product of GTPCHI, in a reaction mixture containing its substrate, GTP, in a phosphate buffered solution (Kolinsky and Gross JBC 2004, 279:40677-40682).

In the present context, the term “homologue polypeptide” refers to those polypeptides, enzymes or proteins which have a similar activity, notwithstanding any amino acid substitutions, additions or deletions thereto. A homologue may be isolated or derived from the same or another species. Furthermore, the amino acids of a homologous polypeptide may be replaced by other amino acids having similar properties, for example hydrophobicity, hydrophilicity, hydrophobic moment, charge or antigenicity, and so on. The term “analogue polypeptide” encompasses proteins and polypeptides as hereinbefore defined, notwithstanding the occurrence of any non-naturally occurring amino acid analogues therein. Substitutions encompass amino acid alterations in which an amino acid is replaced with a different naturally-occurring or a non-conventional amino acid residue. Such substitutions may be classified as “conservative”, in which case an amino acid residue contained in a polypeptide is replaced with another naturally-occurring amino acid of similar character, for example Gly⇄Ala, Val⇄Ile⇄Leu, Asp⇄Glu, Lys⇄Arg, Asn⇄Gln or Phe⇄Trp⇄Tyr. Substitutions encompassed by the present invention may also be “non-conservative”, in which an amino acid residue which is present in a polypeptide is substituted with an amino acid having different properties, such as a naturally-occurring amino acid from a different group (e.g. substituted a charged or hydrophobic amino acid with alanine), or alternatively, in which a naturally-occurring amino acid is substituted with a non-conventional amino acid. Amino acid substitutions are typically of single residues, but may be of multiple residues, either clustered or dispersed.

As known in the art, “similarity” between two polypeptides is determined by comparing the amino acid sequence and its conserved amino acid substitutes of one polypeptide to the sequence of a second polypeptide. Moreover, also known in the art is “identity” which means the degree of sequence relatedness between two polypeptide or two polynucleotide sequences as determined by the identity of the match between two strings of such sequences. Both identity and similarity can be readily calculated. While there exist a number of methods to measure identity and similarity between two polynucleotide or polypeptide sequences, the terms “identity” and “similarity” are well known to skilled artisans (Carillo and Lipton, 1988 SIAM J. Applied Math. 48:1073). Methods commonly employed to determine identity or similarity between two sequences include, but are not limited to, those disclosed in “Guide to Huge Computers” (Martin J. Bishop, ed., Academic Press, San Diego, 1994) and Carillo and Lipton (1988 SIAM J. Applied Math. 48:1073). Preferred methods to determine identity are designed to give the largest match between the two sequences tested. Methods to determine identity and similarity are codified in computer programs. Preferred computer program methods to determine identity and similarity between two sequences include, but are not limited to, GCG program package (University of Wisconsin, U.S.A.; Devereux et al., 1984, Nucleic Acids Research 12:387), BLASTP, BLASTN and FASTA (Altschul et al., 1990, J. Mol. Biol. 215:403-410).

In the method of the present invention, a polynucleotide encoding at least a plant enzyme that catalyzes the synthesis of dihydropterin-PPP from GTP may be ‘incorporated’ into a plant. A genetic construct or polynucleotide may be incorporated or introduced into a plant, thereby producing a ‘transgenic plant’, by various techniques known to those skilled in the art. The technique used for a given plant species or specific type of plant tissue depends on the known successful techniques. Means for introducing recombinant DNA into plant tissue include, but are not limited to, direct uptake into protoplasts (Kerns et al., 1982, Nature 296:72-74; Paszkowski et al., 1984, EMBO J. 3:2717-2722), PEG-mediated uptake to protoplasts (Armstrong et al., 1990, Plant Cell Reports 9:335-339) microparticle bombardment electroporation (Fromm et al., 1985, Proc. Natl. Acad. Sci USA 82:5824-58-28), microinjection of DNA (Crossway et al., 1986, Mol. Gen. Genet. 202:179-185), micro bombardment of tissue explants or cells (Christou et al., 1988, Plant. Physiol. 87:671-674; Sanford, 1988, Particulate Science and Technology 5:27-37) or T-DNA mediated transformation from Agrobacterium to plant tissue. Methods for the Agrobacterium-mediated transformation of plants will be well-known to those skilled in the art. In particular, methods for Agrobacterium-mediated transformation of rice (Oryza sativa) tissue have been disclosed by Hiei et al. (1994, Plant J. 6:271-282). Representative T-DNA vectors systems are described in the following references: An et al., 1985, EMBO J. 4:277-284); Herrera-Estrella et al., 1983, Nature 303:209-213; Herrera-Estrella et al., 1983, EMBO J. 2:987-995; Herrera-Estrella et al., 1985, In Plant genetic Engineering, Cambridge University Press, NY, p. 63-93). A transgenic plant is thus distinct from a wild type plant by the presence of (a) transgene sequence(s), be it of homologous or heterologous origin, the length of said transgene sequence(s) may vary between 10 to thousands of polynucleotides. For instance, the transgenic plant of the present invention may be changed in it polynucleotide sequence so that the polynucleotide encoding a plant GTP cyclohydrolase I (GTPCHI) is overexpressed.

The present invention further indicates that the plant GTPCHI enzyme used in the method of the present invention may be encoded by a polynucleotide comprising the polynucleotide sequence chosen from the group consisting of a sequence represented by SEQ ID NO:1, 3, 5, 7, 9, 67, 69 and 71 or a polynucleotide sequence that hybridizes under stringent conditions with a polynucleotide sequence complementary to the sequence chosen from the group consisting of a sequence represented by SEQ ID NO:1, 3, 5, 7, 9, 67, 69 and 71. With the expression ‘hybridizes under stringent conditions’ is meant hybridizing to the sequence under conditions such as the ones described by Sambrook et al., Molecular Cloning, Laboratory Manuel, Cold Spring, Harbor Laboratory press, New York. An example of “stringent hybridization conditions” is as follows: hybridize in 50% formamide, 5×SSC, 50 mM sodium phosphate (pH 6.8), 0.1% sodium pyrophosphate, 5×Denhardt's solution, 50 μg/ml sonicated salmon sperm DNA, 0.1% SDS and 10% dextran sulfate at 42° C.; and wash at 42° C. (or higher, e.g., up to two degrees C. below the Tₘ of the perfect complement of the probe sequence) in 0.2×SSC and 0.1% SDS. The plant GTPCHI enzyme used in the method of the present invention may be also be defined as being encoded by a polynucleotide comprising the polynucleotide sequence chosen from the group consisting of a sequence represented by SEQ ID NO:1, 3, 5, 7, 9, 67, 69 and 71, an analogue and a homologue of any of SEQ ID NO:1, 3, 5, 7, 9, 67, 69 or 71, wherein said analogue or homologue is at least 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% identical to the polynucleotide sequence represented by any of SEQ ID NO:1, 3, 5, 7, 9, 67, 69 or 71.

The present invention indicates that in the method of the present invention the plant, overexpressing the enzyme that catalyzes the synthesis of dihydroneopterin-PPP from GTP, may be grown in the presence of p-aminobenzoate (PABA). Alternatively, in the method of the present invention, said plant may be engineered to express increased levels of PABA. For instance, said plant can be made transgenic for, or transformed with, one or more polynucleotide molecules encoding one or more enzymes that catalyze synthesis of PABA. According to the present invention, said one or more enzymes may be 4-amino-4-deoxychorismate synthase (ADCS) and/or 4-amino-4-deoxychorismate lyase (ADCL). Said plant may thus further overexpress a polynucleotide encoding a plant 4-amino-4-deoxychorismate synthase (ADCS) and/or 4-amino-4-deoxychorismate lyase (ADCL). The present invention indicates that said ADCS and/or ADCL enzymes may be of plant origin.

According to the invention, the 4-amino-4-deoxychorismate synthase (ADCS) enzyme may be derived from Arabidopsis (thaliana) (SEQ ID NO:12), tomato (AY425708) (SEQ ID NO:14) or rice (Os06g48620) (SEQ ID NO:16); and/or may comprise the amino acid sequence chosen from the group consisting of a sequence represented by SEQ ID NO:12, 14, and 16, an analogue, a homologue, and, an enzymatically functional fragment of any of SEQ ID NO:12, 14, or 16, wherein said analogue or homologue is at least 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% identical to the amino acid sequence represented by any of SEQ ID NO:12, 14, or 16; wherein said enzymatically functional fragment may be chosen from the group consisting of the putative chloroplastic transit peptide domain (1-87 aa of the polypeptide represented by SEQ ID NO:12 or corresponding domains in SEQ ID NO:14 or 16), the putative Pab A domain (88-334 aa of the polypeptide represented by SEQ ID NO:12 or corresponding domains in SEQ ID NO:14 or 16), and the putative Pab B domain (430-919 aa of the polypeptide represented by SEQ ID NO:12 or corresponding domains in SEQ ID NO:14 or 16). The term ‘aa’ indicates the position of the amino acids, wherein the numbering starts from AA1=Methionine determined by the start of the open reading frame. The proposed functional domains are deducted by comparison with yeast and bacterial enzymes, the position of the real domains for the plant enzymes may thus slightly deviate from the indicated positions. It is also not excluded that the corresponding yeast or bacterial enzymes and/or coding sequences may be used. As the enzymatic activity may be easily be determined by a skilled person in the art, a skilled person may easily analyze which domain of said enzyme may be used to replace the full length enzyme. This also applies for the other enzymes used in the methods of the present invention. A standard ADCS activity assay contains 100 mM Tris-HCl buffer pH8, 5 mM MgCl₂, 0.01-5 mM L-glutamine, and 0-50 μM chorismate as free acid or barium salt and a desalted extract from a pabA⁻pabB⁻ E. coli strain containing PabC activity, together with the extract harboring the ADCS enzyme, running for 20-30 min at 37° C., followed by HPLC-based measurement of pABA as described by Sahr et al 2006, Biochem J. 396:157-162. The present invention further indicates that in the method of the present invention the ADCS enzyme may be encoded by a polynucleotide comprising the polynucleotide sequence chosen from the group consisting of a sequence represented by SEQ ID NO:11, 13, and 15 or a polynucleotide sequence that hybridizes under stringent conditions with a polynucleotide sequence complementary to the sequence chosen from the group consisting of a sequence represented by SEQ ID NO:11, 13, and 15. The ADCS enzyme used in the method of the present invention may be also be defined as being encoded by a polynucleotide comprising the polynucleotide sequence chosen from the group consisting of a sequence represented by SEQ ID NO:11, 13 and 15, an analogue and a homologue of any of SEQ ID NO:11, 13 or 15, wherein said analogue or homologue is at least 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% identical to the polynucleotide sequence represented by any of SEQ ID NO:11, 13 or 15.

In the method of the present invention, the 4-amino-4-deoxychorismate lyase (ADCL) enzyme may derived from Arabidopsis (thaliana (SEQ ID NO:18)), tomato (AY547289) (SEQ ID NO:20) or rice (Os02g17330) (SEQ ID NO:22). Furthermore, said ADCL enzyme may comprise the amino acid sequence chosen from the group consisting of a sequence represented by SEQ ID NO:18, 20, and 22, an analogue, a homologue, and, an enzymatically functional fragment of any of SEQ ID NO: 18, 20 or 22, wherein said analogue or homologue is at least 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75% 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% identical to the amino acid sequence represented by any of SEQ ID NO: 18, 20 or 22; and wherein said enzymatically functional fragment may be chosen from the group consisting of the putative chloroplastic transit peptide domain (1-72 aa of the polypeptide represented by SEQ ID NO:18 or corresponding domains in SEQ ID NO:20 or 22) and the enzymatically active fragment (Pab C) domain (73-373 aa of the polypeptide represented by SEQ ID NO: 18 or corresponding domains in SEQ ID NO:20 or 22). The activity of the Pab C fragment has been experimentally proven. Said ADCL enzyme may be encoded by a polynucleotide comprising the polynucleotide sequence chosen from the group consisting of a sequence represented by SEQ ID NO:17, 19 and 21, or a polynucleotide sequence that hybridizes under stringent conditions with a polynucleotide sequence complementary to the sequence chosen from the group consisting of a sequence represented by any of SEQ ID NO: 17, 19 or 21. The ADCL enzyme used in the method of the present invention may be also be defined as being encoded by a polynucleotide comprising the polynucleotide sequence chosen from the group consisting of a sequence represented by SEQ ID NO:17, 19 and 21, an analogue and a homologue of any of SEQ ID NO: 17, 19 or 21, wherein said analogue or homologue is at least 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% identical to the polynucleotide sequence represented by any of SEQ ID NO: 17, 19 or 21. A standard ADCL activity assay contains 100 mM Tris-HCl buffer pH8, 5 mM MgCl₂, 0.01-5 mM L-glutamine, and 0-50 μM chorismate as free acid or barium salt and a desalted extract from a pabC⁻E. coli strain containing PabA and PabB activities, together with the extract harboring the ADCL enzyme, running for 20-30 min at 37° C., followed by HPLC-based measurement of pABA as described by Sahr et al 2006 Biochem J. 396:157-162.

The present invention thus relates for instance to a method for increasing folate levels in a plant, said method comprising incorporating in a plant a polynucleotide encoding the GTPCHI enzyme derived from Arabidopsis thaliana represented by SEQ ID NO:1 or SEQ ID NO:67 and a polynucleotide encoding the 4-amino-4-deoxychorismate synthase derived from Arabidopsis thaliana represented by SEQ ID NO:11 and/or a polynucleotide encoding the 4-amino-4-deoxychorismate lyase derived from Arabidopsis thaliana represented by SEQ ID NO:17.

The present invention further suggests that in the method of the present invention, the plant may be further engineered to express other enzymes for synthesizing folate in plants such as enzymes chosen from the group consisting of DHNA (dihydroneopterin aldolase) (SEQ ID NO:46), HPPK/DHPS (hydroxymethyldihydropterin pyrophosphokinase/dihydropteroate synthetase) (SEQ ID NO:42), DHFS (dihydrofolate synthase) (SEQ ID NO:44), DHFR (dihydrofolate reductase) (SEQ ID NO:48, 50, 52) and FPGS (folylpolyglutamyl synthase) (SEQ ID NO:54, 56, 58) or a combination thereof. Furthermore, said enzyme may comprise the amino acid sequence chosen from the group consisting of a sequence represented by SEQ ID NO: 42, 46, 44, 48, 50, 52, 54, 56 and 58, an analogue, a homologue, and, an enzymatically functional fragment of any of SEQ ID NO: 42, 44, 46, 48, 50, 52, 54, 56, or 58, wherein said analogue or homologue is at least 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% identical to the amino acid sequence represented by any of SEQ ID NO: 42, 46, 44, 48, 50, 52, 54, 56 or 58. Said enzymes may be also be defined as being encoded by a polynucleotide comprising the polynucleotide sequence chosen from the group consisting of a sequence represented by SEQ ID NO:41, 43, 45, 47, 49, 51, 53, 55 and 57, an analogue and a homologue of any of SEQ ID NO: 41, 43, 45, 47, 49, 51, 53, 55 or 57, wherein said analogue or homologue is at least 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% identical to the polynucleotide sequence represented by any of SEQ ID NO: 41, 43, 45, 47, 49, 51, 53, 55 or 57. In addition, in the method of the present invention, the plant may be further engineered to relocate the synthesis of the folate to the cytosol; to express transporters of folate precursors and/or to improve folate stabilization such as expressing folate binding proteins (FBP). A large number of mammalian (but not plant) FBP genes have been described to date (Henderson, 1990, Ann Rev Nutrition 10: 319-335). For engineering of plants, human FBP (BT007158), cow's milk FBP (PO₂₇₀₂), or the unrelated rat liver (NM_{—}017084), human (NM_{—}018960), pig (D13308) FBP, Glycine N-methyltransferase (GNMT) may be used. In addition, a synthetic FBP encoding cow's milk FBP (P02702), with adjusted codon bias to fit the codon usage of rice storage proteins (glutelin, globulin, prolamines), ensuring optimal translation efficiency in the endosperm, may be used (SEQ ID NO:72 and SEQ ID NO:73). The latter may be translationally fused to a carrier-protein, to improve protein stability (Streatfield, 2007, Plant Biotechnology Journal 5: 2-15). As a carrier-protein, endosperm storage proteins can be used (Yang et al., 2007, Plant Biotechnology Journal 5: 815-826), or cytosolic carbonic anhydrase, one of the most abundant plant proteins (Fabre et al., 2007, Plant Cell Environ. 30: 617-629).

The above-mentioned enzymes may be produced in plant by placing the coding regions for said enzymes under control of a plant expressible promoter sequence. Reference herein to a ‘promoter’ is to be taken in its broadest context and includes the transcriptional regulatory sequences of a classical eukaryotic genomic gene, including the TATA box which is required for accurate transcription initiation, with or without a CCAAT box sequence and additional regulatory sequences (i.e. upstream activating sequences, enhancers and silencers) which alter gene expression in response to developmental and/or external stimuli, or in a tissue specific manner. A promoter is usually, but not necessarily, positioned upstream of 5′, of a structural gene, the expression of which it regulates. Furthermore, the regulatory elements comprising a promoter are usually positioned within 2 kb of the start site of transcription of the genetic sequence which encodes the protein. According to the present invention, said plant expressible promoter sequence may be chosen from a group consisting of a constitutive promoter, a root specific promoter, a leaf specific promoter, a stem specific promoter, an endosperm specific promoter, a flower specific promoter, a seed specific promoter, a fruit specific promoter, a tuber specific promoter and a bulb specific promoter. The choice which promoter to use depends on in which part of the plant folate increase is aimed at. When choosing for a an endosperm-specific promoter, said endosperm promoter may be chosen from the group consisting of a Glutelin promoter, a Globulin promoter, an Albumin promoter, a Prolamin promoter, a gliadin promoter, a Glutenin promoter, a Zein promoter, a α-kafirin promoter and a Hordein promoter. A lot of endosperm-specific promoters have been identified so far, and may be chosen from the group consisting of the Glutelin (GluB-1) from rice (Takaiwa et al., 1991, Plant Mol. Biol. 17: 875-885), α-Globulin (Glb-1) from rice (Nakase et al., 1996, Gene 170: 223-226), Albumin (RAG-1) from rice (Adachi et al., 1993, Plant Mol. Biol. 19: 239-248), Prolamin (NRP33) from rice (Sha et al., 1996, Biosc. Biotechnol. Biochem. 60: 335-337), Glutenin (Glu-1D-1) from wheat (Lamacchia et al., 2001, J. Exp. Botany 52: 243-250), Zein (Ze-19) from maize (Quattrocchio et al., 1990, Plant Mol. Biol. 15: 81-93), α-kafirin from sorghum (DeRose et al., 1996, Plant Mol. Biol. 32: 1029-1035) and Hordein (Hor2-4) from barley (Brandt, A. et al., 1985, Carlsberg Res. Commun. 50, 333-345).

The nucleic acids incorporated into the plant may be stably integrated into the genome of the plant cell. How to integrate said nucleic acids in the genome of the plant is well known by a skilled person in the art. The most commonly used method is based on Agrobacterium-mediated transformation which results in random insertion of the T-DNA fragment in the genome. However, in the method of the present invention there is no need to stably integrate said nucleic acids in the genome of the plants. Nevertheless, all plants or plant parts obtained by the method of the present invention will at least be engineered, transgenic or transformed for the plant GTPCHI.

As indicated above, the method of the present invention may be applied for all plants, in particular for a monocotyledonous plant or a dicotyledonous plant. Said monocotyledonous plant may be selected from the group consisting of rice, wheat, barley, oats, rye, sorghum, maize, sugarcane, pineapple, onion, bananas, coconut, lilies, turfgrasses, and millet. The group of the monocotyledonous plants comprises graminaecious crops. Monocots comprise one-quarter of all flowering plant species, most of these are orchids, grasses, sedges, palms, and aroids. Within the monocots, the Glumiflorae are the single-most important group of organisms on the planet today, providing us with corn, rice, wheat, and barley—the four highest grossing crops, as well as sugar cane. Their ecological importance in maintaining soil stability and providing turf has made them a common sight in yards and lawns around the world. The Glumiflorae comprise the grasses, sedges, rushes, and cattails. The dicotyledonous plant may selected from the group consisting of tomato, cucumber, squash, peas, beans, peanuts, spinach, broccoli, alfalfa, melon, chickpea, chicory, clover, kale, lentil, soybean, tobacco, potato, sweet potato, yams, cassaya, radish, cabbage, rape, apple and pear trees, citrus (including oranges, mandarins, grapefruit, lemons, and limes), grape, cotton, sunflower, strawberry, and lettuce.

In the method of the present invention, the plant may be a whole plant, a plant organ, a plant tissue, or a plant cell.

In addition, in the method of the present invention the plant may be a plant as defined above, but may also be a fungus or an alga. Indeed, it is known for a skilled person in the art that alga are closely related to plants. Therefore, the present invention indicates that any of the methods proposed to enrich plants with folate may also be applied to enrich alga with folate. This also applies for fungi.

Furthermore, in the method of the present invention, the folate may be tetrahydrofolate, 5-methyl tetrahydrofolate, folic acid, 5-formyltetrahydrofolate, 10-formylfolic acid, 5,10-methylenetetrahydrofolate, dihydrofolic acid, and/or 5,10-methenyltetrahydrofolate or combinations thereof; either in their poly- or in their monoglutamylated form.

When using the method of the present invention, the total folate content in the plant, measured by the analysis of the levels of tetrahydrofolate, 5-methyl tetrahydrofolate, folic acid, 5-formyltetrahydrofolate, 10-formylfolic acid, 5,10-methylenetetrahydrofolate, dihydrofolic acid, and 5,10-methenyltetrahydrofolate; either in their poly- or in their monoglutamylated form, may be higher than the total folate content present in the wild type plant. For instance, said total folate content may be 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0, 50.0, 51.0, 52.0, 53.0, 54.0, 55.0, 56.0, 57.0, 58.0, 59.0, 60.0, 61.0, 62.0, 63.0, 64.0, 65.0, 66.0, 67.0, 68.0, 69.0, 70.0, 71.0, 72.0, 73.0, 74.0, 75.0, 76.0, 77.0, 78.0, 79.0, 80.0, 81.0, 82.0, 83.0, 84.0, 85.0, 86.0, 87.0, 88.0, 89.0, 90.0, 91.0, 92.0, 93.0, 94.0, 95.0, 96.0, 97.0, 98.0, 99.0, 100.0, 101.0, 102.0, 103.0, 104.0, 105.0, 106.0, 107.0, 108.0, 109.0, 110.0, 111.0, 112.0, 113.0, 114.0, 115.0, 116.0, 117.0, 118.0, 119.0, 120.0, 121.0, 122.0, 123.0, 124.0, 125.0, 126.0, 127.0, 128.0, 129.0 or 130.0 nmol (and possibly even higher) per gram fresh weight. In the examples, for the transgene rice seed a total folate content between 6-38.3 (=270-1723 μg per 100 gram fresh weight) nmol per gram fresh seed weight has been observed. However, it is known for a skilled person that the folate content may vary depending on the transgenic plant analyzed. Consequently when analyzing more plants, lower or higher folate content may be observed. Therefore, the present application indicates that the folate content observed in the transgenic plants may be defined as being higher compared to the folate content present in wild type plants. For instance, for rice seed, the increased folate content should be seen as higher than 0.4 nmol per gram fresh seed weight. Furthermore, the values mentioned above should not be seen as limiting to the seeds of the plant but represents the value for the total folate content which may be obtained when the method of the invention is followed for any cell of the plant, wherein the folate levels after genetic modification using the method of the present invention are higher than the wild type levels in the same plant part. As indicated in Table 1, the wild type levels of folates in different plants differ; the obtained folate level should thus be compared to the wild type folate level of the same plant type. In the plant of the examples the genes used to increase the folate content are only expressed in the seed of the plant, therefore only the folate content in the seed has been analyzed. Nevertheless, when applying the concept of the present invention for other parts of the plant (i.e. root), thereby using for instance promoters specific for said plant part (i.e. root specific promoter), the same effect will be found. The final folate level reached also depends on the strength of the promoters used.

The 0.4 nmol value found for the wild type rice seed, was obtained using the methods described in the present application (see Table 2). This stands somewhat in contrast with the folate value published by the USDA National Nutrient Database for wild type rice seed (0.13-0.18 nmol) indicated in Table 1. The latter values were obtained using methods other than the methods used in the present application. However, said values may still be used to compare values determined by the same method. Therefore, values referred to in the present invention in relation to the methods or the plants of the present invention should thus be seen as determined by the methods disclosed in the present application.

When following the method of the present invention, plants may be obtained wherein the molar ratio of folates/PABA/pterins is 1/<2.5/<0.75. In the examples of the present application, it is indicated that for the ‘seeds’ of the plants analyzed the ratio is 1/0.5/0.013. The 1/<2.5/<0.75 ratio should not be seen as limiting to the seeds of the plant but represents the ratio which may be obtained for any cell of the plant when the method of the invention is followed. In the plants of the examples the genes used to increase the folate content are only expressed in the seed of the plant, therefore only the folate content in the seed has been analyzed. In addition, as indicated above for folate, when analyzing more plants, lower or higher PABA and pterins contents in the plant may be observed. Consequently, the general ratio proposed in the present application, obtained in the plant of the present invention is 1/<2.5/<0.75, as the ratio found by Hanson in tomato is 1/2.5/0.75. According to the present invention, the value for PABA in said ratio may be 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, 0.09, 0.08, 0.07, 0.06, 0.05, 0.04, 0.03, 0.02, or 0.01; the value for pterins in said ratio may be 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50, 0.49, 0.48, 0.47, 0.46, 0.45, 0.44, 0.43, 0.42, 0.41, 0.40, 0.39, 0.38, 0.37, 0.36, 0.35, 0.34, 0.33, 0.32, 0.31, 0.30, 0.29, 0.28, 0.27, 0.26, 0.25, 0.24, 0.23, 0.22, 0.21, 0.20, 0.19, 0.18, 0.17, 0.16, 0.15, 0.14, 0.13, 0.12, 0.11, 0.10, 0.09, 0.08, 0.07, 0.06, 0.05, 0.04, 0.039, 0.038, 0.037, 0.036, 0.035, 0.034, 0.033, 0.032, 0.031, 0.030, 0.029, 0.028, 0.027, 0.026, 0.025, 0.024, 0.023, 0.022, 0.021, 0.020, 0.019, 0.018, 0.017, 0.016, 0.015, 0.014, 0.013, 0.012, 0.011, 0.010, 0.009, 0.008, 0.007, 0.006, 0.005, 0.004, 0.003, 0.002, or 0.001.

In the method of the present invention, at least one polynucleotide to enrich the plant for vitamins (such as vitamin A, B, E or C), for microelements (such as iron, zinc, or magnesium), and/or for medicinal components (said components being components which are used to fight diseases or disorders such as anemia or immune deficiency or artherosclerosis or dementia or cancer) may be further incorporated. In reducing the risk for artherosclerosis, polyphenolic compounds may be used, while in the control of dementia, plant cholinesterase inhibitors may be included. Alternatively, the transgenic plant, plant organ, plant tissue, or plant cell obtainable or obtained by a method of the present invention may be mixed with said vitamins, microelements and/or medicinal compounds.

The present invention further contemplates a transgenic plant, plant organ, plant tissue, or plant cell obtainable or obtained by a method of the present invention. The present invention also relates to a harvestable part or a propagule of said plant. Said harvestable part may be selected from the group consisting of seeds, leaves, fruits, stem cultures, rhizomes, tubers and bulbs. The present invention also contemplates progeny, plant organ, plant tissue, plant cell, harvestable part, propagule, seed, leaf, fruit, stem culture, rhizome, tuber and/or bulb derived from the plant of the invention.

The present invention further relates to plant breeding material comprising a plant, plant organ, plant tissue, plant cell, harvestable part, propagule, seed or progeny of the present invention. For instance, the present invention relates to plant breeding material comprising a plant, plant organ, plant tissue, plant cell, harvestable part, propagule, seed or progeny thereof, overexpressing plant GTPCHI. Said plant breeding material may be used to cross with plants overexpressing one or more enzymes which form part of the folate synthesis pathway.

More specifically, the present invention may be seen as providing a transgenic plant overexpressing a polynucleotide encoding a plant GTP cyclohydrolase I (GTPCHI) enzyme. In said transgenic plant said plant GTP cyclohydrolase I (GTPCHI) enzyme may for instance be derived from Arabidopsis thaliana represented by SEQ ID NO:1 or SEQ ID NO:67. Said transgenic plant may further comprise a polynucleotide encoding a plant 4-amino-4-deoxychorismate synthase (ADCS). The polynucleotide encoding the 4-amino-4-deoxychorismate synthase may be derived from Arabidopsis thaliana represented by SEQ ID NO:11. According to the present invention, the transgenic plant produced may be a rice plant. In said transgenic plant the folate level is increased compared to the folate level present in a wild type plant. The folate increase may be performed in the endosperm of the seed of said plant. In addition, a product comprising the transgenic plant of the present invention, or a seed obtained from the plant of the present invention can be made. However, these are only examples of possible embodiments of the invention.

The present invention may also be seen as relating to a monocot plant with increased folate level, wherein said plant may be a rice plant. As indicated above, said plant may have a folate level which is increased compared to the folate level present in a wild type plant. Said monocot plant may overexpress a plant polynucleotide encoding the GTP cyclohydrolase I (GTPCHI) enzyme. More specifically, the polynucleotide encoding the GTP cyclohydrolase I (GTPCHI) enzyme may be derived from Arabidopsis thaliana represented by SEQ ID NO:1 or SEQ ID NO:67. In addition, said monocot plant may further comprise a plant polynucleotide encoding a 4-amino-4-deoxychorismate synthase (ADCS); wherein the polynucleotide encoding the 4-amino-4-deoxychorismate synthase may be derived from Arabidopsis thaliana represented by SEQ ID NO:11. For said monocot plant, the folate increase may be performed in the endosperm of the seed of said plant. A product comprising the monocot plant of the present invention, and, a seed obtained from said monocot plant are also envisaged by the present invention. However, these are only examples of possible embodiments of the invention.

The present invention may also be seen as providing a seed of a plant with increased folate level, wherein the seed may be a rice kernel. The folate increase in said seed may be performed in the endosperm of the seed. Folate levels may be higher than 0.4 nmol per gram fresh seed weight, possibly varying between 6-38.3 nmol per gram fresh seed weight. In said seed, the level of 5-methyl tetrahydrofolate, which is part of the total folate content of the seed, may be higher than the 5-methyl tetrahydrofolate present in the seed of a wild type plant. In the wild type plant the level of 5-methyl tetrahydrofolate is 0.27 nmol per gram fresh seed weight; the level of 5-methyl tetrahydrofolate in the seed of the plant of the present invention may thus be defined as being higher than 0.27 nmol per gram fresh seed weight. As indicated above, said value should not be seen as limiting to the seeds of the plant but represents the value for 5-methyl tetrahydrofolate which may be obtained when for any cell of the plant the method of the invention is followed. In the plant of the present invention the genes used to increase the folate content are only expressed in the seed of the plant, therefore only the folate content in the seed has been analyzed. In addition, as indicated above, when analyzing more plants, lower or higher 5-methyl tetrahydrofolate content may be observed. Therefore, the present application indicates that the 5-methyl tetrahydrofolate content observed in the transgenic plants may be defined as being higher compared to the 5-methyl tetrahydrofolate content present in wild type plant. In the examples, for the transgenic rice seed a 5-methyl tetrahydrofolate content between 0.27-36.8 nmol per gram fresh seed weight has been observed. However, it is known for a skilled person that the 5-methyl tetrahydrofolate content may vary depending on the transgenic plant analyzed. According to the invention, said 5-methyl tetrahydrofolate content may be 0.27, 0.28, 0.29, 0.30, 0.31, 0.32, 0.33, 0.34, 0.35, 0.36, 0.37, 0.38, 0.39, 0.40, 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0, 50.0, 51.0, 52.0, 53.0, 54.0, 55.0, 56.0, 57.0, 58.0, 59.0, 60.0, 61.0, 62.0, 63.0, 64.0, 65.0, 66.0, 67.0, 68.0, 69.0, 70.0, 71.0, 72.0, 73.0, 74.0, 75.0, 76.0, 77.0, 78.0, 79.0, 80.0, 81.0, 82.0, 83.0, 84.0, 85.0, 86.0, 87.0, 88.0, 89.0, 90.0, 91.0, 92.0, 93.0, 94.0, 95.0, 96.0, 97.0, 98.0, 99.0, 100.0, 101.0, 102.0, 103.0, 104.0, 105.0, 106.0, 107.0, 108.0, 109.0, 110.0, 111.0, 112.0, 113.0, 114.0, 115.0, 116.0, 117.0, 118.0, 119.0, 120.0, 121.0, 122.0, 123.0, 124.0, 125.0, 126.0, 127.0, 128.0, 129.0, or 130.0 nmol (and possibly even higher) per gram fresh seed weight. As indicated above the seed may comprise a polynucleotide encoding a plant GTP cyclohydrolase I (GTPCHI) enzyme; wherein the polynucleotide encoding the GTP cyclohydrolase I (GTPCHI) enzyme may be derived from Arabidopsis thaliana represented by SEQ ID NO:1 or SEQ ID NO:67. Said seed may further comprise a polynucleotide encoding a plant 4-amino-4-deoxychorismate synthase (ADCS); wherein the polynucleotide encoding the 4-amino-4-deoxychorismate synthase may be derived from Arabidopsis thaliana represented by SEQ ID NO:11. The present invention also encompasses a product comprising the seed of the present invention. However, these are only examples of possible embodiments of the invention.

Another embodiment of the invention is an isolated polynucleotide comprising a polynucleotide sequence encoding an enzyme derived from a plant that catalyzes the synthesis of dihydroneopterin-PPP from GTP, and, a polynucleotide sequence encoding one or more enzymes that catalyze synthesis of PABA. Said isolated polynucleotide may for instance comprise a polynucleotide sequence encoding a plant GTP cyclohydrolase I (GTPCHI) enzyme comprising the amino acid sequence chosen from the group consisting of a sequence represented by SEQ ID NO: 2, 4, 6, 8, 10, 68 and 70, an analogue, a homologue, and, an enzymatically functional fragment of any of SEQ ID NO: 2, 4, 6, 8, 10, 68 or 70, wherein said analogue or homologue is at least 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75% 76%, 77%, 78%, 79%, 80%, 81%, 82%, 83%, 84%, 85%, 86%, 87%, 88%, 89%, 90%, 91%, 92%, 93%, 94%, 95% 96%, 97%, 98% or at least 99% identical to the amino acid sequence of any of SEQ ID NO: 2, 4, 6, 8, 10, 68 or 70; and a polynucleotide sequence encoding a plant 4-amino-4-deoxy-chorismate synthase (ADCS) enzyme comprising the amino acid sequence chosen from the group consisting of a sequence represented by SEQ ID NO:12, 14 and 16, an analogue, a homologue, and an enzymatically functional fragment of any of SEQ ID NO:12, 14, or 16, wherein said analogue or homologue is at least 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% identical to the amino acid sequence of any of SEQ ID NO:12, 14, or 16; and wherein said enzymatically functional fragment is chosen from the group consisting of the putative chloroplastic transit peptide domain (1-87 aa of the polypeptide represented by SEQ ID NO:12 or corresponding domains in SEQ ID NO:14 or 16), the putative Pab A domain (88-334 aa of the polypeptide represented by SEQ ID NO:12 or corresponding domains in SEQ ID NO:14 or 16), and the putative Pab B domain (430-919 aa of the polypeptide represented by SEQ ID NO:12 or corresponding domains in SEQ ID NO:14 or 16). More specifically, said polynucleotide may comprise the polynucleotide sequence chosen from the group consisting of a sequence represented by SEQ ID NO:1, 3, 5, 7, 9, 67, 69 and 71 and the polynucleotide sequence chosen from the group consisting of a sequence represented by SEQ ID NO:11, 13 and 15, or a polynucleotide sequence that hybridizes under stringent conditions with a polynucleotide sequence complementary to the sequence chosen from the group consisting of a sequence represented by SEQ ID NO:1, 3, 5, 7, 9, 67, 69 and 71 and a polynucleotide sequence that hybridizes under stringent conditions with a polynucleotide sequence chosen from the group consisting of a sequence represented by SEQ ID NO:11, 13 and 15. Also other combinations of said polynucleotides are possible. According to the invention, said isolated polynucleotide may be used to increase the folate content of a plant. The present invention further contemplates an expression vector comprising the isolated polynucleotide of the present invention and regulatory sequences operatively linked to said polynucleotides such that the polynucleotides are expressed in the plant cell in order to increase the level of folate in said plant. When applying the concept of the present invention for rice, said regulatory sequences may for instance comprise a rice endosperm promoter represented by SEQ ID NO:23 (Glob promoter) and/or a rice endosperm promoter represented by SEQ ID NO:24 (GluB1 promoter). The present invention relates then to a transgenic rice cell transformed with the expression vector of the present invention, and to a transgenic rice plant grown from said transgenic rice cell. Said transgenic rice plant may belong to different genera, for instance, said rice plant may belong to the genus Oryza or to the genus Zinania. Examples of possible rice species may be Oryza sativa and Oryza glaberrima including the accession Nipponbare japonica, Zizania palustris, Zizania texana and Zizania latifolia. However, as indicated before, the method of the present invention may be applied for different plant (sub)species and is not limited to the (sub)species mentioned in the present application. Seeds of the transgenic rice plant can be obtained. In said seed, increased folate levels may be detected. The total level of folate in the seed of a plant (endosperm) may be between 6-38.3 nmol per gram fresh seed weight (=270-1723 μg per 100 gr fresh weight). In the examples of the present application the folate content has been determined using a rice seed dried on the plant, mimicking natural harvest conditions. However, the folate content present in the seed stored for a long time may deviate (showing a decrease in content) from the folate content present in the seed isolated from the plant and stored only for a short period. The above only provides examples of possible embodiments of the invention.

The nucleic acid sequences according to the invention as defined above may, advantageously, be included in a suitable expression vector which may be introduced, transformed, transfected or infected into a host cell. In such an expression vector the nucleic acid is operably linked to a promoter, control and terminator sequences, or the like, to ensure expression of the proteins according to the invention in a suitable host cell. The expression vector may also comprise a reporter molecule or a selectable marker. The expression vector may advantageously be a plasmid, cosmid, virus or other suitable vector which is known to those skilled in the art. The expression vector and the host cell defined herein also form part of the present invention.

The genetic construct may further comprise a selectable marker gene or genes that are functional in a cell into which said genetic construct is introduced. As used herein the term ‘selectable marker gene’ includes any gene which confers a phenotype on a cell in which it is expressed to facilitate the identification and/or selection of cells which are transfected or transformed with a genetic construct of the invention or a derivative thereof. Suitable selectable marker genes contemplated herein include the phosphinotricin resistance gene, neomycin phosphotransferase gene (nptII), hygromycin resistance gene, β-glucuronidase (GUS) gene, chloramphenicol acetyltransferase (CAT) gene and luciferase gene, amongst others.

The present application further indicates that all the plants, plant organs, plant tissues, plant cells, harvestable parts, propagules, seeds, progeny, products, plant breeding materials or cells of the present invention may be used for many different applications. For instance, these may be used to cross breed with other plants. In addition, said plants, plant organs, plant tissues, plant cells, harvestable parts, propagules, seeds, progeny, products, plant breeding materials or cells may also be used as a medicament or as biofortification to enrich the quality of the food intake of an animal or human subject. In particular, said plants, plant organs, plant tissues, plant cells, harvestable parts, propagules, seeds, progeny, products, plant breeding materials or cells may be used for the manufacture of a medicament to prevent folate insufficiency, or, for the treatment of folate insufficiency or diseases or disorders resulting from said insufficiency in an animal or human subject. Said disease may be a neural tube defect (spina bifida, anencephaly, and other defects to the neural tube), megaloblastic anemia, neurodegenerative diseases (such as Alzheimer's disease), cancer or a vascular disease. It is generally accepted that folate may prevent said diseases or disorders. Furthermore, it is known that folate may be used to prevent and treat anemia. According to the present invention, the plant, plant organ, plant tissue, plant cell, harvestable part, propagule, seed, progeny, product, plant breeding material, or, cell of the present invention, or a derivative thereof, may be a medicated foodstuff and administered orally under dried or fresh form. Said derivative may comprise a liquid tonic and said tonic is administered orally. As the conditions and minimum doses for administration of folate supplements to treat patients with is well known, the treatment of patients using the products of the present invention may easily be derived and applied. The present invention also relates thus to a food additive comprising the plant, plant organ, plant tissue, plant cell, harvestable part, propagule, seed, progeny, product, plant breeding material, or, cell of the present invention, or a derivative thereof.

The present invention also contemplates a pharmacological composition comprising a plant, plant organ, plant tissue, plant cell, harvestable part, propagule, seed, progeny, product, plant breeding material or cell of the present invention, or a pharmacologically acceptable derivative thereof and optionally a pharmaceutical acceptable carrier, diluent or excipient. Sterilisation may be carried out in several ways, for example, by incorporating sterilising agents in the composition or by irradiation. They may also be prepared in the form of sterile solid compositions which may be dissolved at the time of use in sterile water or any other sterile medium. The present invention can also comprise adjuvants which are well known to a person skilled in the art (vitamin C, antioxidant agents etc.) capable of being used in synergy with the products according to the invention in order to improve the treatments of the diseases or disorders to be treated or prevented. The compositions are preferentially prepared as solid forms; liquid solutions or suspensions may also be prepared. The products of present invention may be mixed with diluents or excipients which are compatible and physiologically tolerable. Suitable diluents and excipients are for example, water, saline, dextrose, glycerol, food components or the like, and combinations thereof. In addition, if desired the compositions may contain minor amounts of auxiliary substances such as wetting or emulsifying, stabilizing or pH buffering agents. Compositions can be provided together with physiologically tolerable liquid, gel or solid carriers, diluents, adjuvants and excipients. Formulations may contain such normally employed additives as binders, fillers, carriers, preservatives, stabilizing agents, emulsifiers, buffers and excipients as for example, pharmaceutical grades of mannitol, lactose, starch, magnesium stearate, sodium saccharin, cellulose, magnesium carbonate and the like.

The pharmaceutical forms must also be stable under the storage conditions and must be preserved against the contaminating action of microorganisms such as bacteria and fungi. The carrier can be solvent or dispersion medium containing, for example water, ethanol, polyol (for example glycerol, propylene glycol, and liquid polyethylene glycol and the like), suitable mixtures thereof, and vegetable oils. The prevention of the action of microorganisms can be brought about by various antibacterial and antifungal agents, for example, parabens, chorobutanol, phenol, sorbic acid, thiomersal and the like. Carriers and/or diluents suitable for veterinary use include any and all solvents, dispersion media, aqueous solutions, coatings, antibacterial and antifungal agents, isotonic and absorption delaying agents and the like. The use of such media and agents for pharmaceutically active substances is well known in the art. Except insofar as any conventional media or agent is incompatible with the active reagent, use thereof in the composition is contemplated. Supplementary active reagents can also be incorporated into the compositions.

According to the present invention, products containing a plant, plant organ, plant tissue, plant cell, harvestable part, propagule, seed, progeny, product, plant breeding material or cell of the present invention, or a pharmacologically acceptable derivative thereof, may also be part of a combined preparation for simultaneous, separate or sequentially use in folate deficiency therapy.

Plant cells, differentiated or undifferentiated tissues from a plant with enhanced expression of folate biosynthesis genes which are not present in animals (such as those leading to pABA formation), may also be used to screen for novel compounds with herbicidal activity. The advantage of using a cell line, plant, or plant tissue which contains a very high amount of folates as obtained by the strategy in the present invention, resides in the fact that only those compounds with very strong activity will be selected, which may hasten the discovery of useful derivates.

On the other hand, antifolate antimalarial drugs such as inhibitors of DHFR (e.g. methotrexate) and DHPS (e.g. sulfonamides) can be screened for in an automated manner if the transgene plant cells with a high folate content, resulting from overexpression of folate biosynthesis genes, are used.

As indicated above, the present invention relates to a transgenic plant with a folate content higher than the folate content present in the wild type plant and overexpressing a plant GTP cyclohydrolase I (GTPCHI) enzyme; wherein said plant GTPCHI enzyme comprises the amino acid sequence chosen from the group consisting of a sequence represented by SEQ ID NO:2, 4, 6, 8, 10, 68 and 70, an analogue, a homologue, and, an enzymatically functional fragment of any of SEQ ID NO:2, 4, 6, 8, 10, 68 or 70, wherein said analogue or homologue is at least 50% identical to the amino acid sequence represented by any of SEQ ID NO:2, 4, 6, 8, 10, 68 or 70. Said transgenic plant may further overexpress a plant 4-amino-4-deoxychorismate synthase (ADCS), wherein said ADCS enzyme comprises the amino acid sequence chosen from the group consisting of a sequence represented by SEQ ID NO:12, 14 and 16, an analogue, a homologue, and, an enzymatically functional fragment of any of SEQ ID NO:12, 14 or 16, wherein said analogue or homologue is at least 50% identical to the amino acid sequence represented by any of SEQ ID NO:12, 14 or 16; wherein said enzymatically functional fragment is chosen from the group consisting of the putative chloroplastic transit peptide domain (1-87 aa of the polypeptide represented by SEQ ID NO:12 or corresponding domains in SEQ ID NO:14 or 16), the putative Pab A domain (88-334 aa of the polypeptide represented by SEQ ID NO:12 or corresponding domains in SEQ ID NO:14 or 16), and the putative Pab B domain (430-919 aa of the polypeptide represented by SEQ ID NO:12 or corresponding domains in SEQ ID NO:14 or 16).

The present invention also relates to a plant organ, plant tissue, plant cell, harvestable part, propagule, seed, progeny or plant breeding material with an increased folate content compared to the wild type obtained from a plant described above and overexpressing a plant GTPCHI enzyme comprising the amino acid sequence chosen from the group consisting of a sequence represented by SEQ ID NO:2, 4, 6, 8, 10, 68 and 70, an analogue, a homologue, and, an enzymatically functional fragment of any of SEQ ID NO:2, 4, 6, 8, 10, 68 or 70, wherein said analogue or homologue is at least 50% identical to the amino acid sequence represented by any of SEQ ID NO:2, 4, 6, 8, 10, 68 or 70.

The present invention further relates to a method for increasing folate levels in a plant comprising (a step introducing) the overexpression of a plant GTPCHI enzyme comprising the amino acid sequence chosen from the group consisting of a sequence represented by SEQ ID NO: 2, 4, 6, 8, 10, 68 and 70, an analogue, a homologue, and, an enzymatically functional fragment of any of SEQ ID NO:2, 4, 6, 8, 10, 68 or 70, wherein said analogue or homologue is at least 50% identical to the amino acid sequence represented by any of SEQ ID NO:2, 4, 6, 8, 10, 68 or 70. Said method may further comprise (a step introducing) the overexpression of an ADCS enzyme comprising the amino acid sequence chosen from the group consisting of a sequence represented by SEQ ID NO:12, 14 and 16, an analogue, a homologue, and, an enzymatically functional fragment of any of SEQ ID NO:12, 14 or 16, wherein said analogue or homologue is at least 50% identical to the amino acid sequence represented by any of SEQ ID NO:12, 14 or 16; wherein said enzymatically functional fragment is chosen from the group consisting of the putative chloroplastic transit peptide domain (1-87 aa of the polypeptide represented by SEQ ID NO:12 or corresponding domains in SEQ ID NO:14 or 16), the putative Pab A domain (88-334 aa of the polypeptide represented by SEQ ID NO:12 or corresponding domains in SEQ ID NO:14 or 16), and the putative Pab B domain (430-919 aa of the polypeptide represented by SEQ ID NO:12 or corresponding domains in SEQ ID NO:14 or 16).

The present invention further relates to an isolated polynucleotide comprising a polynucleotide sequence encoding a plant GTP cyclohydrolase I (GTPCHI) enzyme comprising the amino acid sequence chosen from the group consisting of a sequence represented by SEQ ID NO:2, 4, 6, 8, 10, 68 and 70, an analogue, a homologue, and, an enzymatically functional fragment of any of SEQ ID NO:2, 4, 6, 8, 10, 68 or 70, wherein said analogue or homologue is at least 50% identical to the amino acid sequence of any of SEQ ID NO:2, 4, 6, 8, 10, 68 or 70; and a polynucleotide sequence a plant 4-amino-4-deoxychorismate synthase (ADCS) enzyme comprising the amino acid sequence chosen from the group consisting of a sequence represented by SEQ ID NO:12, 14 and 16, an analogue, a homologue, and, an enzymatically functional fragment of any of SEQ ID NO:12, 14 or 16, wherein said analogue or homologue is at least 50% identical to the amino acid sequence of any of SEQ ID NO:12, 14 or 16; and wherein said enzymatically functional fragment is chosen from the group consisting of the putative chloroplastic transit peptide domain (1-87 aa of the polypeptide represented by SEQ ID NO:12 or corresponding domains in SEQ ID NO:14 or 16), the putative Pab A domain (88-334 aa of the polypeptide represented by SEQ ID NO:12 or corresponding domains in SEQ ID NO:14 or 16), and the putative Pab B domain (430-919 aa of the polypeptide represented by SEQ ID NO:12 or corresponding domains in SEQ ID NO:14 or 16).

The present invention also relates to the use of an isolated polynucleotide described above to increase the folate content of a plant. The present invention also relates to an expression vector comprising said polynucleotide and regulatory sequences operatively linked to said polynucleotides such that the polynucleotides are expressed in the plant cell in order to increase the level of folate in said plant.

The present invention further relates to a transgenic plant cell transformed with the above mentioned expression vector. The present invention also relates to a transgenic plant, plant organ, plant tissue, harvestable part, propagule seed, progeny or plant breeding material with an increased folate content derived from said transgenic cell, or, a transgenic plant obtained by a method described above.

The present invention also relates to a product, a food additive or a pharmacological composition comprising a transgenic plant, plant organ, plant tissue, plant cell, harvestable part, propagule, seed, progeny or plant breeding material described above.

The present invention also relates to the use of a plant, plant organ, plant tissue, plant cell, harvestable part, propagule, seed, progeny, plant breeding material, product, additive or composition described above to cross breed with other plants, for use as a medicament, as biofortification to enrich the quality of the food intake of an animal or human subject, or, for the manufacture of a medicament to prevent folate insufficiency, or, for the treatment of folate insufficiency or diseases or disorders resulting from said insufficiency in an animal or human subject.

### BRIEF DESCRIPTION OF THE FIGURES

FIG. 1. Folate structure and biosynthesis. A. Chemical structure of folates. B. Simplified scheme of folate biosynthesis in plants. Engineered steps and enzymes are bolded. GTPCHI, GTP—cyclohydrolase I; HPPK, hydroxymethyldihydropterin pyrophosphokinase; DHPS, dihydropteroate synthase; DHFS, dihydrofolate synthase; DHFR, dihydrofolate reductase; FPGS, folylpolyglutamate synthase; ADCS, aminodeoxychorismate synthase; ADCL, aminodeoxychorismate lyase; DHN-P-P-P, dihydroneopterin triphosphate; DHN, dihydroneopterin; ADC, aminodeoxychorismate; p-ABA—para-aminobenzoic acid.

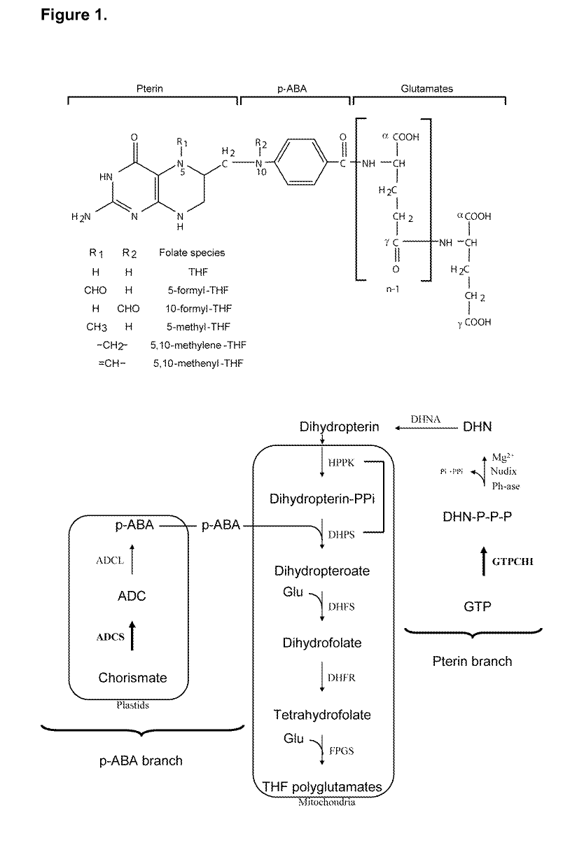

FIG. 2. Schematic representation of the T-DNA in plant transformation vectors. Open pointers depict promoters, filled pointers depict cDNA coding regions, grey bars indicate transcriptional terminators. LB and RB, left and right T-DNA borders, respectively; T35S and Tn, transcriptional terminators of the 35S transcript of cauliflower mosaic virus and nopaline synthase gene of Agrobacterium tumefaciens, respectively; 35S, Glob and GluB1, core cauliflower mosaic virus 35S promoter with duplicated enhancer sequence, rice globulin and rice glutelin B1 promoters, respectively; GTPCHI, coding sequence of Arabidopsis GTP cyclohydrolase I; ADCS, coding sequence of ADC synthase from Arabidopsis; hptII, hygromycin phosphotransferase gene (hygromycin selectable marker).

FIG. 3. Southern blotting-hybridization of genomic DNA samples isolated from leaves of T1 individuals obtained by self-crossing of primary (T0) GA lines for which single copy transformation events were determined by Q-PCR. Genomic DNA was cut with EcoRV restriction endonuclease, resolved by agarose electrophoresis, blotted onto a membrane and hybridized with radioactively labeled probe. A. Schematic representation of GA construct T-DNA. The probe is indicated by an open horizontal bar. B. P-imager (Storm 860, Amersham Biosciences) scan of the membrane after the hybridization. Samples of different individuals of the T1 progeny of the same T0 transgenic lines are grouped and indicated by horizontal bars. A single band obtained for all individuals of the progeny of a transgenic line confirms a single T-DNA integration event.

FIG. 4. Expression analysis of introduced GTP cyclohydrolase I (GTPCHI) and ADC synthase (ADCS) genes in transgenic rice lines by northern hybridization with radioactive probes. Samples from seeds of homozygous T2 and T3 plants are underlined with regular and bold lines, respectively, while the dashed line depicts a hemizygous plant. V, control transformed with the “empty” vector. Hybridization with a 25S rDNA probe was used as the loading control.

FIG. 5. Expression analysis of the genes introduced in seeds of transgenic rice plants. Total RNA was isolated from mature green seeds, resolved in a denaturing agarose gel, transferred to a membrane and hybridized with the corresponding radioactive probe. Hybridization with 25S rDNA was used as a loading control. Seeds of homozygous T2 and T3 plants are underlined with regular and bold lines, respectively. A dashed line indicates a sample from a hemizygous T2 individual. V corresponds to seeds from a control plant transformed with the empty vector. A, samples from A-lines, ADCS probe; B, samples from GA lines ADCS probe; C, samples from G and GA lines, GTPCHI probe.

FIG. 6. Analysis of p-ABA, pteridines, and folate content in seeds of transgenic rice plants transformed with A, G and GA constructs. Values are means of 2 independent seed samples, error bars are standard deviations. Sample annotations are as in FIG. 3. V, control transformed with the “empty” vector. A. Total p-ABA and folate levels in seeds of A-lines. B. Total pteridines and folate levels in G-lines. C. Total p-ABA, pteridines, and folate levels in GA-lines. D. Representation of the main folate species in seeds of GA lines as compared to wild type seeds calculated as percentages of total folate for each line. Values are averages of 5 GA transgenic lines or 3 independent measurements in case of wild type seeds. Error bars are standard deviations. H₄PteGlu—tetrahydrofolate; 5-CH₃—H₄PteGlu—5-methyltetrahydro folate; PteGlu—folic acid; 5-CHO—H₄PteGlu—5-formyltetrahydrofolate; 10-CHO-PteGlu—10-formylfolic acid; 5,10=CH—H₄PteGlu—5,10-methenyltetrahydrofolate.

FIG. 7. Southern blotting hybridization of primary transformed rice plants (T0) with a GADL construct. The genomic DNA was cut with EcoRV restriction enzyme. A fragment of ADCS coding sequence was used as a probe.

FIG. 8. Expression analysis of the introduced genes in seeds of F1 rice plants, transformed with GADL constructs. A, B, C, hybridizations with GTPCHI, ADCS, and ADCL probes, respectively. 1, rice line expressing GTPCHI, 2-8, GADL transformed plants, 9, plant transformed with empty vector.

FIG. 9. Plasmid map of the plant transformation vector pMOD35hA carrying the folate gene ADCS. Sm/SpR Streptomycin/Spectinomycin resistance gene; hptII (Hyg R) hygromycin phosphotransferase gene (hygromycin resistance); ADCS Full-length cDNA of ADC synthase (At2g28880) from Arabidopsis; STA from pVS1 STA region from pVS1 plasmid; bom site pBR322 bom site from pBR322; LB left border repeat from C58 T-DNA; attB1 Gateway recombination repeat; attB2 Gateway recombination repeat; RB right border T-DNA repeat; CaMV35S2 CaMV 35S promoter duplicated; GluB1 Promoter and 5′ UTR region of the GluB1 seed storage protein from Oryza sativa (japonica), corresponds to the 1130-2335 by of the AY427569 accession; pVS1-REP replication origin of pVS1; pBR322 origin pBR322; T35S transcriptional terminator of CaMV 35S; Tnos transcription terminator from NOS gene A. tumefaciens.

FIG. 10. Plasmid map of the plant transformation vector pMOD35hG carrying the folate gene GTPCHI. Sm/SpR Streptomycin/Spectinomycin resistance gene; hptII (Hyg R) hygromycin phosphotransferase gene (hygromycin resistance); GTPCHI Full-length cDNA for GTP cyclohydrolase I (At3g07270) from Arabidopsis; RB right border T-DNA repeat; STA from pSV1 STA region from pVS1 plasmid; bom site pBR322 bom site from pBR322; LB left border repeat from C58 T-DNA; attB1 Gateway recombination repeat; attB2 Gateway recombination repeat; CaMV35S2 CaMV 35S promoter duplicated; pGlob Promoter and 5′ UTR of the seed storage 26 kDa alpha globulin from Oryza sativa (japonica), corresponds to 58-981 by of AY427575.1; pVS1-REP replication origin of pVS1; pBR322 ori origin pBR322; T35S transcriptional terminator of CaMV 35S; Tnos transcription terminator from NOS gene A. tumefaciens.

FIG. 11. Plasmid map of the plant transformation vector pMOD35hGA carrying the folate genes GTPCHI and ADCS. Sm/SpR Streptomycin/Spectinomycin resistance gene; hptII (Hyg R) hygromycin phosphotransferase gene (hygromycin resistance); GTPCHI Full-length cDNA for GTP cyclohydrolase I (At3g07270) from Arabidopsis; ADCS Full-length cDNA of ADC synthase (At2g28880) form Arabidopsis; STA from pSV1 STA region from pVS1 plasmid; bom site pBR322 bom site from pBR322; LB left border repeat from C58 T-DNA; attB1 Gateway recombination repeat; attB2 Gateway recombination repeat; RB right border T-DNA repeat; CaMV35S2 CaMV 35S promoter duplicated; pGlob Promoter and 5′ UTR of the seed storage 26 kDa alpha globulin from Oryza sativa (japonica), corresponds to 58-981 by of AY427575.1; GluB1 Promoter and 5′ UTR region of the GluB1 seed storage protein from Oryza sativa (japonica), corresponds to the 1130-2335 by of the AY427569 accession; pVS1-REP replication origin of pVS1; pBR322 on origin pBR322; T35S transcriptional terminator of CaMV 35S; Tnos transcription terminator from NOS gene A. tumefaciens.

FIG. 12. Plasmid map of the plant transformation vector pMOD35hGAD carrying the folate genes GTPCHI, ADCS and DHNA. Sm/SpR Streptomycin/Spectinomycin resistance gene; hptII (Hyg R) hygromycin phosphotransferase gene (hygromycin resistance); DHNA2 Full-length cDNA for dihydroneopterin aldolase 2 (At5g62980) from Arabidopsis; GTPCHI Full-length cDNA for GTP cyclohydrolase I (At3g07270) from Arabidopsis; ADCS Full-length cDNA of ADC synthase (At2g28880) form Arabidopsis; STA from pSV1 STA region from pVS1 plasmid; bom site pBR322 bom site from pBR322; LB left border repeat from C58 T-DNA; attB1 Gateway recombination repeat; attB2 Gateway recombination repeat; RB right border T-DNA repeat; CaMV35S2 CaMV 35S promoter duplicated; BSV promoter from Banana streak virus (partial CDS ORFIII, accession AF215815); pGlob Promoter and 5′ UTR of the seed storage 26 kDa alpha globulin from Oryza sativa (japonica), corresponds to 58-981 by of AY427575.1; GluB1 Promoter and 5′ UTR region of the GluB1 seed storage protein from Oryza sativa (japonica), corresponds to the 1130-2335 by of the AY427569 accession; pVS1-REP replication origin of pVS1; pBR322 on origin pBR322; T35S transcriptional terminator of CaMV 35S; Tnos transcription terminator from NOS gene A. tumefaciens.

FIG. 13. Plasmid map of the plant transformation vector pMOD35hGADL1 carrying the folate genes GTPCHI, ADCS, DHNA and ADCL. Sm/SpR Streptomycin/Spectinomycin resistance gene; hptII (Hyg R) hygromycin phosphotransferase gene (hygromycin resistance); ADCL ADC lyase from Arabidopsis (At5g57850); DHNA2 Full-length cDNA for dihydroneopterin aldolase 2 (At5g62980) from Arabidopsis; GTPCHI Full-length cDNA for GTP cyclohydrolase I (At3g07270) from Arabidopsis; ADCS Full-length cDNA of ADC synthase (At2g28880) form Arabidopsis; STA from pSV1 STA region from pVS1 plasmid; bom site pBR322 bom site from pBR322; LB left border repeat from C58 T-DNA; attB1 Gateway recombination repeat; attB2 Gateway recombination repeat; RB right border T-DNA repeat; CaMV35S2 CaMV 35S promoter duplicated; BSV promoter from Banana streak virus (partial CDS ORFIII, accession AF215815); pGlob Promoter and 5′ UTR of the seed storage 26 kDa alpha globulin from Oryza sativa (japonica), corresponds to 58-981 by of AY427575.1; GluB1 Promoter and 5′ UTR region of the GluB1 seed storage protein from Oryza sativa (japonica), corresponds to the 1130-2335 by of the AY427569 accession; pVS1-REP replication origin of pVS1; pBR322 on origin pBR322; T35S transcriptional terminator of CaMV 35S; Tnos transcription terminator from NOS gene A. tumefaciens.

FIG. 14. Plasmid map of the plant transformation vector pMOD35hGADL2 carrying the folate genes GTPCHI, ADCS, DHNA and ADCL. Same as pMOD35hGADL1 with GluB1-ADCL-Tnos expression cassette in opposite orientation. Abbreviations as in FIG. 13.

FIG. 15. Plasmid map of the plant transformation vector pMOD35hGADF carrying the folate genes GTPCHI, ADCS and DHNA and the gene encoding hFBP. DHNA2 Full-length cDNA for dihydroneopterin aldolase 2 (At5g62980) from Arabidopsis; GTPCHI Full-length cDNA for GTP cyclohydrolase I (At3g07270) from Arabidopsis; ADCS Start: 6253 End: 9009 Full-length cDNA of ADC synthase (At2g28880) form Arabidopsis; Sm/SpR Streptomycin/Spectinomycin resistance gene; hptII (Hyg R) hygromycin phosphotransferase gene (hygromycin resistance); FBP Folate binding protein from human, cDNA fragment corresponds to 493-1159 of X62753.1; attB1 Gateway recombination repeat; attB2 Gateway recombination repeat; RB right border T-DNA repeat; STA from pSV1 STA region from pVS1 plasmid; bom site pBR322 bom site from pBR322; LB left border repeat from C58 T-DNA; BSV promoter from Banana streak virus (partial CDS ORFIII, accession AF215815); pGlob Promoter and 5′ UTR of the seed storage 26 kDa alpha globulin from Oryza sativa (japonica), corresponds to 58-981 by of AY427575.1; GluB1 Promoter and 5′ UTR region of the GluB1 seed storage protein from Oryza sativa (japonica), corresponds to the 1130-2335 by of the AY427569 accession; CaMV35S2 CaMV 35S promoter duplicated; pVS1-REP replication origin of pVS1; pBR322 on origin pBR322; T35S transcriptional terminator of CaMV 35S; Tnos transcription terminator from NOS gene A. tumefaciens.

FIG. 16. Plasmid map of the plant transformation vector pMOD35hFBP carrying the gene encoding hFBP. Sm/SpR Streptomycin/Spectinomycin resistance gene; hptII (Hyg R) hygromycin phosphotransferase gene (hygromycin resistance); FBP Folate binding protein from human, cDNA fragment corresponds to 493-1159 of X62753.1; STA from pSV1 STA region from pVS1 plasmid; born site pBR322 bom site from pBR322; LB left border repeat from C58 T-DNA; attB1 Gateway recombination repeat; attB2 Gateway recombination repeat; RB right border T-DNA repeat; CaMV35S2 CaMV 35S promoter duplicated; GluB1 Promoter and 5′ UTR region of the Oryza sativa seed storage protein GluB1, corresponds to the 1130-2335 by of AY427569 accession; pVS1-REP replication origin of pVS1; pBR322 ori origin pBR322; T35S transcriptional terminator of CaMV 35S; Tnos transcription terminator from NOS gene A. tumefaciens.

FIG. 17. Plasmid map of the plant transformation vector pMOD35h2xFBP carrying the gene encoding hFBP under two different promoters. Sm/SpR Streptomycin/Spectinomycin resistance gene; hptII (Hyg R) hygromycin phosphotransferase gene (hygromycin resistance); FBP Folate binding protein from human, cDNA fragment corresponds to 493-1159 of X62753.1; attB1 Gateway recombination repeat; attB2 Gateway recombination repeat; RB right border T-DNA repeat; STA from pSV1 STA region from pVS1 plasmid; bom site pBR322 bom site from pBR322; LB left border repeat from C58 T-DNA; GluB1 Promoter and 5′ UTR region of the Oryza sativa seed storage protein GluB1, corresponds to the 1130-2335 by of AY427569 accession; CaMV35S2 CaMV 35S promoter duplicated; pGlob Promoter of the seed storage 26 kDa alpha globulin from Oryza sativa (japonica), corresponds to 58-981 by of AY427575.1; pVS1-REP replication origin of pVS1; pBR322 on origin pBR322; T35S transcriptional terminator of CaMV 35S; Tnos transcription terminator from NOS gene A. tumefaciens.

FIG. 18. Plasmid map of the plant transformation vector pMODsgfpD carrying the folate gene DHFS. Sm/SpR Streptomycin/Spectinomycin resistance gene; sGFP Modified green fluorescent GFP; DHFS Full-length cDNA for dihydrofolate synthase (At5g41480) from A. thaliana; RB right border T-DNA repeat; STA from pSV1 STA region from pVS1 plasmid; bom site pBR322 bom site from pBR322; LB left border repeat from C58 T-DNA; attB1 Gateway recombination repeat; attB2 Gateway recombination repeat; 35S 35S promoter; GluB1 Promoter and 5′ UTR region of the Oryza sativa seed storage protein GluB1, corresponds to the 1130-2335 by of AY427569 accession; pVS1-REP replication origin of pVS1; pBR322 on origin pBR322; Tnos transcription terminator from NOS gene A. tumefaciens.

FIG. 19. Plasmid map of the plant transformation vector pMODsgfpDH carrying the folate genes DHFS and HPPK-DHPS. DHFS Full-length cDNA for dihydrofolate synthase (At5g41480) from A. thaliana; Sm/SpR Streptomycin/Spectinomycin resistance gene; sGFP Modified green fluorescent GFP; Pea HPPK_DHPS Start: 12903 End: 14450 Full-length cDNA of HPPK/DHPS from Pea (Y08611); RB right border T-DNA repeat; STA from pSV1 STA region from pVS1 plasmid; bom site pBR322 bom site from pBR322; LB left border repeat from C58 T-DNA; attB1 Gateway recombination repeat; attB2 Gateway recombination repeat; GluB1 Promoter and 5′ UTR region of the Oryza sativa seed storage protein GluB1, corresponds to the 1130-2335 by of AY427569 accession; 35S 35S promoter; pGlob Promoter of the seed storage 26 kDa alpha globulin from Oryza sativa (japonica), corresponds to 58-981 by of AY427575.1; pVS1-REP replication origin of pVS1; pBR322 ori origin pBR322; Tnos transcription terminator from NOS gene A. tumefaciens.

FIG. 20. Graphical representation of the alignment of the bacterial or mammalian GTPCHI or of the bacterial PabA and PabB with the corresponding plant enzymes. Plant GTPCHI contains 2 domains, with each domain being comparable to the bacterial or mammalian GTPCHI. Plant ADCS is a bi-functional enzyme representing activities of bacterial PabA and PabB enzymes as its N-terminal and C-terminal domains, respectively. Similar sequences are indicated in dark gray.

FIG. 21. Plasmid map of the plant transformation vector pMOD35hGAmtF carrying the folate genes for GTPCHI, ADCS, and mitochondrial FPGS. Sm/SpR Streptomycin/Spectinomycin resistance gene; hptII (Hyg R) hygromycin phosphotransferase gene (hygromycin resistance); GTPCHI Full-length cDNA for GTP cyclohydrolase I (At3g07270) from Arabidopsis; ADCS Full-length cDNA of ADC synthase (At2g28880) form Arabidopsis; mtFPGS Full-length cDNA for mitochondrial FPGS (AJ271786) from Arabidopsis; STA from pSV1 STA region from pVS1 plasmid; bom site pBR322 bom site from pBR322; LB left border repeat from C58 T-DNA; attB1 Gateway recombination repeat; attB2 Gateway recombination repeat; RB right border T-DNA repeat; CaMV35S2 CaMV 35S promoter duplicated; pGlob Promoter and 5′ UTR of the seed storage 26 kDa alpha globulin from Oryza sativa (japonica), corresponds to 58-981 by of AY427575.1; GluB1 Promoter and 5′ UTR region of the GluB1 seed storage protein from Oryza sativa (japonica), corresponds to the 1130-2335 by of the AY427569 accession; pVS1-REP replication origin of pVS1; pBR322 ori origin pBR322; T35S transcriptional terminator of CaMV 35S; Tnos transcription terminator from NOS gene A. tumefaciens.

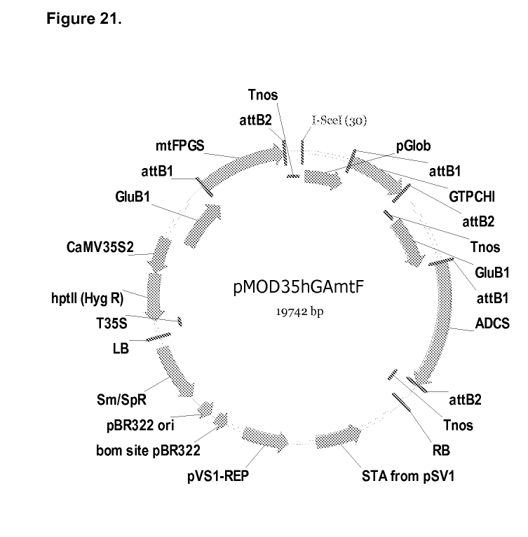

FIG. 22. Plasmid map of the plant transformation vector pMOD35hGAmtFsF carrying the folate genes for GTPCHI, ADCS, mitochondrial FPGS, and synthetic FBP. Sm/SpR Streptomycin/Spectinomycin resistance gene; hptII (Hyg R) hygromycin phosphotransferase gene (hygromycin resistance); GTPCHI Full-length cDNA for GTP cyclohydrolase I (At3g07270) from Arabidopsis; ADCS Full-length cDNA of ADC synthase (At2g28880) form Arabidopsis; mtFPGS Full-length cDNA for mitochondrial FPGS (AJ271786) from Arabidopsis; SFBP synthetic gene with adjusted codon bias coding for bovine FBP (P02702); GluB4 Promoter and 5′ untranslated region of rice GluB4 gene (AY427571); Tb4 3′-untranslated region and terminator of rice GluB4 gene, corresponds to the 9967-10722 of the AACV01003830 accession; STA from pSV1 STA region from pVS1 plasmid; bom site pBR322 bom site from pBR322; LB left border repeat from C58 T-DNA; attB1 Gateway recombination repeat; attB2 Gateway recombination repeat; RB right border T-DNA repeat; CaMV35S2 CaMV 35S promoter duplicated; pGlob Promoter and 5′ UTR of the seed storage 26 kDa alpha globulin from Oryza sativa (japonica), corresponds to 58-981 by of AY427575.1; GluB1 Promoter and 5′ UTR region of the GluB1 seed storage protein from Oryza sativa (japonica), corresponds to the 1130-2335 by of the AY427569 accession; pVS1-REP replication origin of pVS1; pBR322 ori origin pBR322; T35S transcriptional terminator of CaMV 35S; Tnos transcription terminator from NOS gene A. tumefaciens.

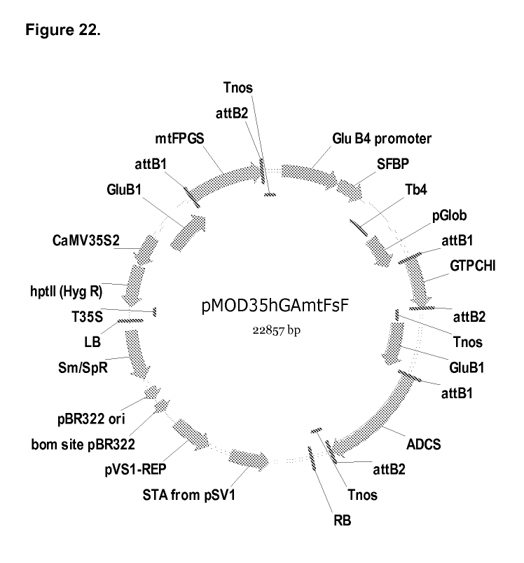

FIG. 23. Plasmid map of the plant transformation vector pMOD35hGAmtFCAsF carrying the folate genes for GTPCHI, ADCS, mitochondrial FPGS, and Arabidopsis carbonic anhydrase 2 (Fabre et al., 2007, Plant Cell Environ. 30, 617-629) fused with synthetic FBP. Sm/SpR Streptomycin/Spectinomycin resistance gene; hptII (Hyg R) hygromycin phosphotransferase gene (hygromycin resistance); GTPCHI Full-length cDNA for GTP cyclohydrolase I (At3g07270) from Arabidopsis; ADCS Full-length cDNA of ADC synthase (At2g28880) form Arabidopsis; mtFPGS Full-length cDNA for mitochondrial FPGS (AJ271786) from Arabidopsis; beta CA 2-FBP fusion Full-length arabidopsis β-carbonic anhydrase 2 cDNA (NM_{—}121478) in which a synthetic gene with adjusted codon bias coding for bovine FBP (P02702) was inserted in frame at the position 784; GluB4 Promoter and 5′ untranslated region of rice GluB4 gene (AY427571); Tb4 3′-untranslated region and terminator of rice GluB4 gene, corresponds to the 9967-10722 of the AACV01003830 accession; STA from pSV1 STA region from pVS1 plasmid; bom site pBR322 bom site from pBR322; LB left border repeat from C58 T-DNA; attB1 Gateway recombination repeat; attB2 Gateway recombination repeat; RB right border T-DNA repeat; CaMV35S2 CaMV 35S promoter duplicated; pGlob Promoter and 5′ UTR of the seed storage 26 kDa alpha globulin from Oryza sativa (japonica), corresponds to 58-981 by of AY427575.1; GluB1 Promoter and 5′ UTR region of the GluB1 seed storage protein from Oryza sativa (japonica), corresponds to the 1130-2335 by of the AY427569 accession; pVS1-REP replication origin of pVS1; pBR322 ori origin pBR322; T35S transcriptional terminator of CaMV 35S; Tnos transcription terminator from NOS gene A. tumefaciens.

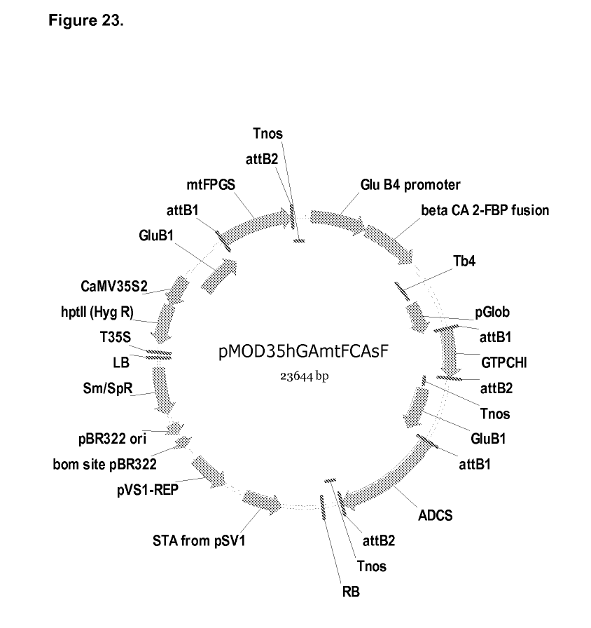

FIG. 24. Plasmid map of the plant transformation vector pMOD35hGAmtFB4sF carrying the folate genes for GTPCHI, ADCS, mitochondrial FPGS, and rice glutelin B4 (GluB4) storage protein (Qu and Takaiwa, 2004, Plant Biotechnology Journal 2, 113-125) fused with synthetic FBP. Sm/SpR Streptomycin/Spectinomycin resistance gene; hptII (Hyg R) hygromycin phosphotransferase gene (hygromycin resistance); GTPCHI Full-length cDNA for GTP cyclohydrolase I (At3g07270) from Arabidopsis; ADCS Full-length cDNA of ADC synthase (At2g28880) form Arabidopsis; mtFPGS Full-length cDNA for mitochondrial FPGS (AJ271786) from Arabidopsis; GluB4-SFBP fusion Full-length rice GluB4 cDNA (J090095N02) in which a synthetic gene with adjusted codon bias coding for bovine FBP (P02702) was inserted in frame between the positions 655 and 948; GluB4 Promoter and 5′ untranslated region of rice GluB4 gene (AY427571); Tb4 3′-untranslated region and terminator of rice GluB4 gene, corresponds to the 9967-10722 of the AACV01003830 accession; STA from pSV1 STA region from pVS1 plasmid; bom site pBR322 bom site from pBR322; LB left border repeat from C58 T-DNA; attB1 Gateway recombination repeat; attB2 Gateway recombination repeat; RB right border T-DNA repeat; CaMV35S2 CaMV 35S promoter duplicated; pGlob Promoter and 5′ UTR of the seed storage 26 kDa alpha globulin from Oryza sativa (japonica), corresponds to 58-981 by of AY427575.1; GluB1 Promoter and 5′ UTR region of the GluB1 seed storage protein from Oryza sativa (japonica), corresponds to the 1130-2335 by of the AY427569 accession; pVS1-REP replication origin of pVS1; pBR322 on origin pBR322; T35S transcriptional terminator of CaMV 35S; Tnos transcription terminator from NOS gene A. tumefaciens.

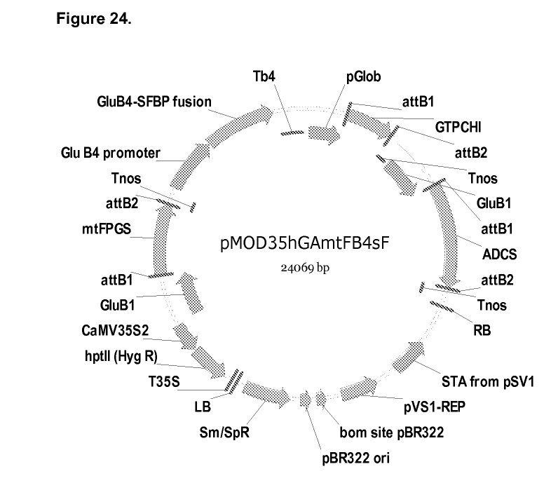

FIG. 25. Plasmid map of the plant transformation vector pMOD35hGActF carrying the folate genes for GTPCHI, ADCS, and cytosolic FPGS. Sm/SpR Streptomycin/Spectinomycin resistance gene; hptII (Hyg R) hygromycin phosphotransferase gene (hygromycin resistance); GTPCHI Full-length cDNA for GTP cyclohydrolase I (At3g07270) from Arabidopsis; ADCS Full-length cDNA of ADC synthase (At2g28880) form Arabidopsis; ctFPGS Full-length cDNA for cytosolic FPGS (AJ292545) from Arabidopsis; STA from pSV1 STA region from pVS1 plasmid; born site pBR322 bom site from pBR322; LB left border repeat from C58 T-DNA; attB1 Gateway recombination repeat; attB2 Gateway recombination repeat; RB right border T-DNA repeat; CaMV35S2 CaMV 35S promoter duplicated; pGlob Promoter and 5′ UTR of the seed storage 26 kDa alpha globulin from Oryza sativa (japonica), corresponds to 58-981 by of AY427575.1; GluB1 Promoter and 5′ UTR region of the GluB1 seed storage protein from Oryza sativa (japonica), corresponds to the 1130-2335 by of the AY427569 accession; pVS1-REP replication origin of pVS1; pBR322 ori origin pBR322; T35S transcriptional terminator of CaMV 35S; Tnos transcription terminator from NOS gene A. tumefaciens.

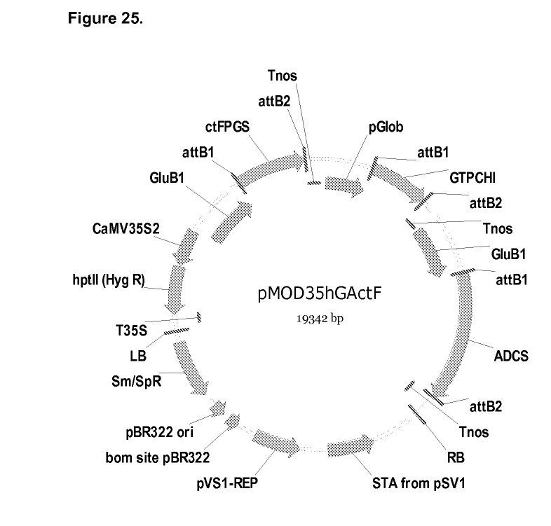

FIG. 26. Plasmid map of the plant transformation vector pMOD35hGActFsF carrying the folate genes for GTPCHI, ADCS, cytosolic FPGS and synthetic FBP. Sm/SpR Streptomycin/Spectinomycin resistance gene; hptII (Hyg R) hygromycin phosphotransferase gene (hygromycin resistance); GTPCHI Full-length cDNA for GTP cyclohydrolase I (At3g07270) from Arabidopsis; ADCS Full-length cDNA of ADC synthase (At2g28880) form Arabidopsis; ctFPGS Full-length cDNA for cytosolic FPGS (AJ292545) from Arabidopsis; SFBP synthetic gene with adjusted codon bias coding for bovine FBP (P02702); GluB4 Promoter and 5′ untranslated region of rice GluB4 gene (AY427571); Tb4 3′-untranslated region and terminator of rice GluB4 gene, corresponds to the 9967-10722 of the AACV01003830 accession; STA from pSV1 STA region from pVS1 plasmid; bom site pBR322 bom site from pBR322; LB left border repeat from C58 T-DNA; attB1 Gateway recombination repeat; attB2 Gateway recombination repeat; RB right border T-DNA repeat; CaMV35S2 CaMV 35S promoter duplicated; pGlob Promoter and 5′ UTR of the seed storage 26 kDa alpha globulin from Oryza sativa (japonica), corresponds to 58-981 by of AY427575.1; GluB1 Promoter and 5′ UTR region of the GluB1 seed storage protein from Oryza sativa (japonica), corresponds to the 1130-2335 by of the AY427569 accession; pVS1-REP replication origin of pVS1; pBR322 ori origin pBR322; T35S transcriptional terminator of CaMV 35S; Tnos transcription terminator from NOS gene A. tumefaciens.

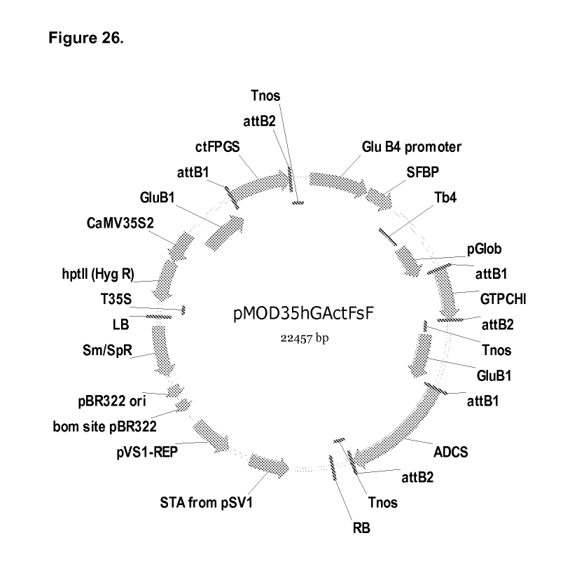

FIG. 27. Plasmid map of the plant transformation vector pMOD35hGActFCAsF carrying the folate genes for GTPCHI, ADCS, cytosolic FPGS and Arabidopsis carbonic anhydrase 2-synthetic FBP fusion. Sm/SpR Streptomycin/Spectinomycin resistance gene; hptII (Hyg R) hygromycin phosphotransferase gene (hygromycin resistance); GTPCHI Full-length cDNA for GTP cyclohydrolase I (At3g07270) from Arabidopsis; ADCS Full-length cDNA of ADC synthase (At2g28880) form Arabidopsis; ctFPGS Full-length cDNA for cytosolic FPGS (AJ292545) from Arabidopsis; beta CA 2-FBP fusion Full-length arabidopsis β-carbonic anhydrase 2 cDNA (NM_{—}121478) in which a synthetic gene with adjusted codon bias coding for bovine FBP (P02702) was inserted in frame at the position 784; GluB4 Promoter and 5′ untranslated region of rice GluB4 gene (AY427571); Tb4 3′-untranslated region and terminator of rice GluB4 gene, corresponds to the 9967-10722 of the AACV01003830 accession; STA from pSV1 STA region from pVS1 plasmid; born site pBR322 bom site from pBR322; LB left border repeat from C58 T-DNA; attB1 Gateway recombination repeat; attB2 Gateway recombination repeat; RB right border T-DNA repeat; CaMV35S2 CaMV 35S promoter duplicated; pGlob Promoter and 5′ UTR of the seed storage 26 kDa alpha globulin from Oryza sativa (japonica), corresponds to 58-981 by of AY427575.1; GluB1 Promoter and 5′ UTR region of the GluB1 seed storage protein from Oryza sativa (japonica), corresponds to the 1130-2335 by of the AY427569 accession; pVS1-REP replication origin of pVS1; pBR322 on origin pBR322; T35S transcriptional terminator of CaMV 35S; Tnos transcription terminator from NOS gene A. tumefaciens.

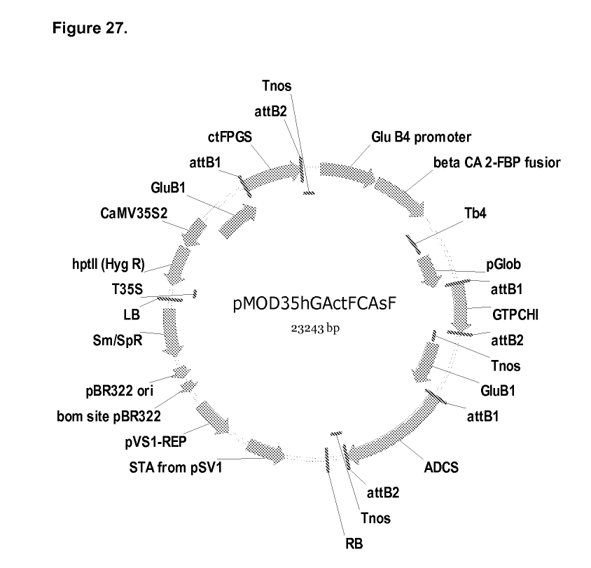

FIG. 28. Plasmid map of the plant transformation vector pMOD35hGActFB4sF carrying the folate genes for GTPCHI, ADCS, cytosolic FPGS and rice GluB4-synthetic FBP fusion. Sm/SpR Streptomycin/Spectinomycin resistance gene; hptII (Hyg R) hygromycin phosphotransferase gene (hygromycin resistance); GTPCHI Full-length cDNA for GTP cyclohydrolase I (At3g07270) from Arabidopsis; ADCS Full-length cDNA of ADC synthase (At2g28880) form Arabidopsis; ctFPGS Full-length cDNA for cytosolic FPGS (AJ292545) from Arabidopsis; GluB4-SFBP fusion Full-length rice GluB4 cDNA (J090095N02) in which a synthetic gene with adjusted codon bias coding for bovine FBP (P02702) was inserted in frame between the positions 655 and 948; GluB4 Promoter and 5′ untranslated region of rice GluB4 gene (AY427571); Tb4 3′-untranslated region and terminator of rice GluB4 gene, corresponds to the 9967-10722 of the AACV01003830 accession; STA from pSV1 STA region from pVS1 plasmid; born site pBR322 born site from pBR322; LB left border repeat from C58 T-DNA; attB1 Gateway recombination repeat; attB2 Gateway recombination repeat; RB right border T-DNA repeat; CaMV35S2 CaMV 35S promoter duplicated; pGlob Promoter and 5′ UTR of the seed storage 26 kDa alpha globulin from Oryza sativa (japonica), corresponds to 58-981 by of AY427575.1; GluB1 Promoter and 5′ UTR region of the GluB1 seed storage protein from Oryza sativa (japonica), corresponds to the 1130-2335 by of the AY427569 accession; pVS1-REP replication origin of pVS1; pBR322 ori origin pBR322; T35S transcriptional terminator of CaMV 35S; Tnos transcription terminator from NOS gene A. tumefaciens.

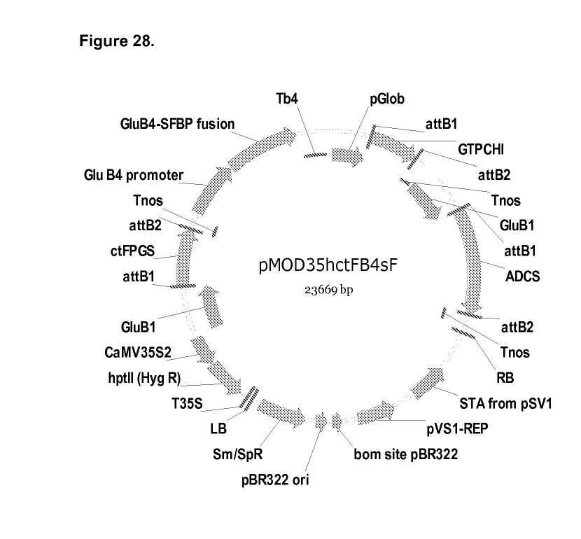

FIG. 29. Plasmid map of the plant transformation vector pMOD35hGAsF carrying the folate genes for GTPCHI, ADCS, and synthetic FBP. Sm/SpR Streptomycin/Spectinomycin resistance gene; hptII (Hyg R) hygromycin phosphotransferase gene (hygromycin resistance); GTPCHI Full-length cDNA for GTP cyclohydrolase I (At3g07270) from Arabidopsis; ADCS Full-length cDNA of ADC synthase (At2g28880) form Arabidopsis; SFBP synthetic gene with adjusted codon bias coding for bovine FBP (P02702); GluB4 Promoter and 5′ untranslated region of rice GluB4 gene (AY427571); Tb4 3′-untranslated region and terminator of rice GluB4 gene, corresponds to the 9967-10722 of the AACV01003830 accession; STA from pSV1 STA region from pVS1 plasmid; born site pBR322 born site from pBR322; LB left border repeat from C58 T-DNA; attB1 Gateway recombination repeat; attB2 Gateway recombination repeat; RB right border T-DNA repeat; CaMV35S2 CaMV 35S promoter duplicated; pGlob Promoter and 5′ UTR of the seed storage 26 kDa alpha globulin from Oryza sativa (japonica), corresponds to 58-981 by of AY427575.1; GluB1 Promoter and 5′ UTR region of the GluB1 seed storage protein from Oryza sativa (japonica), corresponds to the 1130-2335 by of the AY427569 accession; pVS1-REP replication origin of pVS1; pBR322 ori origin pBR322; T35S transcriptional terminator of CaMV 35S; Tnos transcription terminator from NOS gene A. tumefaciens.

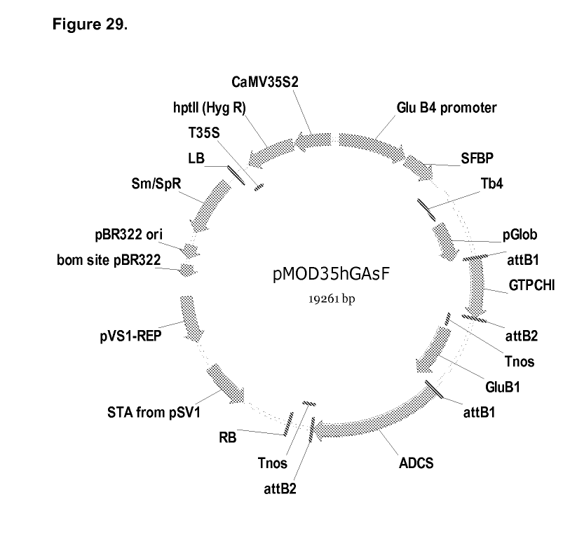

FIG. 30. Plasmid map of the plant transformation vector pMOD35hGACAsF carrying the folate genes for GTPCHI, ADCS, and Arabidopsis carbonic anhydrase 2-synthetic FBP fusion. Sm/SpR Streptomycin/Spectinomycin resistance gene; hptII (Hyg R) hygromycin phosphotransferase gene (hygromycin resistance); GTPCHI Full-length cDNA for GTP cyclohydrolase I (At3g07270) from Arabidopsis; ADCS Full-length cDNA of ADC synthase (At2g28880) form Arabidopsis; beta CA 2-FBP fusion Full-length arabidopsis β-carbonic anhydrase 2 cDNA (NM_{—}121478) in which a synthetic gene with adjusted codon bias coding for bovine FBP (P02702) was inserted in frame at the position 784; GluB4 Promoter and 5′ untranslated region of rice GluB4 gene (AY427571); Tb4 3′-untranslated region and terminator of rice GluB4 gene, corresponds to the 9967-10722 of the AACV01003830 accession; STA from pSV1 STA region from pVS1 plasmid; bom site pBR322 bom site from pBR322; LB left border repeat from C58 T-DNA; attB1 Gateway recombination repeat; attB2 Gateway recombination repeat; RB right border T-DNA repeat; CaMV35S2 CaMV 35S promoter duplicated; pGlob Promoter and 5′ UTR of the seed storage 26 kDa alpha globulin from Oryza sativa (japonica), corresponds to 58-981 by of AY427575.1; GluB1 Promoter and 5′ UTR region of the GluB1 seed storage protein from Oryza sativa (japonica), corresponds to the 1130-2335 by of the AY427569 accession; pVS1-REP replication origin of pVS1; pBR322 ori origin pBR322; T35S transcriptional terminator of CaMV 35S; Tnos transcription terminator from NOS gene A. tumefaciens.

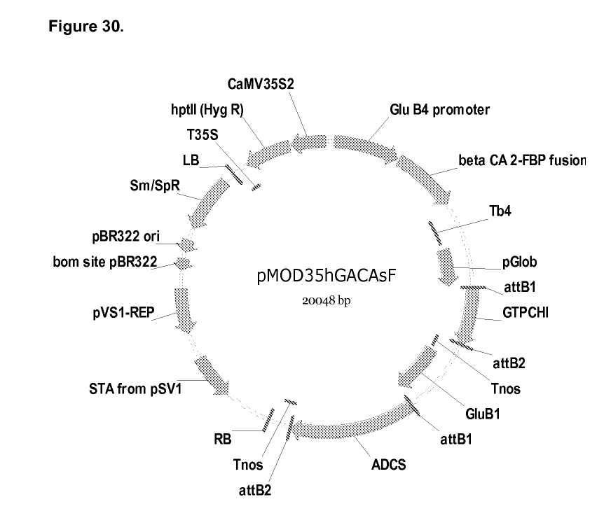

FIG. 31. Plasmid map of the plant transformation vector pMOD35hGAB4sF carrying the folate genes for GTPCHI, ADCS, rice GluB4-synthetic FBP fusion. Sm/SpR Streptomycin/Spectinomycin resistance gene; hptII (Hyg R) hygromycin phosphotransferase gene (hygromycin resistance); GTPCHI Full-length cDNA for GTP cyclohydrolase I (At3g07270) from Arabidopsis; ADCS Full-length cDNA of ADC synthase (At2g28880) form Arabidopsis; GluB4-SFBP fusion Full-length rice GluB4 cDNA (J090095N02) in which a synthetic gene with adjusted codon bias coding for bovine FBP (P02702) was inserted in frame between the positions 655 and 948; GluB4 Promoter and 5′ untranslated region of rice GluB4 gene (AY427571); GluB4 Promoter and 5′ untranslated region of rice GluB4 gene (AY427571); Tb4 3′-untranslated region and terminator of rice GluB4 gene, corresponds to the 9967-10722 of the AACV01003830 accession; STA from pSV1 STA region from pVS1 plasmid; bom site pBR322 bom site from pBR322; LB left border repeat from C58 T-DNA; attB1 Gateway recombination repeat; attB2 Gateway recombination repeat; RB right border T-DNA repeat; CaMV35S2 CaMV 35S promoter duplicated; pGlob Promoter and 5′ UTR of the seed storage 26 kDa alpha globulin from Oryza sativa (japonica), corresponds to 58-981 by of AY427575.1; GluB1 Promoter and 5′ UTR region of the GluB1 seed storage protein from Oryza sativa (japonica), corresponds to the 1130-2335 by of the AY427569 accession; pVS1-REP replication origin of pVS1; pBR322 ori origin pBR322; T35S transcriptional terminator of CaMV 35S; Tnos transcription terminator from NOS gene A. tumefaciens.

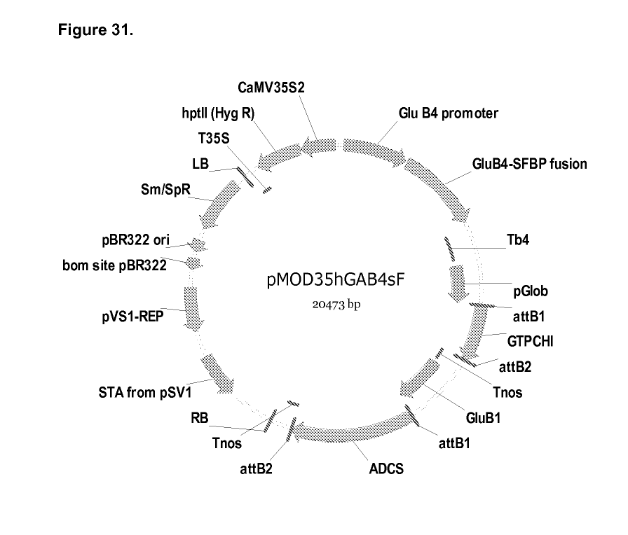

Table 1. Folate content in selected crops. Values, expressed as nmol of equivalent folic acid g⁻¹ of an edible portion (1 nmol/g corresponds approximately to 45 μg/100 g), were calculated from data published by USDA, 2006 (USDA National Nutrient Database for Standard Reference. http://www.nal.usda.qov/fnic/foodcomp/search/)

Table 2. Compound parameters for pterins.

Table 3. Source parameters for pterin determination.

Table 4. Analysis of p-ABA, pteridines, and folate content in seeds of transgenic rice plants transformed with A, G, GA constructs.

Table 5. Total folate levels in plants transformed with constructs with different folate biosynthesis genes combinations. The seeds from which the folate content has been determined have been stored over a long term. Therefore, it is possible that the folate level of these seeds, if treated as those in Table 4, are higher. As indicated before, for long term storage folate stabilization may be advisable. However, Table 5 clearly indicates that increase of folate may be obtained using constructs with different folate biosynthesis gene combinations.

Table 6. Percentages of identical nucleotides (amino acids) calculated from global pair-wise alignments of GTPCHI cDNAs (proteins) using GAP program at http://genome.cs.mtu.edu/align/align.html. Percentages for amino acid sequences are in parenthesis. Percentages for maize GTPCHI might be overestimated because only a partial sequence of the cDNA (protein) is available, thus, the other sequences had to be tailored to match the size. Comparison of each N-terminal and C-terminal parts of the plant enzyme to the bacterial or mammalian enzyme was done separately using BLAST algorithm. In the table 2 numbers represent similarities of N-terminal and C-terminal domains, respectively. Only matching parts were compared, while gaps and un-matching sequences were not accounted for. However, in case of the Arabidopsis to rice as well as E. coli to mouse comparisons, the full-length protein sequences were used. “-”, no significant similarity is found.

### TABLES

TABLE 1
Folate content in selected crops.

| | Folate content, |
|---|---|
| Crop | nmol/g edible portion |
| Rice (white, raw) | 0.13-0.18 |
| Wheat (hard, white, raw) | 0.84-0.95 |
| Maize (yellow, seeds, raw) | 0.42 |
| Tomato (fruits) | 0.20-0.64 |
| Peas (green, raw) | 1.45 |
| Spinach (leaves, raw) | 4.31 |
| Beans (pink, mature seeds, raw) | 10.28 |

TABLE 2
Compound parameters for pterins

| | Precursor | Product | | | | |
|---|---|---|---|---|---|---|
| | ion | ion | DP^{a} | EP^{a} | CXP^{a} | CE^{a} |
| | (m/z) | (m/z) | (V) | (V) | (V) | (V) |
| Neopterin | 252.0 | 191.9 | −55 | −8 | −11 | −12 |
| | 252.0 | 146.90 | −55 | −14 | −11 | −34 |
| DihydroNeopterin | 254.0 | 193.8 | −45 | −6 | −11 | −14 |
| | 254.0 | 193.8 | −45 | −10 | −9 | −24 |
| Hydroxymethyl | 192.0 | 162.1 | −55 | −10 | −7 | −24 |
| pterin | 192.0 | 118.8 | −55 | −8 | −7 | −32 |
| Hydroxymethyl | 194.1 | 138.0 | −55 | −8 | −7 | −16 |
| Dihydropterin | 194.1 | 164.0 | −55 | −8 | −7 | −20 |

^{a}DP: declustering potential; EP: entrance potential; CXP: collision cell exit potential; CE: collision energy; V: volt

TABLE 3
Source parameters pterin determination

| Temperature | Curtain | | | | | |
|---|---|---|---|---|---|---|
| (° C.) | gas | Gas 1 | Gas 2 | IS^{a} | IHE^{a} | CAD^{a} |
| 600° C. | 20 psi | 80 psi | 90 psi | −4500 V | on | 7 psi |

^{a}IS: ionspray voltage; IHE: interface heater; CAD: collision activated dissociation gas

TABLE 4
Analysis of p-ABA, pteridines, and folate content in seeds of
transgenic rice plants transformed with A, G, GA constructs.

| Transgenic | Pterins, | SD, | pABA, | SD, | Folate, | SD, |
|---|---|---|---|---|---|---|
| line | nmol/g | nmol/g | nmol/g | nmol/g | nmol/g | nmol/g |
| WT | 0.05 | 0.03 | nd^{a} | nd | 0.42 | 0.02 |
| V | 0.05 | 0.02 | nd | nd | 0.36 | 0.02 |
| G 17.1 | 0.58 | 0.01 | nd | nd | 0.40 | 0.06 |
| G 24.1 | 1.57 | 0.42 | nd | nd | 0.42 | 0.06 |
| G 25.2 | 1.47 | 0.04 | nd | nd | 0.49 | 0.05 |
| A 11.2 | nd | nd | 13.45 | 0.25 | 0.12 | 0.00 |
| A 12.1 | nd | nd | 10.32 | 2.68 | 0.19 | 0.11 |
| A 25.3 | nd | nd | 28.59 | 4.77 | 0.07 | 0.01 |
| A 49.6 | nd | nd | 10.73 | 4.36 | 0.07 | 0.00 |
| A 51.1 | nd | nd | 13.55 | 0.17 | 0.08 | 0.02 |
| GA 29.4 | 0.21 | 0.01 | 6.33 | 0.37 | 16.67 | 3.64 |
| GA 9.15 | 0.19 | 0.06 | 9.57 | 0.75 | 38.30 | 0.16 |
| GA 4.4 | 0.07 | 0.02 | 7.14 | 1.20 | 8.00 | 0.05 |
| GA 26.5 | 0.46 | 0.21 | 5.80 | 0.72 | 12.02 | 4.53 |
| GA 17.8 | 0.20 | 0.01 | 12.80 | 2.72 | 21.66 | 9.28 |
| GA 19.12 | 0.24 | 0.01 | 5.58 | 1.67 | 8.95 | 4.06 |

TABLE 5
Total folate levels in plants transformed with constructs
with different folate biosynthesis genes combinations.

| Construct | Folate, nmol/g | SD, nmol/g |
|---|---|---|
| WT | 0.135 | 0.023 |
| GADL 28.1 | 1.46 | 0.91 |
| GADF 65-2-12 | 4.31 | 1.66 |

TABLE 6
GTPCHI sequence comparison.

| | SEQ ID | SEQ ID | SEQ ID | SEQ ID | SEQ ID | SEQ ID | SEQ ID |
|---|---|---|---|---|---|---|---|
| | NO: 1(2) | NO: 3(4) | NO: 5(6) | NO: 7(8) | NO: 9(10) | NO: 59(60) | NO: 61(62) |
| | A. thaliana | tomato | rice | wheat | maize | E. coli | mouse |
| SEQ ID | 100(100) | 60(61) | 41(46) | 43(49) | 40(44) | —(27-28) | —(34-40) |
| NO: 1(2) | | | | | | | |
| A. thaliana | | | | | | | |
| SEQ ID | | 100(100) | 43(52) | 43(53) | 48(48) | —(31-26) | —(39-36) |
| NO: 3(4) | | | | | | | |
| tomato | | | | | | | |
| SEQ ID | | | 100(100) | 83(83) | 81(81) | —(29-31) | —(37-38) |
| NO: 5(6) | | | | | | | |
| rice | | | | | | | |
| SEQ ID | | | | 100(100) | 78(80) | —(29-30) | —(33-36) |
| NO: 7(8) | | | | | | | |
| wheat | | | | | | | |
| SEQ ID | | | | | 100(100) | —(37-30) | —(37-36) |
| NO: 9(10) | | | | | | | |
| maize | | | | | | | |
| SEQ ID | | | | | | 100(100) | —(36) |
| NO: 59(60) | | | | | | | |
| E. coli | | | | | | | |
| SEQ ID | | | | | | | 100(100) |
| NO: 61(62) | | | | | | | |
| mouse | | | | | | | |

### MODES FOR CARRYING OUT THE INVENTION

### Example 1

Microbial strains and plant material. Escherichia coli strains DH-5α and DB3.1™ (Invitrogen) were used for plasmid manipulations and propagation of “empty” Gateway™ vectors, respectively. Agrobacterium tumefaciens strain LBA 4404 was used for delivery of T-DNA from binary vectors into plant cells. Japonica rice (Oryza sativa L.) variety Nipponbare plants were grown in soil under 8 h of light (420 μmoles/m²·s light intensity, 28° C., 80% humidity) and 16 h darkness (21° C., 80% humidity) regime. As a starting material for the Agrobacterium-mediated rice transformation somatic embryogenic calli were used. The calli were produced on mature rice embryos as described in Rueb et al. (1994, Plant Cell Tiss. Org., 36:259-264). The transformation was performed according to Scarpella and co-workers (Scarpella et al., 2000, Development 127:3655-3669).

Molecular cloning and construct design. Full-length cDNAs of Arabidopsis GTPCHI and ADCS flanked by Gateway™ attB recombination sites were amplified by RT-PCR from total Arabidopsis RNA using a kit (Invitrogen) and the following primer pairs: 5′-AAAAAGCAGGCTCTACCATGGGCGCATTAGATGAGGGA-3′ (SEQ ID NO:25), 5′-AGAAAGCTGGGTCTTAGTTCTTTGAACTAGTGTTTCGCTG-3′ (SEQ ID NO:26) for GTPCHI and 5′-AAAAAGCAGGCTCTAAACGAGTTATGAACATGAAT-3′ (SEQ ID NO:27), 5′-AGAAAGCTGGGTAAAACTATTGTCTCCTCTGATCACT-3′ (SEQ ID NO:28) for ADCS. Sequences of the Arabidopsis GTPCHI and ADCS are publically available under GenBank accessions AF489530 and AY096797, respectively. The cDNAs were recombined with the pDONR201 vector (Invitrogen) according to the Gateway™ manual (Invitrogen) resulting in pGTPCH201 and pADCS201 entry clones, respectively. Sequences of both cloned cDNAs were verified by DNA sequencing of the entry clones.

Binary plant transformation vectors were designed based on a modular plant vector transformation system (Goderis, I. et al., 2002, Plant. Mol. Biol. 50, 17-27). A rice globulin promoter-nopaline synthase transcription terminator (Tnos) cassette was cloned into pAUX3132 auxiliary vector resulting in the pGlob32 vector suitable for placing genes under the control of the rice globulin promoter. Similarly, a pGluB-1-Tnos expression cassette was cloned into pAUX3133 auxiliary vector giving rise to the pGluB133 vector. Both constructs were converted into the Gateway™ destination vectors by using the Gateway™ vector conversion kit (Invitrogen) resulting in pGlob32-Gate and pGluB133-Gate constructs. The kanamycin selection marker (nptII gene under the control of Pnos promoter) was removed from the pMODUL3409 plant transformation vector using Ascl and replaced by hptII gene (hygromycin selection marker) under the control of 35S promoter. The latter was amplified from the pCAMBIA1304 vector with primers 5′-AAGGCGCGCCACACTCTCGTCTACTCCAAGAA-3′ (SEQ ID NO:29) and 5′-CAGGCGCGCCGATCTGGATTTTAGTACTGGAT-3′ (SEQ ID NO:30) containing AscI recognition sites, resulting in the pMOD35h plant transformation vector.

pGTPCH201 and pADCS201 entry clones were recombined with pGlob32-Gate and pGluB133-Gate destination vectors, respectively, according to the Gateway™ protocol (Invitrogen) resulting in pGTPCH32 and pADCS33 expression vectors. Glob promoter-GTPCHI-Tnos expression cassette was cut out from pGTPCH32 with I-CeuI homing endonuclease (New England Biolabs) and cloned into the pMOD35h vector using the corresponding sites resulting in the pMOD35hG plant transformation vector (G construct). Furthermore, the pGluB1-ADCS-Tnos expression cassette was cloned into the pMOD35hG vector using PI-PspI homing endonuclease (New England Biolabs Ipswich, Mass.) resulting in the pMOD35hGA (GA construct) plant transformation vector. Finally, the GHTPCHI-expression unit was removed from pMOD35hGA by cutting with I-CeuI and re-ligating the vector resulting in the pMOD35hA (A construct) plant transformation vector.

All cloning procedures were designed and simulated in silico with Vector NTI Advance software (Invitrogen).

Southern and Northern blotting-hybridizations. Rice genomic DNA was isolated from leaves of fully developed soil-grown plants using Invisorb Spin Plant DNA Mini Kit (Invitek GmbH, Berlin, Germany). Total rice seed RNA was isolated using Trizol™ reagent (Invitrogen) according to the manufacturer's instructions with minor modifications.

Hybridizations were on Hybond+membranes (Amersham Biosciences) according to the manufacturer's instructions. A PCR amplified fragment of GTPCHI cDNA (using the primers 5′-ATAACCATGGGCGCATTAGATGAGGGATGT-3′ (SEQ ID NO:31) and 5′-AT AACTAGTAAATGGAGAGCTTGACTCTGTCTT-3′) (SEQ ID NO:32) as well as an EcoRI-HindIII restriction fragment of ADCS cDNA cut from the A-construct were used as probes.

Real time PCR. Real time PCR was based on a duplex TaqMan® assay (Livak et al., 1995, PCR Methods Appl. 4:357-362) (Applied Biosystems, Foster City, Calif.). Primers and probes were designed using Beacon Designer software (PREMIER Biosoft International) and synthesized by Sigma-Aldrich. The rice sucrose phosphate synthase gene (SPS) (accession number U33175) (primers 5′-CCTCCGGTGCCATGAACAAG-3′

(SEQ ID NO:33) and 5′-ACAGCCCTGAACACCTCCTG-3′ (SEQ ID NO:34)), probe 5′-HEX-CTCCTCCGCCGACG CCGCAG-BHQ2-3 (SEQ ID NO:35)) was used as an internal reference; while the hptII gene (hygromycin selection marker on T-DNA) (primers 5′-AGGGTGTCACGTTGCAAGAC-3′ (SEQ ID NO:36) and 5′-CGCTCGTCTGGCT AAGATCG-3′ (SEQ ID NO:37), probe 5′-FAM-TGCCTGAAACCGAACTGCCCGCTG-BHQ1-3′ (SEQ ID NO:38)) was chosen for copy number quantification. Q-PCR was carried out on a RotorGene-3000 real time PCR machine (Corbett Life Science) using Absolute QPCR Mix (ABgene). Threshold cycle determination was done using software from the supplier. For copy number calculations, the 2^{-ΔΔCt} method was used (Livak and Schmittgen Methods 25, 402, 2001).

p-ABA and Folate Analysis.

Chemicals and Reagents. pABA and its internal standard 3-NH₂-4-CH₃-benzoic acid were from Sigma (Bornem, Belgium). 5-Methyltetrahydrofolate (5-MTHF), 10-formylfolic acid (10-CHOFA), 5,10-methenyltetrahydrofolate (5,10-CH⁺THF), neopterin (NeoP), dihydroneopterin (NeoDP), hydroxymethylpterin (HMP) and hydroxymethyldihydropterin (HMDP) were purchased from Schirck's Laboratories (Jona, Switzerland). Folic acid (FA), tetrahydrofolate (THF) and 5-formyltetrahydrofolate (5-CHOTHF) were from Sigma (Bornem, Belgium). (65)-5-CH₃—H₄Pte[¹³C₅]Glu-Ca ([¹³C₅]5-MTHF), (6S)—H₄Pte[¹³C₅]Glu ([¹³C₅]THF), (6R)-5,10-CH⁺—H₄Pte[¹³C₅]Glu-Cl.HCl ([¹³C₅]5,10-CH⁺THF) and Pte[¹³C₅]Glu (free acid, [¹³C₅]FA) were used as internal standard (1S) (Merck Eprova AG, Switzerland, labelling yield >99%). Based on retention characteristics of the liquid chromatographic method, [¹³C₅]5-MTHF was used as IS for 5-MTHF, [¹³C₅]THF for THF, [¹³C₅]5,10-CH⁺THF for 5-10-CH⁺THF, while [¹³C₅]FA was used as internal standard for FA, 10-CHOFA and 5-CHOTHF. The IS were used for compensation of the variation of instrument sensitivity.

All folate stock solutions, i.e. THF, 5-MTHF, 10-CHOFA, FA, 5-CHOTHF, 5,10-CH⁺THF with a concentration of 100 μg/mL, were prepared in 50 mM phosphate buffer (pH 7.0) containing 1% of ascorbic acid and 0.5% of dithiothreitol (DTT)/methanol (50/50, v/v). All standards and internal stock solutions were stored at −80° C. No degradation was observed after storage during 6 months under these conditions. pABA stock solutions contained 1 mg/ml of pABA in water and were stored at 4° C. Pterin stock solutions (100 μg/mL) were prepared in 50-mM phosphate buffer (pH 7.0) containing 1% of ascorbic acid and 0.5% of dithiothreitol (DTT)/methanol (25/75, v/v). The pterine stock solutions were stored at −80° C.

LC-MS grade water, acetonitrile and methanol were obtained from Biosolve (Valkenswaard, The Netherlands). Formic acid, ammonium bicarbonate, sodium phosphate, ascorbic acid, dithiotreitol (DTT) and other reagents were of high purity grade and were either purchased from VWR (Leuven, Belgium) or Sigma (Bornem, Belgium).

Mass spectrometric instrumentation and settings. All experiments were performed by electrospray ionization utilizing heated auxiliary gas in the multiple reaction monitoring (MRM) mode on an Applied Biosystems API 4000 tandem quadrupole mass spectrometer (Foster City, Calif., USA), operated in the positive ionization mode with the Analyst 1.4 controlling software. Source and compound-specific parameters of pABA and folates were determined previously (De Brouwer et al., 2007, Phytochem. Anal., in press; Zhang et al., 2005, Rapid Commun Mass Spectrom 19: 963-969; Zhang et al., 2005, J Chromatogr A 1078: 59-66). The compound parameters and source conditions for pterins are listed in Tables 2 and 3, respectively (see below).

HPLC Conditions. The HPLC system is an Agilent 1100 (Palo Alto, Calif., USA) including a quaternary pump (flow rate 1.0 mL/min), an autosampler, column oven, and degasser. The needle wash solvent was a mixture of methanol/water (50/50, v/v).

For folate determinations a Purospher Star RP-18 end-capped column (150 mm×4.6 mm I.D.; octadecylsilyl, 5-μm particle size from Merck, Darmstadt, Germany), and a guard column RP 18 (4 mm×4 mm I.D.; octadecylsilyl, 5-μm particle size from Merck, Darmstadt, Germany) were used as well as a Polaris 3C18-A (150 mm×4.6 mm I.D.; 3-μm particle size from Varian) with pre column. In both cases the mobile phase consisted of eluent A (0.1% of formic acid in water) and eluent B (0.1% of formic acid in acetonitrile). For the Purospher Star column the conditions were as in Zhang et al., 2005 (J Chromatogr A 1078: 59-66). The starting eluent with the Polaris column was 95% A/5% B, which was held for 2 minutes. Next, the proportion of B was increased linearly to 15% in 1 min and then to 25% in 2 min. The proportion of B was then immediately increased to 100% and kept for 5 min. Afterwards the mobile phase was immediately adjusted to its initial composition and held for 8 min in order to re-equilibrate the column. The injection volume was 20 μL. The column was kept at 25° C. in a column oven. The autosampler (kept at 4° C.) was equipped with a black door avoiding samples to be exposed to light.

For pABA determination the same Purospher Star RP-18 column was used as for folate determination. Conditions can be found in our previous work (Zhang et al., 2005, Rapid Commun Mass Spectrom 19: 963-969).

The separation of pterins was performed on an Atlantis T3 column (150 mm×4.6 mm; 3 μm particle size, from Waters) and a guard column RP 18 (20 mm×4.6 mm I.D.; 3 μm particle size also from Waters) at 25° C. with a flow rate of 0.8 mL/min. The mobile phase used was 2 mM ammonium bicarbonate in water, pH 4.6 (A) and 2 mM ammonium bicarbonate in ACN/water (95/5, v/v), pH 4.6 (B) under gradient conditions. The gradient started at 1% of B, it was raised linearly to 35% B in 7 minutes. Subsequently the mobile phase was programmed to 100% of B over 3 minutes, to rinse the column, before re-equilibrating the column for 8 minutes.

Sample preparation. Typically, 10 mature rice seeds were collected, manually de-husked and polished overnight in a Petri dish fitted with fine sand paper on a rotary shaker at 1000 rpm. Embryos were manually removed from the endosperm. The seeds were then transferred to a 2 ml Eppendorf tube and incubated for 25 min at 95° C. in 0.25 ml of 1% ascorbate or 0.5% DTT solution for pABA or pterin determinations, respectively. Subsequently, 1 ml methanol was added, the tubes were cooled on ice and after addition of 5 mm stainless steel balls, the samples were ground at room temperature on a Retsch Mill (Retsch) at a frequency of 30 rps for 1 hour.

For pABA determination, 1 ml of MeOH and IS were added and rotated for 20 min. This was followed by centrifugation at 1200 g for 25 minutes, the supernatant was transferred in a 15 mL tube and centrifugation was repeated. Subsequently, both supernatants were combined. Further sample preparation was as described (Zhang et al., 2005, Rapid Commun Mass Spectrom 19: 963-969).

Similarly, for determination of pterins, 1 ml of MeOH and IS were added, rotated for 20 min on a Labinco rotary mixer (Labservice, Kontich, Belgium) and centrifuged at 1200×g for 25 minutes. The supernatant was transferred to another tube. Another 2 mL of MeOH was added to the residue for a second extraction. After another round of rotation and centrifugation, the supernatants were combined. The methanolic layer was dried completely under nitrogen gas at 35° C. A 0.2 mL aliquot of water (with 0.5% of DTT) was added followed by sonication for 5 min. To release conjugated pterins, 25 μL of 2M HCl was added. After capping, the tube was incubated at 80° C. for 1 h. After cooling down the solution, 25 μL of 2 M NaOH was added for neutralization. Finally, the samples were centrifuged at 10 000×g for 30 min on a 5 kDa molecular weight (MW) cut off membrane filter (Millipore) before LC-MS/MS analysis.

Sample preparation for folates was based on our method for pABA analysis in plants (Zhang et al., 2005, J Chromatogr A 1078: 59-66). However, some modifications were necessary to homogenize the samples and tri-enzyme treatment was utilized to enhance the recovery of folates from rice seeds. One mL of phosphate buffer (which contained the 4 internal standards, 1% ascorbic acid, 0.5% of DTT at pH 7) was added to 10 rice seeds and this mixture was incubated at 95° C. for 15 minutes. After cooling down, the rice was homogenized as above 10 μL of amylase and 500 μL of buffer were added (to avoid a sticky solution). After 10 minutes this reaction was stopped by addition of 150 μL of protease (5.3 units/mg solid, Sigma). The tube was kept at 37° C. for 1 hour for incubation. The capped tube was placed at 100° C. for 10 minutes to stop the enzymatic reaction. Further sample preparation was as described (Zhang et al., 2005, J Chromatogr A 1078: 59-66).

### Example 2

### Overexpression of Arabidopsis GTPCHI and ADCS in Rice Seeds

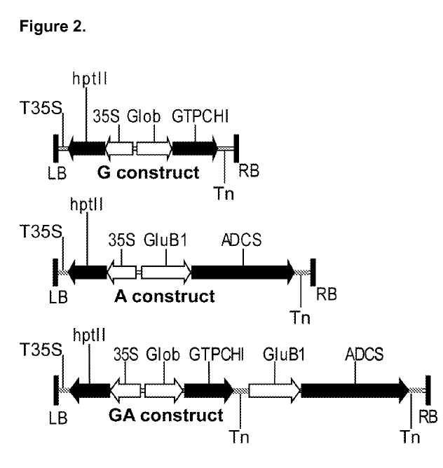

In order to compare the effect of engineering of the two branches of folate biosynthesis, 3 plant transformation vectors were constructed (FIG. 2). The G-vector contained a cDNA of Arabidopsis GTPCHI, under the control of the rice endosperm-specific globulin (Glob) promoter (Nakase et al., 1996, Gene 170:223-226). The A-construct contained Arabidopsis ADCS cDNA under the control of the rice endosperm-specific glutelin B1 (GluB1) promoter (Takaiwa et al., 1991, Plant. Mol. Biol. 17:875-885). The GA-construct combined both G and A expression cassettes on a single T-DNA.

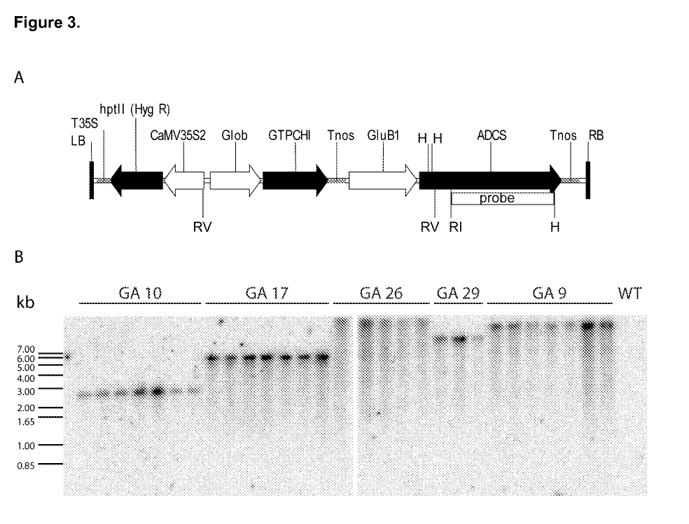

All 3 vectors were stably introduced in the genome of Nipponbare japonica rice by means of Agrobacterium-mediated transformation. Empty vector (V) was used as a transformation control. Fifty one, 48 and 67 primary transformed lines (T0) were generated for A, G and GA constructs, respectively, in 3 transformation experiments. The transgenic plants had a phenotype indistinguishable from the wild type throughout development and had similar seed-set capabilities. To screen for single copy transformation events, genomic DNA was isolated from T0 plants and the T-DNA copy number was quantified using real time PCR (Q-PCR). A hygromycin resistant single copy line for which hemizygosity was first proven by Southern blotting, was used as a single copy per diploid genome standard. T1 progenies obtained by self-pollination of each of these lines were also studied by Q-PCR and homozygous single copy T-DNA insertions were identified as those containing a double amount of the transgene as compared to hemizygous parental T0 plants. For T1 progenies of several GA transgenic lines, single copy insertions were also confirmed by Southern hybridization (FIG. 3).

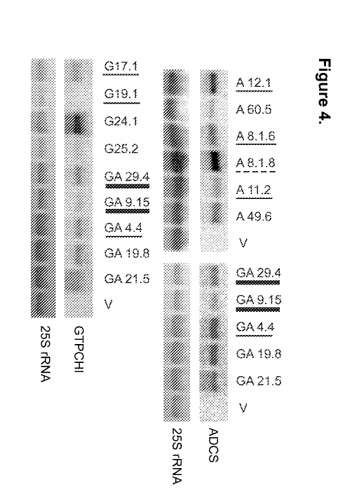

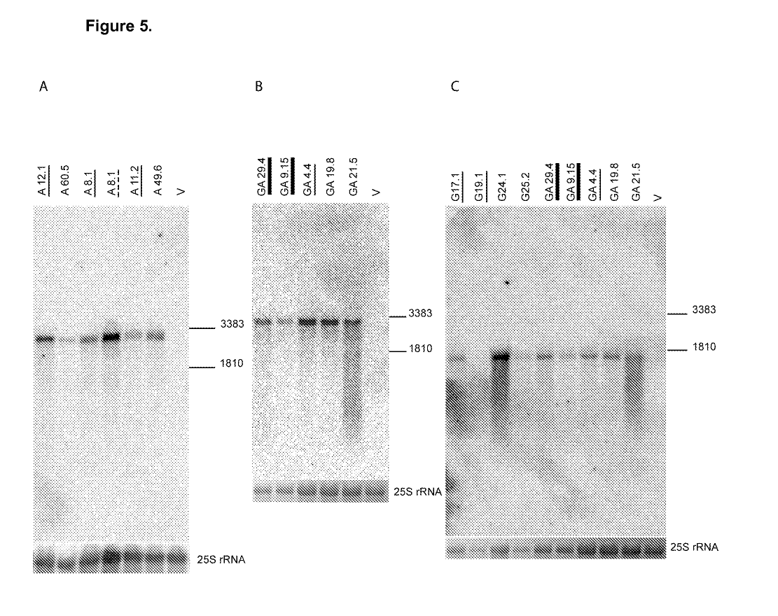

Expression of GTPCHI, ADCS or both transgenes in T2 seeds of G-, A-, and in T2 and T3 seeds of GA-transgenic lines, respectively, was confirmed by northern hybridization with the corresponding probes (FIG. 4 and FIG. 5).

Homozygous single T-DNA copy T2 or T3 seeds and T2 seeds of lines with multiple T-DNA insertions of A-, G-, and GA-transgenic plants, which produced a sufficient amount of seeds, were used for analysis of folates, pteridines and p-ABA.

### Example 3

### Production of Seeds of A- and G-Lines

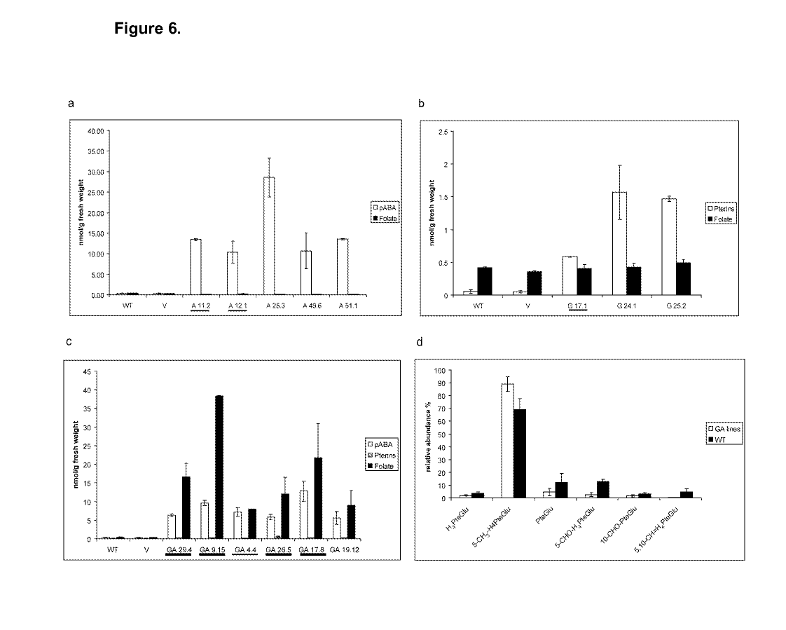

Analysis of transgenic A-lines showed substantially elevated total p-ABA levels, on average 49 times higher than in seeds of control plants (15.3±7.6 and 0.32±0.03 nmol/g, respectively) (FIG. 6A). The high level of p-ABA accumulation demonstrates functionality of the overexpressed ADCS and efficient enhancement of the p-ABA branch of folate biosynthesis in seeds of transgenic A-lines. The total folate content in all A-lines analyzed was up to 6 times lower as compared to that of wild type seeds or seeds of control plants transformed with the empty vector (FIG. 6A).

Seeds of transgenic lines overexpressing GTPCHI did not show a substantial difference in folate levels as compared to control plants. However, analysis of pterin biosynthesis intermediates and their oxidized forms (hydroxymethyldihydropterin (HMDHP), dihydroneopterin (DHN), hydroxymethylpterin (HMP) and neopterin) revealed a considerable pterin accumulation (on average 25-fold as compared to the control) in seeds of GTPCHI overexpressing lines (FIG. 6B), confirming enhancement of the pteridine branch in G-lines. The most abundant pterin was neopterin (69.9%±12.6%) followed by HMP (25.9%±11.6%) and DHN (5.4%±1.8%), while HMDHP was undetectable.

### Example 4

### Massive Accumulation of Folates in Seeds of GA-Plants

In contrast to A- and G-lines, seeds of the GA transgenic lines demonstrated massive folate accumulation (FIG. 6C). The amount of total folate in the transgenic lines ranged from 6.0 up to 38.3 nmol/g, which is 15-100 times higher than in the wild type or in plants transformed with the empty vector (FIG. 6C). The fact that overexpression of the 2 first enzymes of pathway leads to high folate accumulation implies that either all downstream enzymatic activities are present and sufficient to sustain the high folate production or that they are induced by the products of GTPCHI and ADCS activities or both. Remarkably, all GA-lines accumulated relatively high amounts of p-ABA (FIG. 6C), despite the fact that overproduced p-ABA was directed to folate biosynthesis. This indicates that the flux through the pABA branch is not saturated and hence, that further engineering of the pterin branch might result in even more elevated folate levels. The amount of pABA was on average 2 times lower than in A-lines (7.8±2.8 and 15.3±7.6 nmol/g⁻¹, respectively).

The level of pterins was on average 5.5 times lower in the seeds of GA-lines (0.23±0.12 nmol/g) as compared to G-lines (1.23±0.54 nmol/g), albeit still 4 times higher than in the wild type or empty vector-transformed seeds.

5-Methyltetrahydrofolate is the major folate in folate overproducing seeds. Analysis of folate derivatives has shown that the major folate fraction in GA transgenic plants was represented by 5-methyltetrahydrofolate, which on average accounted for up to 89% of total folate (FIG. 6d). 5-Methyltetrahydrofolate is also the major folate in seeds of wild type Nipponbare plants, representing around 69% of total folates therein.

### Example 5

### Massive Folate Accumulation with Minimal Pterin Accumulation: a Healthy Food Product

In the above presented examples, a GTPCHI of plant origin (Arabidopsis) has been chosen for the enhancement of the pteridine branch of the pathway rather than the mammalian or bacterial enzyme in order to analyse the effect of the increased expression of the plant GTPCHI on folate production in a plant cell.

Unexpected, low folate content in pABA-enhanced A-lines has revealed an interesting new insight into potential regulation of the folate biosynthesis pathway in plant: a yet unidentified inhibitory effect of high p-ABA concentrations on folate biosynthesis in the absence of elevated levels of dihydroneopterin, which must be condensed with p-ABA in the following biosynthesis step. In spite of the fact that all GA-lines accumulated relatively high amounts of p-ABA, these amounts were 4.5-10 times lower than in folate biofortified tomato produced by Hanson's group. The limited increase of p-ABA content in biofortified rice should not be of much concern from a nutritional point of view because p-ABA is listed as GRAS (Generally Regarded As Safe) compound by the Federal Drug Administration (http://www.cfsan.fda.gov/˜dms/opa-appa.html) with an upper intake limit of 30 mg/day. This corresponds to approximately 17 kg of seeds of the homozygous GA 17.8 line displaying the highest p-ABA level (12.8±2.7 nmol/g⁻¹ equal to 178 μg/100 gFW). Moreover, some vegetables like Brussels sprouts naturally contain relatively high p-ABA levels (8.36 nmol/g⁻¹) (Zhang et al., 2005, Rapid Commun Mass Sp 19, 963-969), supporting the fact that the pABA levels in the biofortified rice do not pose a health problem.

Similarly to pABA, levels of pterin intermediates were also elevated in GA-lines. However, they were about 80-fold lower than in the folate engineered tomato produced by Hamson's group (expressing the mammalian GTPCHI) and in the same range as in wild type tomato fruits, thus, rendering the biofortified rice perfectly safe, with regard to concerns of pterin over-consumption. Consequences of increased pteridine consumption for human are largely unknown. Humans and animals synthesize an important pterin, tetrahydrobiopterin (H₄B), which plays a role as a co-factor of NO synthases and aromatic amino acid hydroxylases and, thus, participates in the synthesis of NO and neurotransmitters, such as dopamine (reviewed in Murr, et al. Curr. Drug Metabol. 3, 175-187, 2002). Besides, the common folate and H₄B biosynthesis intermediate dihydroneopterin and its oxidized form, neopterin, are well-known markers of the immune system stimulation and are widely used in diagnostics of a numerous diseases. They possibly also participate in stress response (Oettl and Reibnegger. Curr. Drug Metabol. 3, 203-209 2002). Therefore, a misbalance of pteridine status may have serious consequences for human health.

The fact that folate biofortified rice seeds contain much less of both pABA and pteridines as compared to the engineered tomato tempts us to speculate that the usage of plant GTPCHI, which possibly retains its intrinsic negative feedback regulation, in combination with plant ADCS results in a balanced tuning of both enzyme activities, allowing optimal flux of pteridine precursors and p-ABA through the pathway towards folates. This is obviously not the case in the biofortified tomato produced by Hanson's group, where the combined overexpression of mammalian feedback-free GTPCHI and plant ADCS resulted in very high p-ABA and pteridine accumulation. Indeed, the molar ratios of folates/pABA/pterins in said folate enhanced tomatoes roughly equals to 1/2.5/0.75, while being 1/0.5/0.013 in biofortified rice of the present invention.

The maximum level of folate biofortification reached in rice seeds, namely 38.3 nmol/g, is higher than that in the biofortified tomato produced by Hanson (25 nmol/g) and corresponds to 1723 μg/100 g fresh weight, which is the highest folate content reported for plant species thus far. It exceeds the Recommended Dietary Allowance (RDA) for an adult person (400 μg) more than four-fold and is also 2.5 times above the value for pregnant women (600 μg) (National Institutes of Health. Office of Dietary Supplements. Dietary Supplement Fact Sheet: Folate. http://ods.od.nih.qov/factsheets/folate.asp). Taking into account 26±6% losses during cooking (Han et al., 2004, Eur J Clin Nutr 59:246-254) and assuming an average bioavailability of natural folates in a mixed diet of about 50% (Sauberlich et al., 1987, Am. J. Clin. Nutr. 46:1016-1028), it is very likely that 100 g of the biofortified rice grains can satisfy the daily folate requirement for an average adult person. Moreover, the fact that the biofortified rice contains almost 90% of 5-methyltetrahydrofolate makes it superior to folic acid fortified rice, mandatory in some countries. High folic acid consumption resulting from fortification may mask symptoms of vitamin B12 deficiency (Oakley, 2002, Teratology 66:44-54). This is, however, not likely to be the case when 5-methyltetrahydrofolate is used as a supplement (Gutstein et al., 1973, Dig. Dis. 18:142-146). In addition, 5-methyltetrahydrofolate has a higher efficiency in increasing red blood cell folate concentration in women of childbearing age as compared to folic acid (Lamers et al., 2006, Am. J. Clin. Nutr. 84:156-161). Therefore, biofortified rice can be a perfect alternative for regions where industrial folic acid fortification cannot be achieved for one or another reason.

In conclusion, high folate Nipponbare rice lines, capable to satisfy the RDA for an adult person with only 100 g of white rice, were generated. The biofortified rice produced in this study has a number of major advantages over the recently reported folate biofortified tomato fruits. First, the maximum absolute and relative levels of folate enhancement achieved in biofortified rice are higher than in tomato; second, engineered rice accumulates much lower levels of folate biosynthesis intermediates, p-ABA and pteridines with unknown effects on human health; third, both introduced genes reside on a single T-DNA, thus, are easily transferred to local, culinary appreciated elite varieties by conventional breeding; fourth, being one of the most important staple crops in the world, folate biofortified rice targets huge population groups, mainly in developing countries where other methods of fortification are not available or not accessible.

Rice is a major staple crop providing 80% of the daily caloric intake to 3 billion people. It is a poor source of micronutrients and vitamins, including folates (vitamin B9). Upon milling, the husk and aleurone which contain micronutrients are removed. Hence, folate shortage is wide-spread, especially in developing countries. Folate deficiency results in serious disorders, including neural tube defects as spina bifida in infants and megaloblastic anemia. Adequate dietary folate intake can prevent onset of these conditions. Folate biofortification through metabolic engineering can complement the current methods to fight folate deficiency, all of which have proven limited success. The present application reports on metabolic engineering of folate biosynthesis in rice seeds, by overexpression of two Arabidopsis genes involved in the pteridine and para-aminobenzoate branches of the pathway on a single locus. A maximal enhancement of folate content as high as 100 times above wild type levels was achieved, with 100 g of polished raw grains containing up to 4 times the adult daily requirement. However, the strategy used in this invention can also be applied to folate biofortification of plants, in particular, of other economically important graminaecious crops.

Furthermore, the transgenic plants of the present invention had a phenotype indistinguishable from the wild type throughout development and had similar seed-set capabilities. The high folate lines do not show any obvious alteration in color or smell.

### Example 6

### Feeding Tests

Rats or mice are divided in 2 groups, which are fed ad libitum by a folate repletion diet (8 mg/kg folate, control group) and folate depletion diet (0 mg/kg, experimental folate-deficient group), respectively, for 4 weeks. Then, 6 rats of each dietary group are killed; their blood is collected and folate levels are determined. The experimental folate-depleted group is divided equally into 4 subgroups, which are fed ad libitum for 4 weeks by 4 folate re-supplementation diets: 1) de-husked polished cooked wild type Nipponbare rice seeds supplemented with 8 mg/kg of folic acid; 2) de-husked polished cooked wild type Nipponbare rice seeds (˜0.18 mg folate per 1 kg raw seeds); 3) de-husked polished cooked rice seeds of the biofortified transgenic line with average folate level (˜8 mg folate per 1 kg raw seeds); 4) de-husked polished cooked rice seeds of the biofortified transgenic line with maximum folate level (˜17 mg folate per 1 kg raw seeds). After 4 weeks, 6 animals of each group are killed, blood is collected and folate levels are determined. Protocols followed may be found in for instance Murray-Kolb et al., 2002 (J. Nutrition 132: 957-960) and Miller et al. 1994 (Biochemical J. 298: 415-419.) However, alternative protocols or strategies may be used.

### Example 7

### Generation of High Folate Elite Rice Lines

Approaches which may be followed towards the generation of elite rice varieties with enhanced folate accumulation are: introgression of our high folate lines into high quality rice varieties, and transformation of our current constructs and newly generated constructs into these high quality rice lines. Given that the transformation vector allows incorporation of multiple expression cassettes, single insert, single copy events can be transferred, harbouring several genes of interest. A high folate rice transgenic line can be used as a donor parent to introgress the trait into local elite rice varieties grown in target areas with high prevalence of folate deficiency (for instance, the Guangxi province in Southwest China) by molecular marker-assisted breeding and parallel follow-up of folate levels.

Moreover, the availability of high iron and high zinc rice lines offers the opportunity to combine these traits with folate accumulation, potentially providing consumers additional protection against anemia and immune deficiency, respectively.

High folate can be combined with high iron by pyramiding the line with high folate developed by introgression and a line with high iron in the same genetic background. For instance, introgression of the folate transgenic loci can be done into the mega-variety IR64. IR64 is currently the #1 variety in India and Indonesia and is a very important variety in the Philippines and Vietnam and elsewhere in Asia, but other varieties may be used as well. Polymorphic SSR markers can be identified and used to accelerate the identification of the recurrent parental materials. In addition to the crosses mentioned above, crosses can also be made with leading high zinc breeding lines.

As an alternative approach to introduce the high folate trait in local varieties, transformation can be used.

### Example 8

### Gene Combinations Leading to Folate Enhancement

Vectors for plant transformation based on pMODULE vector type, but modified to pMOD35h (with Hygromycin resistance marker gene under CaMV35S control), containing a combination of two or three genes in folate biosynthesis, able to confer enhanced folate levels in plant seed and transgenic rice lines that possess an insertion of part of this vector conferring enhanced folate levels in rice seeds were made. Possible elements are the combined expression of folate biosynthesis genes: GTP cyclohydrolase I GTPCHI (At3g07270, full length CDS) from Arabidopsis, ADC synthase ADCS (At3g28880, full length CDS) from Arabidopsis, Dihydroneopterin aldolase 2 DHNA2 (At5g62980, full length CDS) (SEQ ID NO:45, 46) from Arabidopsis, placed under the respective control of seed specific promoters: pGlob promoter and 5′ UTR of the seed storage 26 kDa alpha globulin from Oryza sativa (japonica), corresponding to the 1130-2335 by of accession AY427569 pGluB1 promoter and 5′ UTR region of the glutelin B1 seed storage protein from Oryza sativa (japonica), corresponding to the 1130-2335 by of accession AY427569 BSV Banana streak virus promoter (partial CDS ORFIII, accession AF215815). Combinations such as GTPCHI and ADCS or GTPCHI, ADCS, and DHNA2 can confer enhanced folate levels in seed. The GluB1-ADCS-Tnos expression cassette can be inserted in the vector in either orientation. Transgenic rice lines transformed with either one of the above-mentioned gene combinations, may contain single of multiple T-DNA insertions.

### Example 9

### Plant Transformation Vectors Carrying Triple and Quadruple Folate Gene Combinations

Constructs with Folate Binding Protein (FBP, accession BT007158, CDS from 64 until 729 bp) (SEQ ID NO:39, 40); ADC lyase (ADCL, accession At5g57850 full length CDS); HPPK-DHPS (accession AJ866732, full length CDS) (SEQ ID NO:41, 42) and dihydrofolate synthase (DHFS, accession At5g41480, full length CDS) (SEQ ID NO:43, 44) have also been developed.

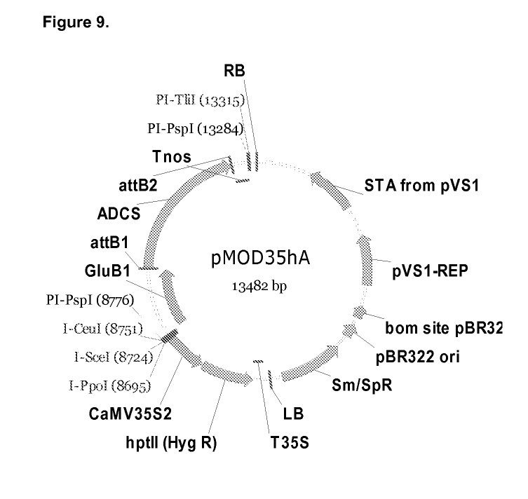

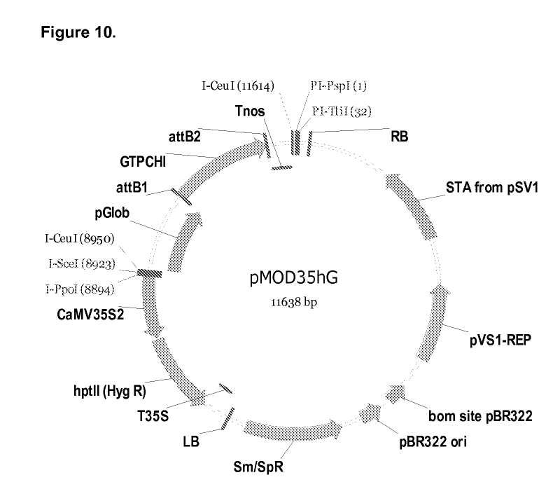

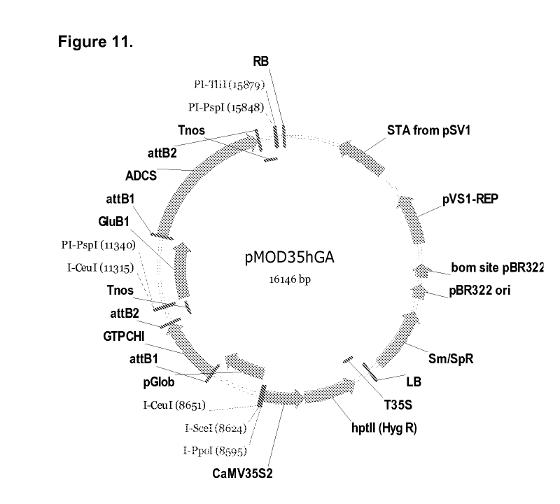

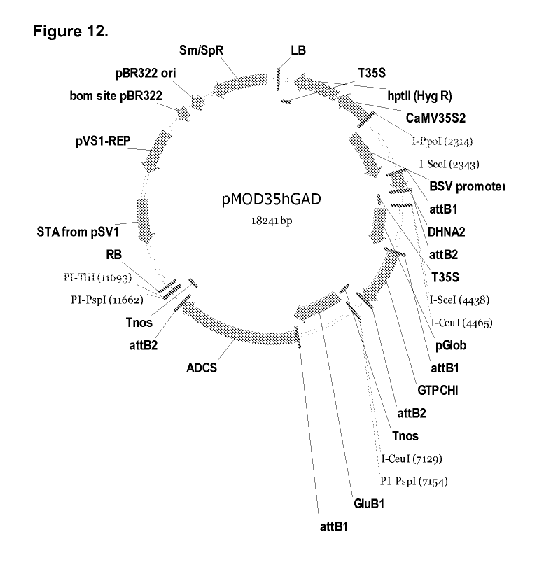

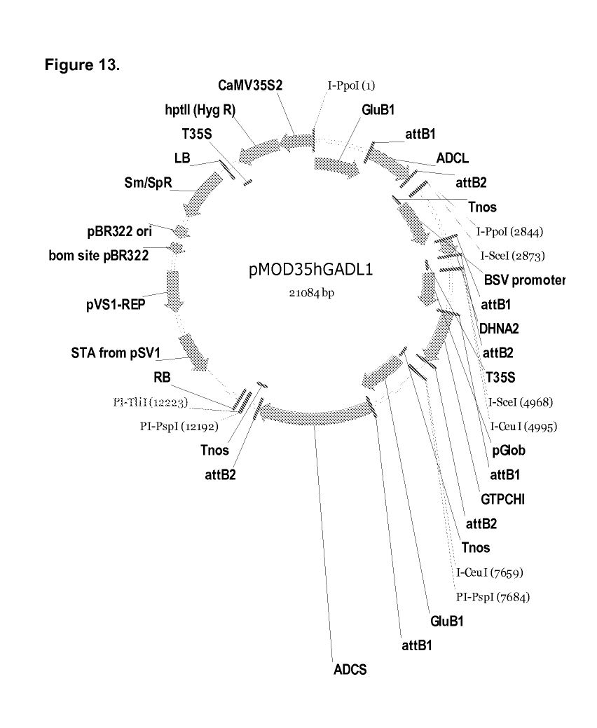

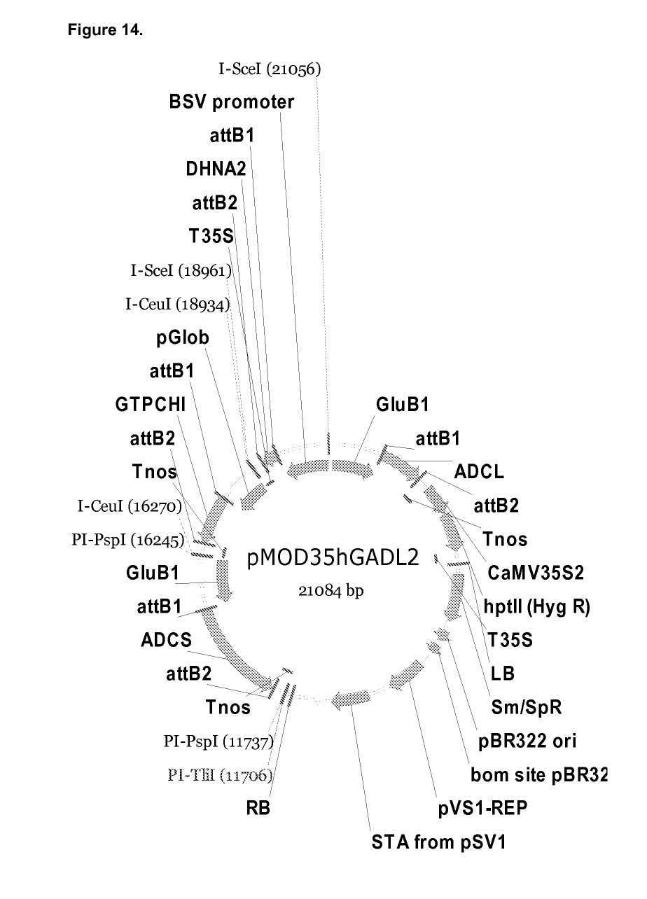

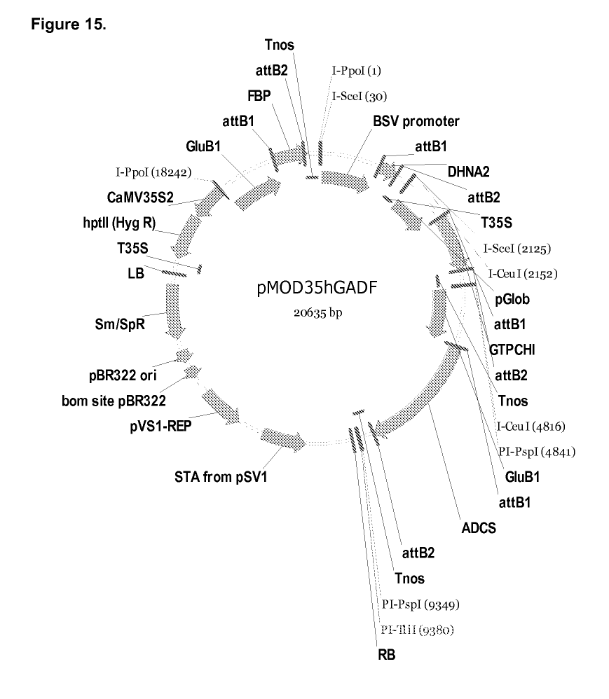

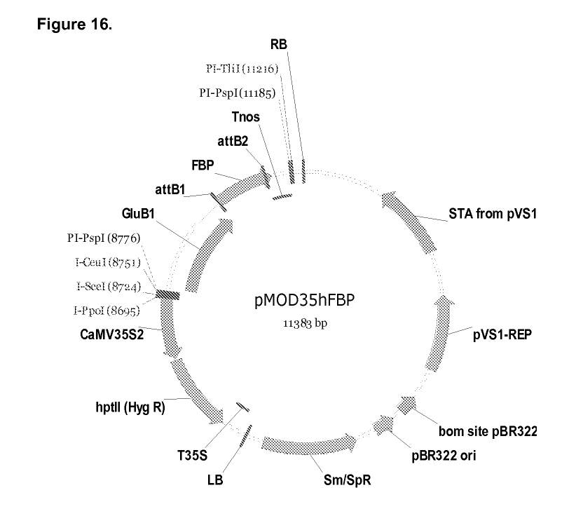

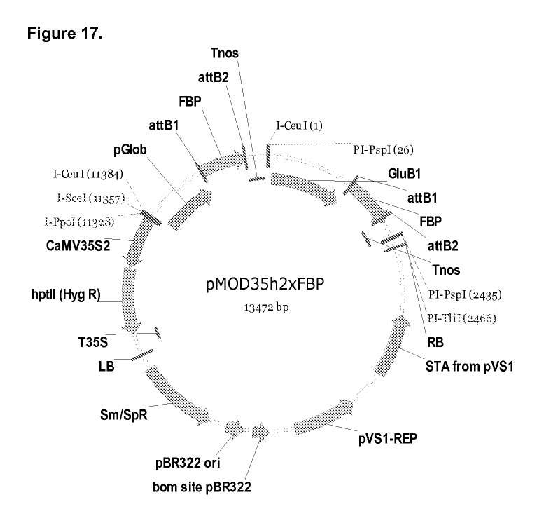

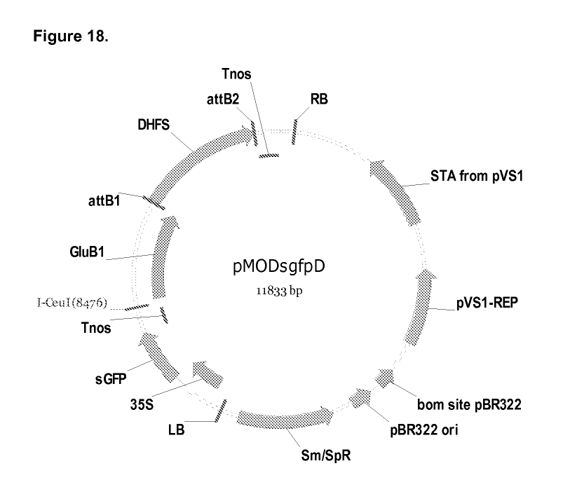

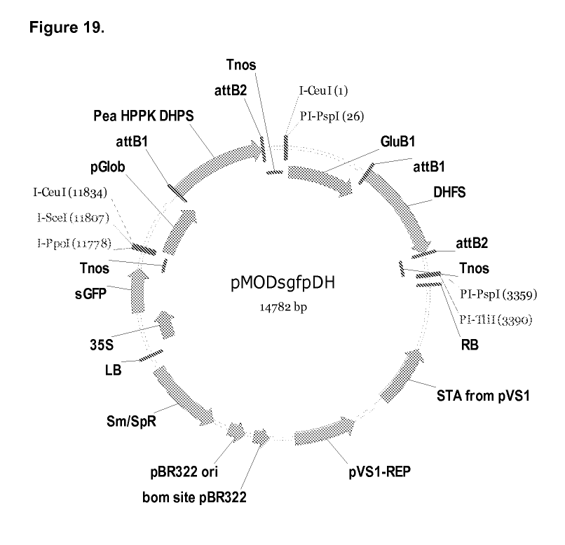

A plasmid map of the constructed vectors is shown in FIGS. 9 through 19.

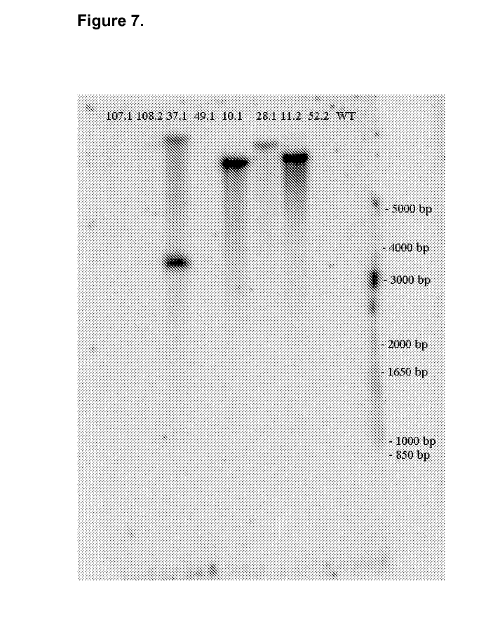

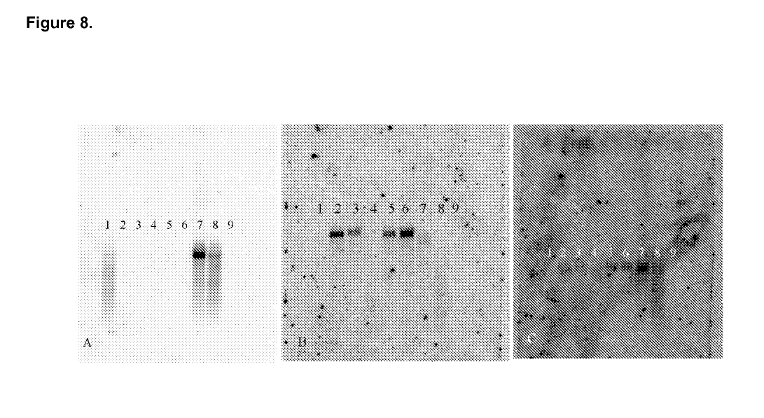

Transgenic rice plants were generated with GADL constructs and the construct integration in the genomic DNA was shown by Southern blotting-hybridization (FIG. 7). Expression of the introduced genes was studied with northern blotting (FIG. 8).

### Example 10

### Triple and Quadruple Folate Gene Combinations Lead to Similar Folate Enhancement Effects

Transgenic rice lines with single and multiple insertions containing the triple and quadruple gene combinations have been confirmed to contain folate levels approximately 30-fold higher than controls (Table 5).

### Example 11

### Folate Increase in Dicotyledonous Plants

Potato is the most important staple food after rice, wheat and maize. Potato is a dicotyledonous crop and has a low folate content (16 μg/100 gFW, American Institute of Cancer Research, http://www.aicr.org/). Using standard molecular cloning techniques, the glutelin B1 and globulin promoters in pMOD35hGA are exchanged for the potato tuber specific promoters, Pat1 and Pat2 (Rocha-Sosa et al, 1989, EMBO J., 8:23-29; Liu et al, 1991, Plant Mol Biol, 17:1139-1154). The selectable marker hptII (hygromycin resistance) is swapped for the nptII gene which confers kanamycin resistance. Transformation of potato cultivar Desiree is achieved as described (Tavazza et al, 1988, Plant Science, 59:175-181). Transformants are selected on Kanamycin medium and single insertions are identified by real time PCR as described above for rice. The procedures for folate, pABA and pterin determinations described for rice can be applied to potato. For the analysis of potato the same chromatographic and mass spectrometric conditions as used for the rice samples are applied. As to the sample preparation minor modifications are applied.

### Example 12

### GTPCHI Sequence Comparison

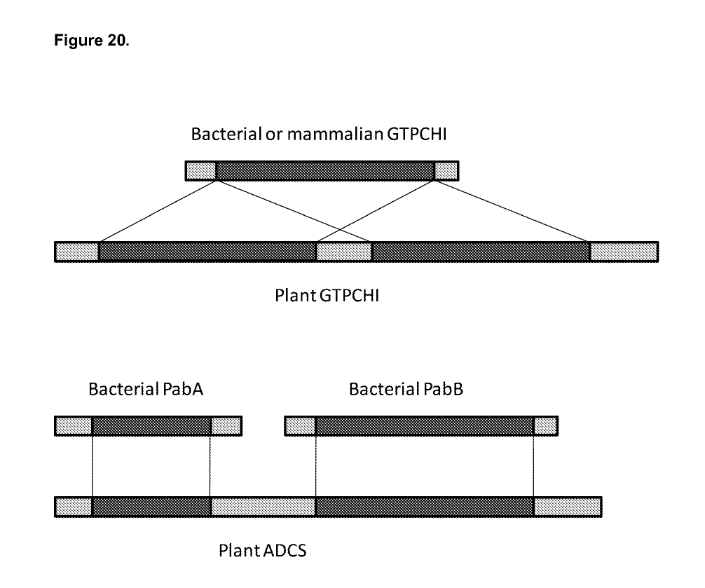

In Table 6 the percentages of identical nucleotides (amino acids) of GTPCHI cDNAs and corresponding amino acid sequences are shown. As illustrated in FIG. 20, plant GTPCHI contains two domains, with each domain being comparable to the bacterial or mammalian GTPCHI. The plant enzyme can be viewed as the duplicated bacterial or mammalian enzyme, having a duplicated domain. However these comparable domains contain only low percentages of identity (see Table 6). Furthermore, as indicated before, the regulation of said GTPCHI genes are different; having a feedback control in plant, while being non-feedback controlled in mammals (when overexpressed in plants) and bacteria. Consequently, although the mammalian and bacteria have comparable GTPCHI genes to the GTPCHI of plant, both may be considered as belonging to two distinct groups of GTPCHI genes.

The present invention relates to the use of the plant GTPCHI enzyme comprising the amino acid sequence chosen from the group consisting of a sequence represented by SEQ ID NO:2, 4, 6, 8, 10, 68 and 70, an analogue, an homologue and an enzymatically functional fragment thereof and not to the use of an GTPCHI enzyme chosen from the group consisting of a sequence represented by SEQ ID NO:60 or 62 which represent the bacterial and the mammalian GTPCHI, respectively. The present invention also relates to the use of the polynucleotide sequence encoding a plant GTPCHI enzyme chosen from the group consisting of a sequence represented by SEQ ID NO:1, 3, 5, 7, 9, 67, 69 and 71, an analogue and an homologue thereof and not to the use of a polynucleotide sequence encoding the bacterial and the mammalian GTPCHI enzyme chosen from the group consisting of a sequence represented by SEQ ID NO:59 or 61, respectively. The nucleotide and the amino acid sequences represented by SEQ ID NO: 67 an 68, respectively, relate to GTPCHI from Arabidopsis comprising a different 5′ end compared to the sequences disclosed in SEQ ID NO:1 and 2, respectively.

The present application further indicates that the analogues or homologues of the plant amino acid sequences used in the method of the present invention may be also be defined as being an analogue or a homologue of the amino acid sequences chosen from the group consisting of a sequence represented by SEQ ID NO:2, 4, 6, 8, 10, 68 and 70, wherein said analogue or homologue is at least 20.5%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% identical to one of said sequences. Said definition does not cover the corresponding bacterial or mammalian enzymes. Indeed, when considering the two domains (see Table 6), and knowing that the % of identity is lower than 41% per domain, the % identity for the whole GTPCHI between the whole plant GTPCHI enzyme and the mammalian/bacterial GTPCHI is lower than at least 20.5% identity. For the polynucleotide GTPCHI sequences no similarity could be found even when playing around with the parameters. Theoretically, for a skilled person in the art no similarity corresponds to lower than 25%, 24%, 23%, 22%, 21% or 20%.

It is to be understood that while the invention has been described in conjunction with the detailed description thereof, the foregoing description is intended to illustrate and not limit the scope of the invention, which is defined by the scope of the appended claims. All of the references cited in the description are incorporated by reference. Other aspects, advantages, and modifications are within the scope of the following claims.

1. A transgenic plant, plant organ, plant tissue, plant cell, harvestable part, propagule, seed, progeny or plant breeding material with a folate content higher than the folate content present in the wild type plant, plant organ, plant tissue, plant cell, harvestable part, propagule, seed, progeny or plant breeding material and overexpressing a plant GTP cyclohydrolase I (GTPCHI) enzyme; wherein said plant GTPCHI enzyme comprises the amino acid sequence selected from the group consisting of SEQ ID NO: 2, 4, 6, 8, 10, 68 and 70, an analogue, a homologue, and, an enzymatically functional fragment of any of SEQ ID NO: 2, 4, 6, 8, 10, 68 or 70 wherein said analogue or homologue is at least 50% identical to the amino acid sequence of any of SEQ ID NO: 2, 4, 6, 8, 10, 68 or 70.

2. The transgenic plant, plant organ, plant tissue, plant cell, harvestable part, propagule, seed, progeny or plant breeding material according to claim 1, further overexpressing a plant 4-amino-4-deoxychorismate synthase (ADCS), wherein said ADCS enzyme comprises the amino acid sequence selected from the group consisting of SEQ ID NO:12, 14 and 16, an analogue, a homologue, and, an enzymatically functional fragment of any of SEQ ID NO: 12, 14 or 16, wherein said analogue or homologue is at least 50% identical to the amino acid sequence of any of SEQ ID NO:12, 14 or 16; wherein said enzymatically functional fragment is selected from the group consisting of the putative chloroplastic transit peptide domain (1-87 aa of the polypeptide SEQ ID NO:12 or corresponding domains in SEQ ID NO:14 or 16), the putative Pab A domain (88-334 aa of the polypeptide SEQ ID NO:12 or corresponding domains in SEQ ID NO:14 or 16), and the putative Pab B domain (430-919 aa of the polypeptide SEQ ID NO:12 or corresponding domains in SEQ ID NO:14 or 16).

3. (canceled)

4. A method for increasing folate levels in a plant comprising the overexpression of a plant GTPCHI enzyme comprising the amino acid sequence selected from the group consisting of SEQ ID NO: 2, 4, 6, 8, 10, 68 and 70, an analogue, a homologue, and, an enzymatically functional fragment of any of SEQ ID NO:2, 4, 6, 8, 10, 68 or 70, wherein said analogue or homologue is at least 50% identical to the amino acid sequence of any of SEQ ID NO:2, 4, 6, 8, 10, 68 or 70.

5. The method according to claim 4, further comprising the overexpression of an ADCS enzyme comprising the amino acid sequence selected from the group consisting of SEQ ID NO:12, 14 and 16, an analogue, a homologue, and, an enzymatically functional fragment of any of SEQ ID NO:12, 14 or 16, wherein said analogue or homologue is at least 50% identical to the amino acid sequence of any of SEQ ID NO:12, 14 or 16; wherein said enzymatically functional fragment is selected from the group consisting of the putative chloroplastic transit peptide domain (1-87 aa of the polypeptide SEQ ID NO:12 or corresponding domains in SEQ ID NO:14 or 16), the putative Pab A domain (88-334 aa of the polypeptide SEQ ID NO: 12 or corresponding domains in SEQ ID NO:14 or 16), and the putative Pab B domain (430-919 aa of the polypeptide SEQ ID NO:12 or corresponding domains in SEQ ID NO:14 or 16).

6. An isolated polynucleotide comprising a polynucleotide sequence encoding a plant GTP cyclohydrolase I (GTPCHI) enzyme comprising the amino acid sequence selected from the group consisting of SEQ ID NO:2, 4, 6, 8, 10, 68 and 70, an analogue, a homologue, and, an enzymatically functional fragment of any of SEQ ID NO:2, 4, 6, 8, 10, 68 or 70, wherein said analogue or homologue is at least 50% identical to the amino acid sequence of any of SEQ ID NO:2, 4, 6, 8, 10, 68 or 70; and a polynucleotide sequence encoding a plant 4-amino-4-deoxychorismate synthase (ADCS) enzyme comprising the amino acid sequence selected from the group consisting of a sequence SEQ ID NO:12, 14 and 16, an analogue, a homologue, and, an enzymatically functional fragment of any of SEQ ID NO:12, 14 or 16, wherein said analogue or homologue is at least 50% identical to the amino acid sequence of any of SEQ ID NO:12, 14 or 16; and wherein said enzymatically functional fragment is selected from the group consisting of the putative chloroplastic transit peptide domain (1-87 aa of the polypeptide SEQ ID NO:12 or corresponding domains in SEQ ID NO:14 or 16), the putative Pab A domain (88-334 aa of the polypeptide SEQ ID NO:12 or corresponding domains in SEQ ID NO:14 or 16), and the putative Pab B domain (430-919 aa of the polypeptide SEQ ID NO:12 or corresponding domains in SEQ ID NO:14 or 16).

7. A method of increasing the folate content of a plant comprising transforming plant cells with the isolated polynucleotide according to claim 6, and regenerating a transgenic plant from the transformed plant cells.

8. An expression vector comprising the polynucleotide according to claim 6 and regulatory sequences operatively linked to said polynucleotides such that the polynucleotides are expressed in the plant cell in order to increase the level of folate in said plant.

9. A transgenic plant cell transformed with the expression vector according to claim 8.

10. A transgenic plant, plant organ, plant tissue, harvestable part, propagule seed, or progeny with an increased folate content derived from the transgenic cell according to claim 9.

11. A product, a food additive or a pharmacological composition comprising a transgenic plant, plant organ, plant tissue, plant cell, harvestable part, propagule, seed, or progeny according to claims 1, 2 or 10.

12. A medicament comprising a transgenic plant, plant organ, plant tissue, plant cell, harvestable part, propagule, seed, or progeny according to claim 1, 2, or 10.

13. A method of biofortification to enrich the quality of the food of an animal or human subject comprising propagating the transgenic plant, plant organ, plant tissue, harvestable part, propagule seed, or progeny according to claim 1, 2, or 10.

14. A method of reducing the risk of or treating folate insufficiency or diseases or disorders resulting from said insufficiency in an animal or human subject by administering a product, a food additive or a pharmacological composition according to claim 11 to an animal or human subject in need thereof.

15. A medicament comprising a product, additive or composition according to claim 11.
